(19) United States
(12) Patent Application Publication
(10) Pub. No.: US 2009/0149871 A9
(43) Pub. Date: Jun. 11, 2009

(54) Devices and methods for treating morbid obesity

(76) Inventors: Jonathan Kagan, Hopkins, MN (US); Mitchell Dann, Wilson, WY (US); Greg Fluet, Jackson, WY (US); Sayeed Ikramuddin, Minneapolis, MN (US); Paul Swain, London (GB); Richard Thomas, Belmont, MA (US); Gerard von Hoffmann, Trabuco Canyon, CA (US); Mary Lynn Wilmore, Victor, ID (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/025,364

(22) Filed: Dec. 29, 2004

Related U.S. Application Data
(63) Continuation-in-part of application No. 10/698,148, filed on Oct. 31, 2003.
(60) Provisional application No. 60/534,056, filed on Dec. 31, 2003.
(60) Provisional application No. 60/569,442, filed on May 7, 2004.
(60) Provisional application No. 60/613,917, filed on Sep. 27, 2004.
(60) Provisional application No. 60/422,987, filed on Nov. 1, 2002.
(60) Provisional application No. 60/428,483, filed on Nov. 22, 2002.
(60) Provisional application No. 60/430,857, filed on Dec. 3, 2002.
(60) Provisional application No. 60/437,513, filed on Dec. 30, 2002.
(60) Provisional application No. 60/448,817, filed on Feb. 21, 2003.
(60) Provisional application No. 60/480,485, filed on Jun. 21, 2003.

(51) Int. Cl. A61B 17/08

(52) U.S. Cl. 606/151; 606/157

(57) ABSTRACT

The present invention provides devices and methods for attachment of an implanted device, such as an artificial stoma device, a gastrointestinal sleeve device or an attachment cuff, within a patient's digestive tract for treatment of obesity. Special surgical fasteners provide a lasting and durable attachment to the gastrointestinal tissue without causing excessive pressure that could result in tissue erosion and detachment of the implanted device. Fastener delivery devices that facilitate peroral placement and deployment of fasteners and secondary devices are also provided. Also described are implantable devices and attachment means that avoid causing excessive pressure within the tissue by having compliance that is compatible with the gastrointestinal tissues where it is attached.

### CROSS REFERENCE TO RELATED APPLICATIONS

This patent application claims the benefit of U.S. provisional patent application 60/534,056, filed on Dec. 31, 2003, by Kagan et al. for Devices and Methods for Treating Morbid Obesity, U.S. provisional patent application 60/569,442, filed on May 7, 2004, by Kagan et al. for Devices and Methods for Treating Morbid Obesity and U.S. provisional patent application 60/613,917, filed on Sep. 27, 2004, by Kagan et al. for Devices and Methods for Attachment of a Gastrointestinal Sleeve. This patent application is also a continuation-in-part of U.S. utility patent application 10/698,148, filed on Oct. 31, 2003 by Kagan et al. for Apparatus and Methods for Treatment of Morbid Obesity. The devices and methods described herein can be combined with and/or used in conjunction with the apparatus and methods described in these prior applications. These and all patents and patent applications referred to herein are hereby incorporated by reference in their entirety.

### FIELD OF THE INVENTION

The present invention relates generally to apparatus and methods for treatment of obesity, and particularly morbid obesity. In particular, it relates to apparatus and methods that can be applied using minimally invasive techniques for effectively reducing stomach volume, bypassing a portion of the stomach and/or small intestines and/or reducing nutrient absorption in the stomach and/or small intestines. The present invention also relates to devices and methods for attachment of a gastrointestinal sleeve device within a patient's digestive tract for treatment of obesity.

### BACKGROUND OF THE INVENTION

Gastrointestinal sleeve devices for treatment of obesity have been described in the prior applications listed above, as have various devices and methods for attachment of a gastrointestinal sleeve device within a patient's digestive tract. The present invention is the result of continued investigation into devices and methods for attachment of a gastrointestinal sleeve device within a patient's digestive tract.

### SUMMARY OF THE INVENTION

One objective of the present invention is to achieve a lasting, durable attachment for devices implanted within the gastrointestinal tract, and preferably an attachment that is reversible for later removal and also preferably enables placement, removal and/or replacement of another implanted device. To achieve this it is important to avoid tissue erosion at the attachment points, particularly as a result of pressure necrosis. Excessive pressure on the gastric or esophageal wall frequently leads to pressure necrosis, which can result in detachment of the implanted device. Excessive pressure can occur at attachment points or at any interface between the tissues and an implanted device. Motion of the gastric or esophageal wall due to expansion and contraction due to stomach contents or muscular peristaltic action can create or exacerbate excessive pressure in the tissues, which may lead to ischemia, pressure necrosis and tissue erosion. Avoiding pressure is key to preventing ischemia, pressure necrosis and tissue erosion at the attachment points and any other interface between the tissues and the implanted device.

One aspect of the present invention is to provide surgical fasteners that afford a secure attachment to the gastrointestinal tissue without causing excessive pressure within the tissue. The surgical fasteners can be used for attachment of an artificial stoma device, a gastrointestinal sleeve device or an attachment cuff, to which another implantable device can be attached. Fastener delivery devices that facilitate deployment and peroral placement of secondary devices, e.g. an attachment cuff, are also provided. Fasteners are provided that are especially adapted for convenient and reliable deployment using endoscopic methods via a peroral approach. Fastener delivery devices that facilitate deployment of the fasteners are also provided.

Another aspect of the present invention is to provide an implantable device and/or attachment means that avoids causing excessive pressure within the tissue by having compliance that is compatible with the gastrointestinal tissues where it is attached. Device compliance can also be important for providing a leak free seal between an implanted device and the tissue at the attachment point. Compliance can be provided in the radial or circumferential direction and/or in the vertical, axial or longitudinal direction. The device may have different compliance in different regions to be compatible with the tissue at the attachment point and at other portions of the gastrointestinal tract through which it runs. The device may have different compliance in different directions to be compatible with the tissue at the attachment point while simultaneously achieving other goals of the device. Compliance can be provided in a number of different ways. One way is by elastic or plastic deformation of the device and/or the attachment means. Another way is by a mechanical decoupling that allows relative movement between the device and the attachment points, and/or between the attachment points themselves, without transmitting excessive force or pressure to the tissue.

In some clinical situations, it will be desirable to match compliance between the device and the tissue to which it is attached. In other situations, based upon the clinical situations, it will be desirable to provide a device with higher or lower compliance than the tissue to achieve certain objectives. For example, maintaining the position of the proximal end of an attached sleeve device will require a device that is relatively noncompliant in at least the axial direction.

Compliance of GI tissue can be a time and/or rate dependant phenomenon. GI tissue will resist abrupt tensile forces but will relax and stretch out if force is applied over a long period of time. Compliance of GI tissue also changes with stimulation of the muscular layers. This can be neural and/or hormonal. GI tissue also has a tendency to return to a resting configuration and/or shrink or contract if not exposed to mechanical stress. The compliance of GI tissue can also change. For example, fibrosis and/or scaring effects that may occur at a plication or attachment point would be expected to reduce tissue compliance.

The tissue at the GEJ or cardia can move upward toward the esophagus (e.g. when swallowing or retching) and downward and outward toward the fundus (e.g. when the stomach is engorged). This will then result in a change in the diameter of the tissue ring at the GEJ/cardia. This will be associated with a proportional change in the circumference of the cuff/ring, which will require the cuff material to fold/collapse (with upward motion) or extend/stretch (with downward motion).

The device or fastener may be configured to have a compliance limit. The material stretches to a point (limit) and then will stretch no more. When GI tissue stretches naturally it is only subjected to the force required to stretch the tissue. For GI tissue attached to a cuff or sleeve, when attached to a relatively less compliant material it can be subjected to the additional forces required to stretch the less compliant material. When attached to a material with relatively greater compliance there should be little additional force on the tissue. This would be true until a compliance limit of the material was reached after which appreciable additional forces could be seen by the tissue.

In the case of isolated attachment points (i.e. individual unconnected hangers) the same principles apply, however, in this case it may not be the cuff that connects the attachment points. In this case it can be the sleeve (when in place) that connects and transmits forces between the attachment points. In this case the compliance of the sleeve at the attachment point defines the compliance limit.

An intermediate or transitional proximal sleeve/cuff can be used to decouple a relatively noncompliant sleeve from the GI tissue without use of isolated attachment points. This can be integrated with or separate from the sleeve. A separate and removable intermediate cuff can be constructed similarly to a primary attachment cuff and similarly remain in place when sleeves are removed and/or replaced. It may be desirable to revise or replace a sleeve device to increase or decrease the therapeutic effect and/or to remove a sleeve after a suitable therapeutic interval. Apparatus are disclosed where relative motion between the fasteners and/or interconnected devices can be allowed as an alternate means to decouple a relatively noncompliant sleeve from the GI tissue.

Methods and apparatus are also provided for allowing tissue healing at attachment points and prestrengthening or thickening the tissue prior to attachment of an implantable device. In addition, sutures and surgical fasteners are provided that can accommodate tissue thickening without causing excessive pressure within the tissue.

Accordingly, the present application, the referenced provisionals and the parent application describe devices, features, means and methods that can be used alone or in combination to achieve a lasting, durable attachment for devices implanted within the gastrointestinal tract. Furthermore, this is preferably an attachment that is reversible for later removal and also preferably enables placement, removal and/or replacement of another implanted device.

### BRIEF DESCRIPTION OF THE DRAWINGS

FIG. 1A shows a gastrointestinal sleeve device attached to an artificial stoma device implanted within a patient's stomach.

FIG. 1B shows a gastrointestinal sleeve device attached at the GEJ with an attachment cuff.

FIG. 1C shows a gastrointestinal sleeve device with a tapered or funnel-shaped entry implanted within a patient's stomach.

FIGS. 2A-2B show a T-tag fastener with a spacer to avoid excessive pressure on the tissue.

FIGS. 3A-3D show another T-tag fastener with a spacer to avoid excessive pressure on the tissue.

FIG. 4 illustrates a method and apparatus for placing T-tag fasteners at the gastroesophageal junction (GEJ).

FIG. 5 shows a device being parachuted into place along the suture tails.

FIGS. 6A-6G illustrate a dual-headed T-tag fastener.

FIGS. 7-12 show the steps for deploying a dual-headed T-tag fastener.

FIGS. 13A-13D are detail drawings of a delivery device for deploying a dual-headed T-tag fastener.

FIGS. 14A-14C show variations of a rail-mounted delivery device for deploying a T-tag fastener mounted on the exterior of a flexible endoscope.

FIGS. 15A and 15B show another embodiment of a delivery device for deploying a T-tag fastener mounted on the exterior of a flexible endoscope.

FIG. 16 is a detail drawing of a pusher for use with a T-tag fastener delivery device.

FIG. 17 shows a proximal end of a delivery device with a magazine for sequentially delivering multiple T-tag fasteners.

FIGS. 18A-18E show examples of flexible constructions for a delivery cannula.

FIG. 19A shows a snap T-tag fastener.

FIG. 19B shows the snap T-tag fastener of FIG. 19A with the cap in place.

FIGS. 20A-20B show the parachute T-tag of a fastener and a snap of a T-tag fastener positioned outside the slotted delivery cannula.

FIGS. 21, 22A-22B and 23 show another configuration of a T-tag fastener delivery device.

FIGS. 24A-24D show a method of T-tag fastener delivery with the suture tail inside of the penetrating cannula.

FIGS. 25A-25D show a slotted penetrating cannula with a pusher configured to exclude the suture tail of the T-tag fastener from the slot.

FIGS. 26A-26B show a method of orienting a T member of a fastener after insertion.

FIG-27 shows another method of orienting a T member of a fastener after insertion.

FIGS. 28A-28C show embodiments of a grasping device combined with an attachment device.

FIGS. 29A-29H show different possible configurations of T members.

FIGS. 30A-30B illustrate an attachment ring for a gastrointestinal sleeve device.

FIGS. 31-32 show an attachment ring device that uses a double plication for attachment to the gastric wall.

FIGS. 33A-33B illustrate another means for attaching a gastrointestinal sleeve device.

FIGS. 34 and 35A-35C show an example of vertically mounted isolated sliding attachment members in a patient's stomach.

FIGS. 36-37 show a compliant fastener that can accommodate large gastric wall motions.

FIGS. 38A-38D show an embodiment of a flexible attachment device for implantable morbid obesity treatment devices.

FIGS. 39A-39B, 40A-40B and 41A-41B show embodiments of a flexible attachment device.

FIGS. 42A-42C show embodiments of an attachment device that are configured with a flaring or outward curve at the upper end of the cylindrical wall to minimize pressure concentrations where it contacts the stomach wall.

FIG. 43 is a cutaway drawing showing the internal construction of an embodiment of a flexible attachment device.

FIG. 44 shows an attachment device for attaching a treatment device that is larger in diameter than the attachment device.

FIG. 45 illustrates an attachment cuff with an external sleeve attachment interface.

FIG. 46 illustrates an attachment cuff with separation of the attachment and sealing functions.

FIGS. 47A-47C illustrate an embodiment of an attachment cuff with controlled compliance volume change.

FIGS. 48A-48C illustrate another embodiment of an attachment cuff with controlled compliance volume change.

FIGS. 49A-49F illustrate a method and apparatus for attaching a gastrointestinal sleeve device.

FIGS. 50A-50B illustrate a method and apparatus using an extragastric structure for attaching a gastrointestinal sleeve device.

FIGS. 51A-51E show details of an embodiment of an extragastric structure for attaching a gastrointestinal sleeve device.

FIGS. 52-53 show examples of tissue prestrengthening in the gastrointestinal system.

FIGS. 54-55 show examples of tissue thickening in the gastrointestinal system.

FIG. 56 shows an example of tissue thickening used to create a restrictive stoma at the GEJ.

FIGS. 57A-57C show the effect of tissue thickening on a fixed length suture or fastener.

FIGS. 58A-58C show controlled suture lengthening to compensate for tissue thickening.

FIGS. 59A-59E illustrate an embodiment of a fastener with controlled suture lengthening to compensate for tissue thickening.

FIGS. 60A-60B illustrate another embodiment of a fastener with controlled suture lengthening to compensate for tissue thickening.

FIGS. 61A-61B illustrate another embodiment of a fastener with controlled suture lengthening to compensate for tissue thickening.

### DETAILED DESCRIPTION OF THE PREFERRED EMBODIMENT

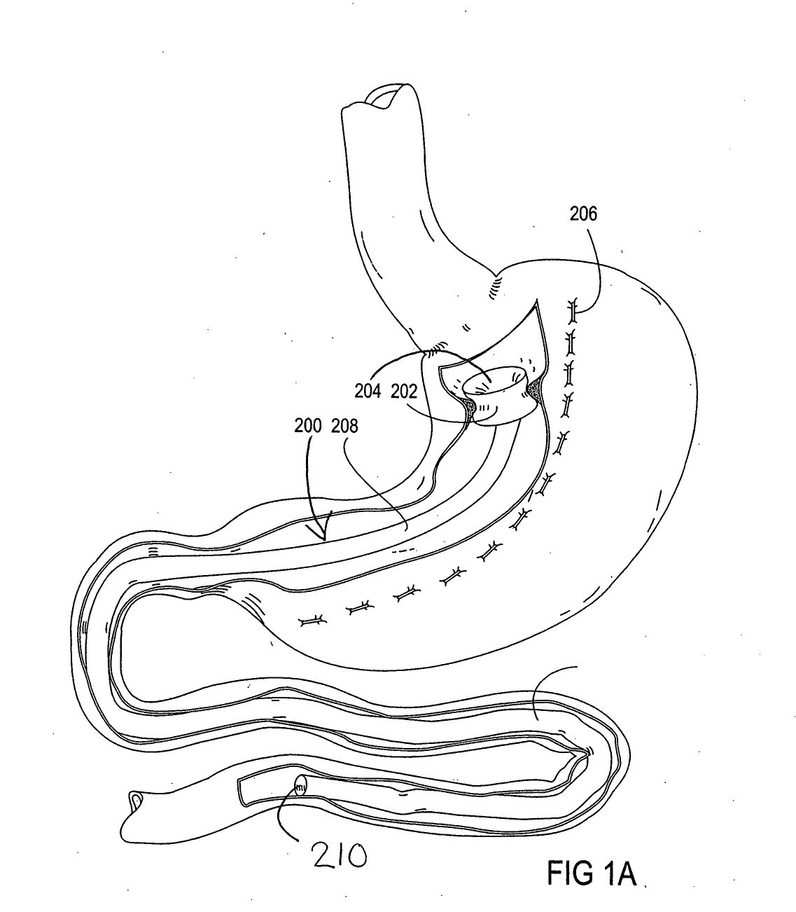

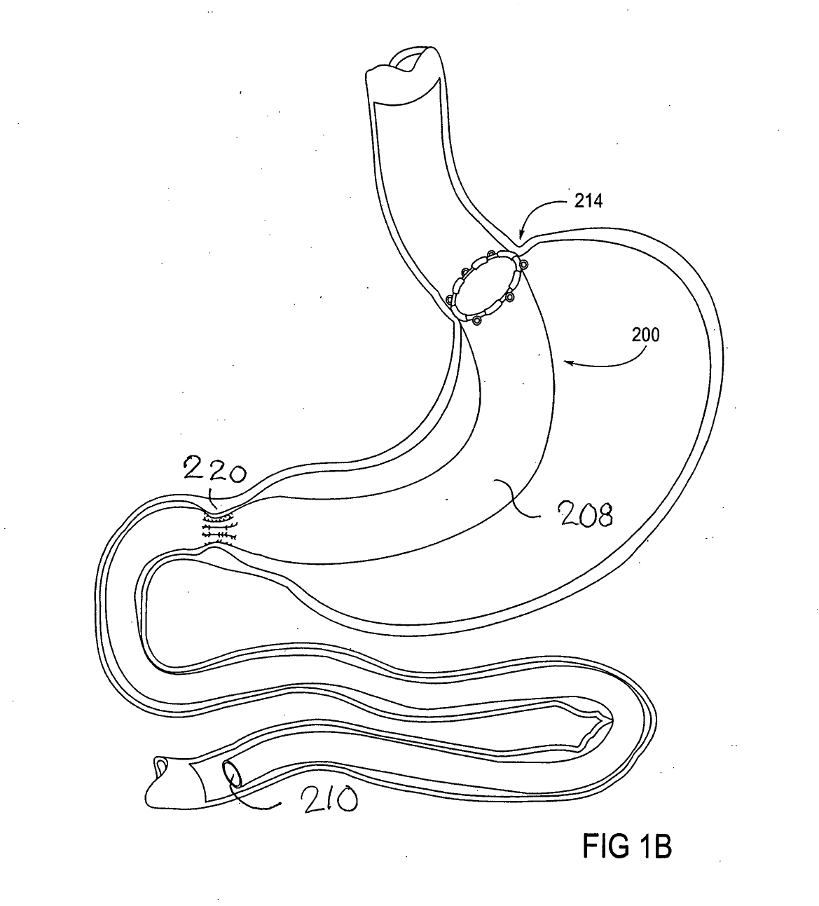

The parent application, Ser. No. 10/698,148, describes gastrointestinal sleeve devices that can mimic a Roux-en-Y gastric bypass by effectively reducing stomach volume, bypassing a portion of the stomach and/or small intestines, reducing nutrient absorption in the stomach and/or small intestines and depositing minimally or undigested food farther than normal into the intestines, thereby stimulating intestinal responses. The gastrointesintal sleeve devices described therein are all adaptable for use with the apparatus and methods of the present invention. FIGS. 1A-1C show representative examples of such gastrointestinal sleeve devices.

FIG. 1A shows a gastrointestinal sleeve device 200 attached to an artificial stoma device 202 implanted within a patient's stomach. The artificial stoma device 202 can be implanted at the outlet of a surgically created gastric pouch to create a restriction that limits the volume of food that can be ingested at one time. The artificial stoma device 202 can have a fixed diameter stoma opening 204 or it can have an adjustable stoma opening or it can be a “smart” stoma that adjusts the size of the stoma opening in response to various conditions. The artificial stoma device 202 is preferably configured for peroral delivery and attachment using endoscopic techniques. Alternatively, the artificial stoma device 202 can be implanted using laparoscopic or open surgical techniques. The gastrointestinal sleeve device 200 is an elongated flexible tubular structure that is attached to the artificial stoma device 202 such that food and liquids pass through the stoma opening 204 and enter the internal lumen 208 of the sleeve device 200. The artificial stoma device 202 and the gastrointestinal sleeve device 200 can be implanted simultaneously, or the artificial stoma device 202 can be implanted by itself and then the gastrointestinal sleeve device 200 can be attached to the artificial stoma device 202 in the same or a subsequent procedure. The stomach will tend to shrink around the sleeve device over time due to disuse, reducing the stomach volume and increasing peristaltic coupling between the stomach wall and the sleeve device. Optionally, a line of staples or other fasteners 206 may be used with any of the devices to create a gastroplasty to reduce the volume of the stomach.

In conjunction with the stoma and/or gastric sleeve, the volume of the stomach can be reduced by suturing, stapling using open, transesophageal or laparoscopic techniques. Alternatively or in addition, a gastric balloon or other volume displacement device may be used in conjunction with the gastric sleeve to provide a feeling of satiety. These adjunctive techniques have the effect of further reducing nutrient intake (in the case of a stomach reduction and pouch formation upstream of a stoma) and enhancing the effect of peristaltic motions of the stomach for moving food through the gastric sleeve intake (in the case of a stomach reduction downstream of a stoma where there is a gastric sleeve).

FIG. 1B shows a gastrointestinal sleeve device 200 attached at the GEJ with an attachment cuff 214. The attachment cuff 214 and the gastrointestinal sleeve device 200 can be implanted simultaneously, or the attachment cuff 214 can be implanted by itself and then the gastrointestinal sleeve device 200 can be attached to the attachment cuff 214 in the same or a subsequent procedure. Optionally, the attachment cuff 214 can be allowed to heal for a period of time before attaching the gastrointestinal sleeve device 200. Additionally, the gastrointestinal sleeve device 200 can be later removed or replaced without removing the attachment cuff 214. In this example, the volume of food ingested is limited by the portion of the sleeve device 200 upstream of the pylorus 220 rather than by a restrictive stoma. Furthermore, attachment at the gastroesophageal junction excludes all gastric secretions and food from the interior of the gastrointestinal sleeve device 200.

FIG. 1C shows a gastrointestinal sleeve device 200 with a tapered or funnel-shaped entry 216 implanted within a patient's stomach. In this example the funnel-shaped entry 216 creates a reduced volume gastric pouch that also includes a portion of the gastric wall, so that stretch receptors in the gastric wall will help to create a feeling of satiety. In this example, the proximal end 218 of the gastrointestinal sleeve device 200 is attached directly to the gastric wall at the cardiofundal border. Alternatively, an attachment cuff or other attachment means may be used. Optionally, the gastrointestinal sleeve device 200 may include an artificial stoma or other restriction at the base of the funnel-shaped entry 216.

In each of these examples, the gastrointestinal sleeve device 200 preferably has a length such that ingested food and liquids bypass most of the stomach and at least a portion of the small intestine. Undigested food and liquids exit the distal end 210 of the sleeve device 200 into the small intestine reducing caloric absorption and eliciting physiological responses within the intestines. The gastrointestinal sleeve device 200 can have a constant diameter throughout its length or the diameter may vary along the length. The gastrointestinal sleeve device 200 can be impermeable along the entire length or some or all of the device may be porous or semipermeable. Preferably, the wall of the gastrointestinal sleeve device 200 is thin and flexible so that peristalsis is coupled to the internal lumen 208 of the device. A gastric sleeve that extends beyond the pylorus 220, with or without an intestinal sleeve, can allow use of the pylorus as a natural stoma by configuring the sleeve to close by the pylorus and then open to allow passage of food when the muscles of the pylorus relax. The section of the sleeve device 200 that passes through the pylorus 220 will preferably have enough wall flexibility or compliance to allow normal opening and closing of the pylorus to release and retain stomach contents and to allow drainage of stomach secretions around the outside of the sleeve. This can optionally be accomplished by the inclusion of pleats, channels or other structures to facilitated the collapse and sealing of the sleeve as well as passage of gastric secretions along the outside of the sleeve as shown in FIG. 1B.

Structures, features and methods illustrated in FIGS. 1A-1C can be combined or interchanged based upon clinical requirements. Similarly, dimensions, materials and other specifications described in the 10/698,148 application can be adjusted based upon the clinical situation. For example, the gastrointestinal sleeve 200 is preferably approximately 60-180 cm in length whereby partially digested or undigested nutrients exit from the sleeve into the jejunum where they can elicit a hormonal, neural and/or osmotic reaction in the jejunum and/or ileum. Increasing the length of the sleeve can increase the degree of response in the ileum.

Optionally, the sleeve can include coatings on its interior and/or exterior to enhance the surface properties of the sleeve in clinically relevant manners. Coating examples include: 1) parylene coatings to increase the chemical resistance of a sleeve material, 2) coating with an antimicrobial agent to resist infection and/or 3) coating with an anti-inflammatory agent to reduce tissue inflammatory response, as described herein. Similarly, the interior and exterior of the sleeve can optionally be coated with a low friction material (e.g. a hydrogel) to reduce friction of food passage (interior) and reduce gastric irritation (exterior).

The 10/698,148 application describes the use of biodegradable or bioresorbable materials for construction of a gastrointestinal sleeve device to obviate the need for removal of the sleeve device at the end of the treatment period. The entire gastrointestinal sleeve device or a portion of it may be made of biodegradable material. The gastrointestinal sleeve device may be made of biodegradable materials with different rates of degradation or resorption. The gastrointestinal sleeve device may be configured with a series of segments that biodegrade sequentially. For example, a first portion on the distal end of the sleeve may degrade first, followed some time later by a second intermediate portion and a third proximal portion. Next the attachment would degrade and, finally, the T-tags or other fasteners would degrade. Alternatively, the gastrointestinal sleeve device may be configured with a series of short segments of non-biodegradable material that are attached to one another with biodegradable material. The biodegradable attachment portions may be made of biodegradable materials with different rates of degradation or resorption so that they biodegrade sequentially. In either case, the biodegradable material would allow a gradual change of therapy over time, without having to revise or replace the implant. The patient could get used to the gradual change in therapy more readily than a sudden change and would be better able to avoid a rebound in weight gain. It would also allow for a safe mode of degradation and elimination. The device would degrade into pieces small enough that they could be eliminated without any danger of bowel obstruction.

Alternatively, selected portions of the gastrointestinal sleeve device may be made of biodegradable material. For example, openings in the sleeve can be covered with biodegradable material that will gradually degrade over time, eventually allowing food to mix with digestive secretions. The biodegradable material would allow a gradual change of therapy over time, without having to revise or replace the implant. The gastrointestinal sleeve device with the openings in it could be left in place for long-term maintenance of weight loss or it could eventually be removed.

Biodegradable material suitable for construction of a gastrointestinal sleeve device is sold under the name Plastifilm by OsteoBiologics, Inc., located in San Antonio, Tex. This biodegradable polymeric film material is described in U.S. Pat. 6,514,286, which is hereby incorporated by reference. Additional information from the supplier about this material is available at: http://www.obi.com/.

Another aspect of the present invention involves devices and methods for delivery and deployment of a gastrointestinal sleeve device into a patient's gastrointestinal tract. One method to facilitate delivery of the device into and through the patient's small intestine is to place a guidewire and/or catheter into the intestine to the depth desired and then push the gastrointestinal sleeve device over the guidewire. Successful techniques for placing a guidewire into the small intestines have been described by G. Long, T. Mills and C. P. Swain in an article entitled Techniques for advancing guide wires and devices in the lumen of the gastrointestinal tract. Another technique that could be adapted for placing a device such as a gastrointestinal sleeve device into the small intestine was described by H. Yamamoto and K. Sugano in an article entitled A new method of enteroscopy—the double-balloon method, Can J Gastroenterol. 2003 April;17(4):273-4. These techniques can be used in combination with many of the delivery and deployment methods described herein and in the prior application.

The 10/698,148 application describes gastric and gastrointestinal sleeve devices that include inflatable balloons for structural support of the sleeve and/or for enhancing the patient's feeling of satiety. An enhanced method of using these devices is to inflate the balloons with a fluid containing a nontoxic detectable dye, such as methylene blue. If any of the inflatable balloon members should develop a leak, the methylene blue will be passed in the urine and be detectable by the patient. The patient should then contact a physician to determine whether repair or replacement of the device is indicated.

Another concept described in the 10/698,148 application involves the placement of a mounting ring or other attachment device within the gastrointestinal system and attaching various other devices or components to the attachment device. Enhancements to that concept for treating GERD, MO and other disorders of the gastrointestinal tract could include placing/attaching a nonrestrictive mounting ring at or near the GEJ and attaching/removing/replacing various therapeutic or diagnostic devices to the mounting ring, such as a valve to prevent reflux, a restriction to food intake, a sleeve, a telemetry or imaging capsule, etc.

Methods of insertion and retrieval of a gastrointestinal sleeve device are also described in the parent application. In addition to the methods described therein, a GI sleeve can be inserted and/or retrieved using a flexible endoscope. A skilled GI endoscopist can “drive” a special endoscope (an enteroscope) through the duodenum and deep into the jejunum. With proper interfacing structure on a GI sleeve, the sleeve can piggyback on the endoscope as it is driven into the jejunum and then released with its distal end left in the jejunum when the endoscope is retracted and removed from the body. This can be accomplished perorally either before or after attachment of the proximal end of the sleeve to the GEJ or some other clinically desirable location.

Various means can be used as an interface between the endoscope and the distal end of the GI sleeve device. If the sleeve device has a solid distal end or other graspable portion, such as a tab or loop near the distal end, a standard or custom endoscopic snare or grasper can be extended through the endoscope working channel to grasp the sleeve device. Alternatively, the distal end of the sleeve device can be configured with a socket or pocket to engage a flexible pusher, which may be configured as a rod, tube or guidewire. As another alternative, the sleeve device can be configured with a distal end that can be cut off to release the device. The distal end of the sleeve device is grasped with a snare or the like extended through the endoscope working channel. Once the sleeve device is delivered far enough distally in the GI tract, the distal end of the sleeve device is cut off to release the device.

The method of delivery is different depending upon whether or not the proximal end of the sleeve is attached in the GI tract before sleeve delivery. It is much simpler if distal delivery is performed prior to attaching the proximal end of the sleeve, therefore this method is described. The following method is exemplary and positions the sleeve coaxial (outside) the endoscope. With minor modifications the same method can be applied to a parallel (side by side) delivery.

The distal end of the GI sleeve device can be delivered through the stomach and into the small intestine by the following steps:
1) Slide endoscope through or around sleeve
   i) Flush sleeve with saline if through the sleeve
2) Slide endoscopic interface component (e.g. snare) though endoscope biopsy channel
3) Place sleeve interface in snare
4) Engage and lock snare
5) Retract snare flush with distal tip of the endoscope
6) Pass endoscope and sleeve through the esophagus
   i) Take care not to damage the sleeve on the teeth or anywhere else
7) Pass endoscope and sleeve through the stomach and pylorus
   i) Take care not to damage the sleeve on the teeth or anywhere else
8) Pass endoscope and sleeve through the duodenum and into the jejunum
   i) Take care not to damage the sleeve on the teeth or anywhere else
9) Slightly advance snare and visually check snare and distal interface for tangles, etc.

One option for release of the GI sleeve device includes the following steps:
10) Release snare
11) Advance snare past the distal sleeve interface
   i) Clear distal interface from snare, confirm visually
12) Retract snare into endoscope, optionally remove snare totally
13) Optionally secure distal sleeve interface to jejunum with a clip or other fastener
   i) Clip or fastener will be sloughed from jejunum wall by natural processes
14) Remove endoscope
   i) Take care not to dislodge or displace sleeve
15) Attach proximal sleeve to GEJ or other anatomy
   i) Optionally attach to an interface cuff, etc.

Another option for release of the GI sleeve device includes the following steps:
10) Advance snare and distal sleeve interface past tip of endoscope
11) Partially retract endoscope (e.g. past the pylorus into the stomach)
   i) Maintain snare in position as endoscope is retracted
   ii) Take care not to dislodge or displace sleeve
12) Release snare
13) Advance snare past the distal sleeve interface
   i) Clear distal interface from snare
14) Retract snare into endoscope
   i) Take care not to dislodge or displace sleeve
15) Remove endoscope with snare
   i) Take care not to dislodge or displace sleeve
16) Attach proximal sleeve to GEJ or other anatomy
   i) Optionally attach to an interface cuff, etc.

One method for retrieval of a GI sleeve device, proximal end first, includes the following steps:
1) Release proximal sleeve attachment from tissue or attachment cuff, etc.
2) Position endoscope to view released proximal sleeve
3) Pass a grasper down the biopsy channel of the endoscope
4) Grasp the proximal sleeve
5) Retract endoscope and proximal sleeve together out the mouth
6) Remove remainder of the sleeve from the mouth

An alternate method for retrieval of a GI sleeve device, distal end first, includes the following steps:
1) With or without endoscope and/or guidewire pass a distal traction device down the sleeve, e.g. grasper, traction balloon catheter
2) Position distal traction device past the pylorus in distal duodenum, proximal jejunum or at the distal end of the sleeve
3) Engage distal traction device to grasp sleeve
4) Retract traction device and sleeve past pylorus into the stomach
5) Optionally, release and remove distal traction device
6) Release proximal sleeve attachment from tissue or attachment cuff, etc.
7) Position endoscope to view released proximal sleeve
8) Pass a grasper down the biopsy channel of the endoscope
9) Grasp the proximal sleeve
10) Retract endoscope and proximal sleeve together out the mouth
11) Remove remainder of the sleeve from the mouth.

One of the challenges in treating morbid obesity with implantable devices such as those described herein and in the prior application, and in gastric surgery in general, is avoidance of tissue erosion at the attachment points, particularly as a result of pressure necrosis. Prolonged ischemia due to excessive pressure on the gastric or esophageal wall frequently leads to pressure necrosis, which can result in detachment of the implanted device. Excessive pressure can occur at attachment points or at any interface between the tissues and an implanted device. Motion of the gastric or esophageal wall due to expansion and contraction due to stomach contents or muscular peristaltic action can create or exacerbate excessive pressure in the tissues, which may lead to ischemia, pressure necrosis and tissue erosion. Avoiding ischemia is key to preventing pressure necrosis and tissue erosion at the attachment points and any other interface between the tissues and the implanted device. Tissue, devices and device attachment in many clinical situations are designed to function in concert. For example it can be desirable to have a device which does not change the anatomy at its attachment location, allows the tissue to move with minimal resistance and is attached with an attachment means that elicits minimal foreign body reaction and imposes a minimal increase in tissue pressure. Alternatively, the device and its attachment may be constructed to elicit a controlled or measured tissue response that may increase and improve the tissues ability to retain the device and attachment means.

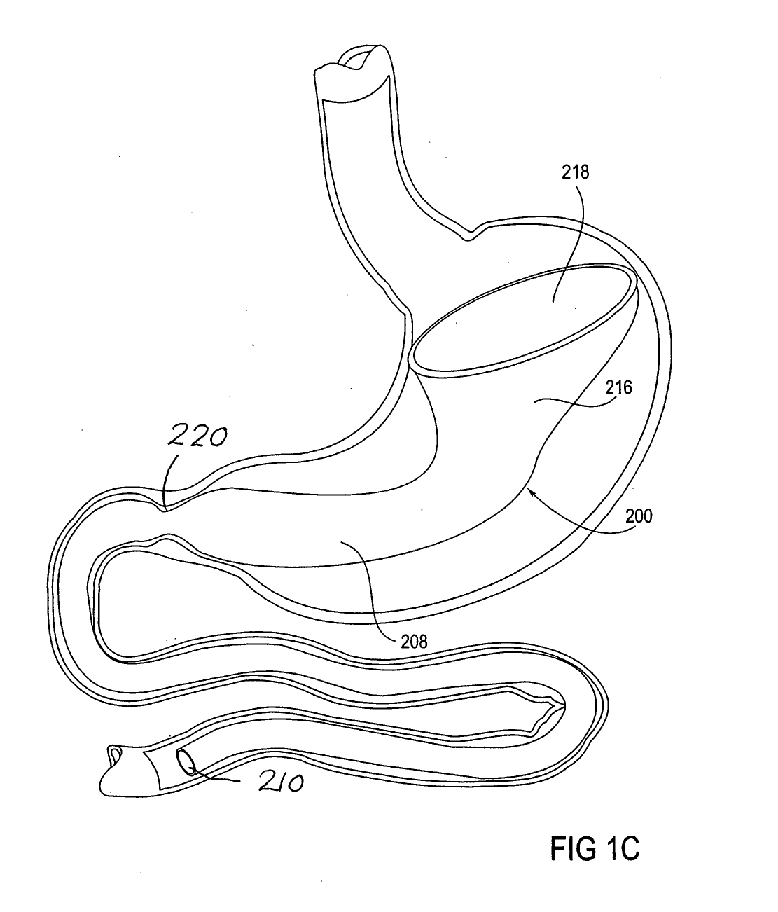

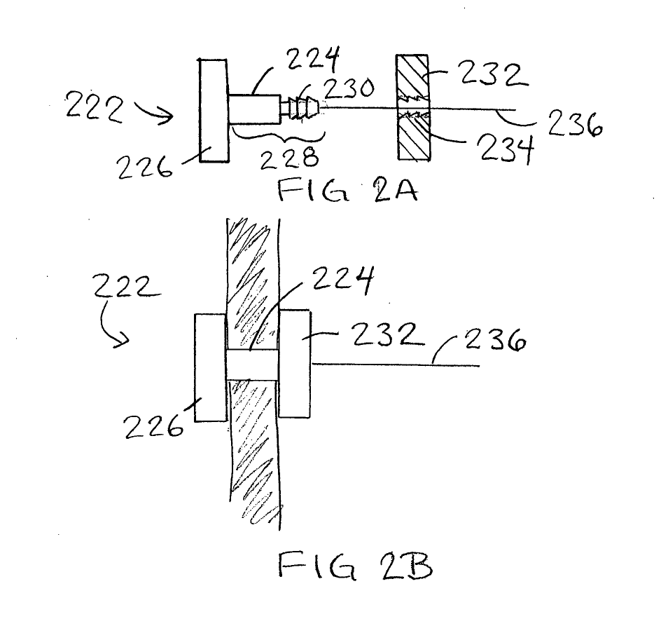

FIGS. 2A-2B show a T-tag fastener 222 with a spacer 224 to avoid excessive pressure on the tissue. The fastener 222 has a cross member or “T” 226 that is attached to a “stem” 228 at or near the mid-point of the T. For “blind” deployment of the fastener, the attachment point between the T 226 and the stem 228 can be configured with a flexible hinge to facilitate insertion through a needle or cannula. The stem 228 is constructed with a spacer 224 that may be configured as a cylindrical shoulder on the stem as shown or, alternatively, a ring or bump on the stem of the fastener may also serve as a spacer. An attachment means 230 is provided at the proximal end of the stem for attachment of a proximal cap 232. The attachment means 230 may include barbs, detents, crimp connections, screw threads, or the like, with corresponding structures or attachment means 234 on the proximal cap 232 for an easy and reliable attachment. Optionally, an elongated tail member 236 may be attached to the proximal end of the stem 228 to aid in guiding the proximal cap 232 into place on the attachment means 230 of the T-tag fastener 222. The tail 236 may be configured as a pair of elongated sutures. The tail 236 may be detachable or it may be made so that it can be cut off of the fastener after it has been placed.

Generally, the spacer 224 should be configured to limit the amount of compression applied to the gastric or esophageal wall upon deployment of the fastener 222. Where some compression is desired, the spacer distance, that is the distance along the stem from the T 226 to the proximal cap 232 after deployment, should be slightly less than the total thickness of the tissue and other structures to be attached. The spacer distance should take into account whether a single-wall transmural attachment or a double-wall plicated attachment is intended, as well as the thickness of any device structures that will be held by the fastener. In cases where it is not necessary to apply compression, the spacer distance may be greater than the total thickness of the tissue and other structures to be attached.

In an alternate embodiment of the T-tag fastener 222 of FIGS. 2A-2B, rather than using a fixed-length spacer, the attachment means 230 may be configured to allow the proximal cap 232 to be attached at different distances from the T cross member 226. This would allow the operator to select the correct spacer distance at the point of use and even to vary the spacer distance from one fastener to the next depending on tissue and device thickness at the attachment point. The correct spacer distance may be determined by imaging techniques such as fluoroscopy or ultrasound or it may be determined by using a force limiting or measuring mechanism in the fastener delivery and deployment device.

The T-tag fastener 222 of FIGS. 2A-2B can be deployed directly, for example through a plication of the gastric wall, with a needle attached to the proximal end of the tail member 236. Once the tail 236 has passed through the tissue, the proximal cap 232 is threaded onto the tail. The proximal cap 232 is then attached to the stem 228 of the fastener by holding tension on the tail 236 and pushing the proximal cap 232 until the attachment means 234 of the proximal cap engages the attachment means 230 on the stem 228. The tail 236 can then be removed from the fastener 222. Alternatively, the tail 236 can be used to attach another device to the T-tag fastener 222. The T-tag fastener 222 of FIGS. 2A-2B can also be deployed blindly, for example for transmural attachment through the gastric wall. The fastener 222 is inserted through a needle or cannula by pivoting the T 226 so that it is approximately parallel to the stem 228. The needle or cannula is used to pierce through the tissue to be attached; then the T 226 is pushed out of the cannula on the far side of the tissue using a pusher rod or tube that extends through the cannula. The needle or cannula is withdrawn and the T 226, which is now approximately perpendicular to the stem 228, is snugged up to the back surface of the tissue with a little tension on the tail member 236. After the needle is withdrawn, the proximal cap 232 is threaded onto the tail 236. The proximal cap 232 is then attached to the stem 228 of the fastener by holding tension on the tail 236 and pushing the proximal cap 232 until the attachment means 234 of the proximal cap engages the attachment means 230 on the stem 228. The tail 236 can then be removed from the fastener 222. The diameter of the spacer 224 and/or the stem 228 between the T 226 and the proximal cap 232 can be minimized to reduce compression on the surrounding tissue or, alternatively, the spacer 224 and/or stem 228 can be configured to seal the puncture through the tissue. FIG. 2B shows the T-tag fastener 222 of FIG. 2A deployed through the gastric wall.

A gastrointestinal sleeve, a mounting ring or other device may be attached directly to the gastric wall using several of the T-tag fasteners as rivets. Alternatively, the stem and/or the proximal cap may be configured with a ring, a hook or the like for attaching another device to. As another alternative, the suture tails may be used for tying a device to the fasteners.

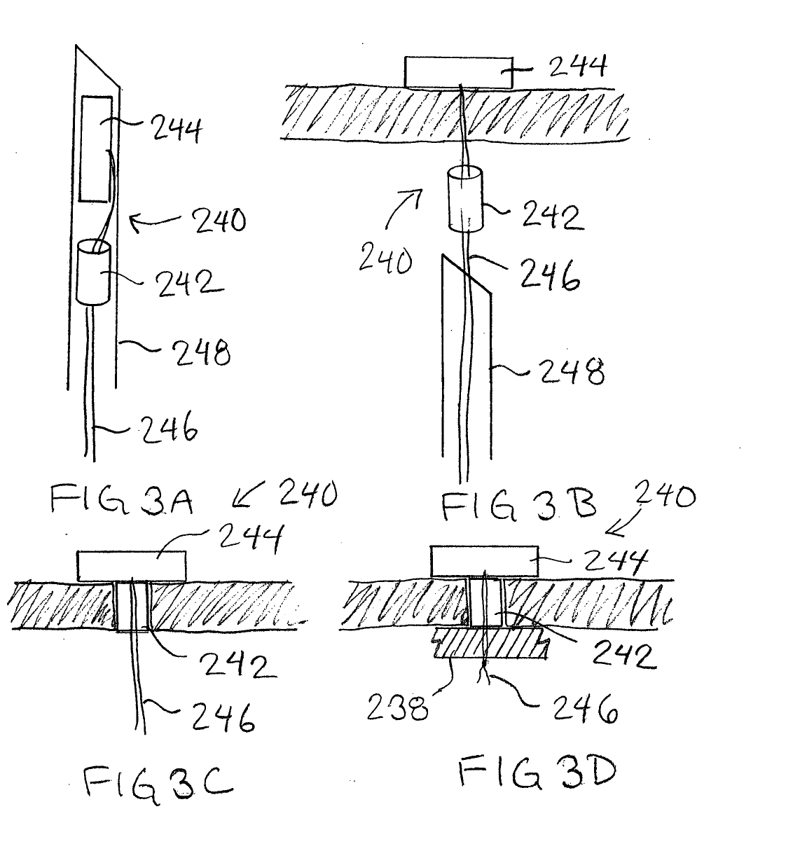

FIGS. 3A-3D show another T-tag fastener 240 with a spacer 242 to avoid excessive pressure on the tissue. In this case the T-tag fastener 240 is specially configured for low-profile blind deployment through a needle or cannula 248. The cross member or T 244 is attached at or near its mid-point to a flexible stem 246, which in the example shown is configured as a pair of elongated sutures. A sliding spacer 242 or standoff with a tubular configuration is threaded onto the sutures 246. The T-tag fastener 240 is prepared for insertion as shown in FIG. 3A by inserting it into the delivery cannula 248 with the T 244 at the distal end, followed by the spacer 242 in a tandem configuration. The sutures 246 extend proximally through the delivery cannula 248. The tandem configuration provides a low profile for delivery through the cannula 248. The fastener 240 may be loaded into the delivery cannula 248 in either an antegrade or retrograde direction, depending on what is most convenient and economical.

The low profile provided by the tandem configuration of the T member 244 and spacer 242 is desirable to minimize the size of the needle or cannula 248 needed to deliver the fastener 240. This is important not only for minimizing the size of the tissue puncture, but also to reduce the amount of force needed to deliver and deploy the fastener 240 through an endoscope. The delivery needle or cannula 248 will preferably be 17 gauge or smaller, more preferably 19 gauge or smaller.

The T-tag fastener 240 is typically deployed as a blind fastener, for example for transmural attachment through the gastric wall. The delivery cannula 248 is used to pierce through the tissue to be attached; then the T 244 is pushed out of the cannula 248 on the far side of the tissue using a pusher rod or tube that extends through the cannula 248. The needle or cannula 248 is withdrawn and the T 244, which is now approximately perpendicular to the sutures 246, is snugged up to the back surface of the tissue with a little tension on the sutures 246, as shown in FIG. 3B. The sliding spacer 242 is pushed out of the delivery cannula 248 with the pusher rod or tube and slid distally along the sutures until it contacts the T member 244, as shown in FIG. 3C. Several T-tag fasteners 240 can be inserted transmurally in this manner around the gastroesophageal junction or elsewhere in the gastrointestinal system using a flexible gastroscope or the like. The sutures 246 can then be used to attach a gastrointestinal sleeve, a mounting ring or other device 238 to the gastric wall, as shown in FIG. 3D. The spacers 242 prevent excessive pressure on the gastric wall that could lead to ischemia and eventually to tissue necrosis.

Spacers illustrated in FIGS. 2 and 3 can also reduce pressure on tissue due to their diameter. As described in the parent application, Ser. No. 10/698,148, T fasteners perform best when forces are perpendicular to the T member, or along the axis of the tail and spacer. However, there is frequently a component of force perpendicular to this preferred direction. In this case the increased diameter of the spacer can serve to reduce tissue pressure in this direction.

When placing T-tag fasteners or other fasteners in the region of the GEJ, it is important to avoid other anatomical structures in the vicinity of the stomach and esophagus. One method for this is to create a safe space behind the GEJ for deploying the fasteners. One method to accomplish this is described in the parent application, Ser. No. 10/698,148. Alternatively, one can take advantage of the fact that the proximal stomach generally lies just below the diaphragm when the patient is in a head-up position. Space will be created between the stomach and diaphragm into which transmural fasteners can be safely placed. This safe space can be increased by having the patient inhale deeply while in a head-up position to push the stomach down with the diaphragm, then exhale to lift the diaphragm up off of the stomach. Preferably, the fasteners 250 will be delivered parallel to the diaphragm 252, as shown in FIG. 4, though other orientations are possible. FIG. 4 also shows an optional stomach traction device 254 deployed through the working channel of an endoscope 256 that helps to facilitate safe deployment of the fasteners 250 in the GEJ region. The traction device 254 can be used to retract the gastric wall laterally 254A and/or distally 254B to create a safe place for deployment of the fasteners 250. Due to anatomic variations and pathology, the position of the diaphragm relative to the stomach and GEJ should be confirmed prior to using this technique.

Alternatively or in addition, pneumoperitoneum can be used to create a safe space around the stomach and esophagus. Pneumoperitoneal pressure will tend to collapse the stomach away from other surrounding organs and would be balanced by the pressure used to endoscopically insufflate the stomach for improved visualization and access.

Other tactics to avoid other anatomical structures in the vicinity of the stomach and esophagus include the use of imaging techniques such as fluoroscopy, esophageal ultrasound imaging, external ultrasound imaging and/or Doppler imaging when placing fasteners. Alternatively or in addition an “endoscopic compass” can be used to provide a reference for orienting the endoscope when using fastening devices. A small magnetized needle (i.e. a compass needle) is placed near the distal end of the endoscope where it can be viewed by the operator through the endoscope. A magnet is placed on the patient to provide a reference point for the compass, for example the reference magnet can be placed on the patient's back directly over the spine. The compass needle will point toward the reference magnet on the spine. Using the compass needle as a reference, the operator will be able to avoid inadvertently puncturing the aorta, which lies directly posterior to the esophagus.

The concept of the Veress needle can be adapted for avoiding puncturing other anatomical structures in the vicinity of the stomach and esophagus during endoscopic attachment of devices near the GEJ. A Veress needle is a needle equipped with a spring-loaded obturator that is often used for insufflation of the abdomen in laparoscopic surgery. A long, flexible device with a needle at the distal end and a spring-loaded obturator within the needle would be used to safely puncture the gastric or esophageal wall. Once the needle has passed through the wall, the spring-loaded obturator advances automatically to avoid damage to any surrounding tissues. A delivery cannula can be advanced over the needle and the needle can be exchanged with a fastener delivery device. Alternatively, this concept can be adapted directly into the fastener delivery device. A T-tag fastener or the like would be spring-loaded into the lumen of a delivery cannula so that it would be ejected out of the lumen immediately after the cannula has traversed the gastric or esophageal wall.

Another method for avoiding deploying fasteners into the aorta would involve a small diameter needle with a flow detector (e.g. a Doppler flow sensor) or pressure detector for detecting blood flow or blood pressure. Alternatively, a flow detector or pressure detector can be mounted on a separate guidewire inserted through the needle. The flow detector can be used to detect blood flow before the wall of the aorta is punctured. Alternatively, if backflow of blood or blood pressure is detected, indicating that the needle has punctured the aorta, the needle will be withdrawn and a fastener will not be delivered at that site. The small diameter puncture in the aorta should heal without complications.

Alternatively or in addition, the organs and other anatomical structures in the vicinity of the stomach and esophagus can be protected during endoscopic attachment techniques by using a depth stop on the needle or delivery cannula to prevent it from penetrating farther than necessary to traverse the gastric or esophageal wall. Examples of fastener delivery devices with a depth stop to protect nearby organs and structures are described in U.S. provisional patent application 60/569,442.

One method for placing an implantable device within a patient's body has been described as a “parachuting” technique. In this technique, multiple elongated sutures are sewn through the tissue where the device is to be implanted with the ends of the sutures extending out of the patient's body. The ends of the sutures are passed through a sewing ring or similar structure on the device while the device is still outside of the patient's body, then the device is parachuted or slid into place along the sutures. The device is typically secured in place by knotting the elongated sutures with the help of a knot pusher or similar device and then the sutures are cut off close to the knots. U.S. provisional patent application 60/534,056 describes a variation of this method for implanting a device within a patient's digestive tract using T-tag fasteners. Alternatively, suture locks such as those described in U.S. Pat. No. 4,235,238 or those used in the BARD Endocinch system can be used to secure the suture prior to cutting.

When parachuted into place along the sutures, the device may be folded or compressed to pass through the esophagus or through a delivery tube placed in the esophagus. When using this parachuting technique it is desirable to minimize the friction between the device and the sutures. This can be done by using a low friction material or a low friction coating on the sutures and/or the device. This is also done by dimensioning and/or orienting structures, e.g. holes, to guide the parachuted device to reduce friction.

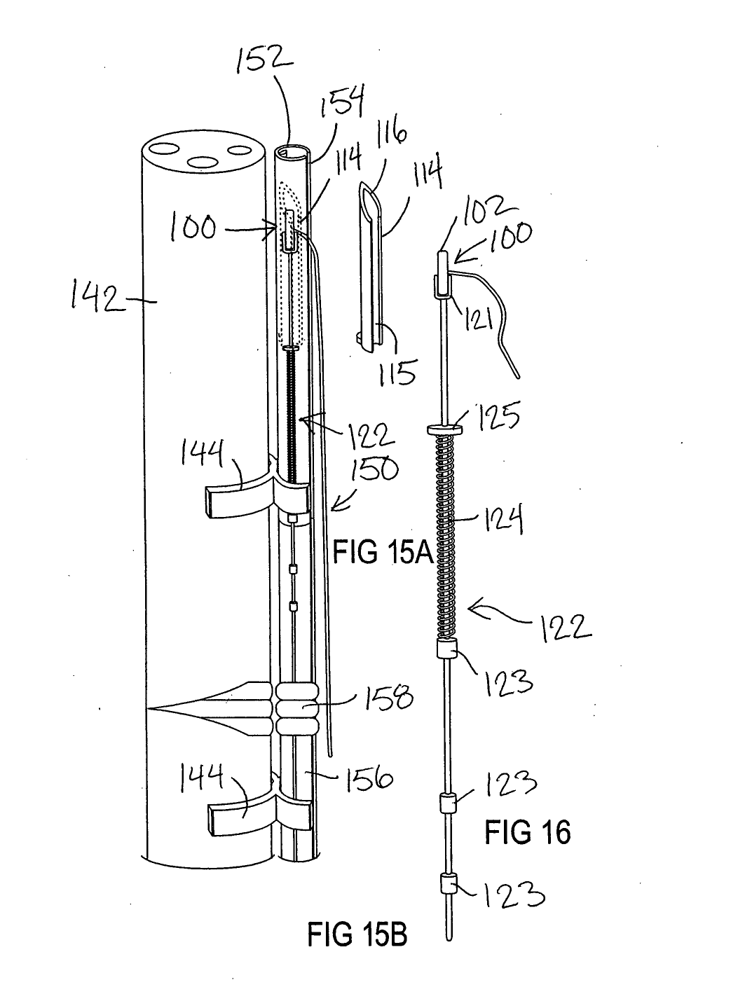

FIG. 5 shows an implantable device 120 being implanted at the GEJ using a parachuting technique. One method of using a fastener delivery device 150 (such as the example shown in FIG. 15B) for placement of an implantable device 120 by the parachuting technique includes the following steps:
1) attach delivery device 150 to scope 142;
2) position scope 142, e.g. in stomach;
3) aim scope 142 and load and position T-tag fastener 100 for delivery;
4) place scope 142 against tissue, optionally use vacuum or grasper through biopsy channel to hold tissue;
5) advance pusher 122 to have penetrating cannula 114 pierce tissue, and then deliver the T member 102 of the T-tag fastener 100;
6) retract pusher 122 and penetrating cannula 114 and secure suture tail 104 in known position to prevent tangling;
7) repeat steps 3-6 to deploy multiple fasteners 100, typically 6-8 times;
8) remove scope 142, suture tails 104 should extend parallel from scope and be at exit orifice;
9) identify and organize suture tails 104, confirm that tails remain untangled;
10) thread suture tails 104 through device 120 at locations corresponding to positions of T-tag fasteners 100 in tissue as shown in FIG. 5;
11) slide device 120 along suture tails 104 into position;
12) secure device 120 by completing attachment according to the design of the T-tag fasteners 100;
13) trim excess material from suture tails 104.

Alternatively, the device 120 may be partially parachuted into place, meaning that 2-4 parachute sutures are used to slide the device 120 into position with the proper orientation. Then additional fasteners, for example T-tag fasteners, are delivered to complete the attachment of the device 120 to the tissue.

If suture tails are delivered through a closed lumen (e.g. in or attached to an endoscope), the lumen must be removed from around the suture tails before a device can be parachuted over the sutures if the device is too large to pass through the lumen. This can present a challenge related to maintaining the organization of the suture tails and preventing confusion, crossing, winding and/or tangling of the suture tails. If T-tag fasteners and their suture tails are passed externally e.g. through an external lumen with a longitudinal slot or in a non-enclosed rail type system, the suture tails can be managed external to the lumen used to place the T-tag fasteners and external to the scope. This facilitates manipulation of the scope, simplifies scope exchanges and simplifies suture tail management.

Suture tail management external to the scope or an enclosed lumen can be combined with suture holders external to the patient, similar to those used for parachuting replacement heart valves into place. Snugging the sutures as described above is simpler when the suture tails are external to the scope, as is avoidance of crossing, winding and/or tangling of the suture tails. Suture holders, such as slots, clamps or clips, can be combined with a mouth guard for organizing the sutures during a peroral parachuting procedure.

One aspect of suture tail management is that it must happen from one end of the system to the other. Therefore, the method and apparatus must address this issue. For example, after placement of a T-tag fastener, a slight tension on the suture tail can hold the suture against the wall of the lumen or in a straight position where it is less likely to tangle. Apparatus can include means to maintain tension while allowing scope movement and manipulation, e.g. tension from a long soft spring, an elastic band or a spring-loaded reel.

Sometimes, when performing an endoscopic procedure, an overtube is used to line the esophagus and protect it from damage due to insertion and manipulation of the endoscope and related tools and devices. Other practitioners prefer to avoid the use of an overtube. In either case, it may be desirable to secure an implant being parachuted down the esophagus in a collapsed, folded or otherwise reduced configuration. A major issue when parachuting a device into place is friction between the device and the sutures, and collapsing or folding the device may exacerbate the problems with friction.

The following method is intended to reduce the problems with friction between the device and the sutures when parachuting a device through the esophagus. The method allows the device to be parachuted through the esophagus in a folded configuration, while it also allows the sutures to pass through the device while it is in an unfolded position. In addition, the method allows the sutures to be pulled through the device one at a time, which further reduces the problems with friction. This method can be used, for example, with the t-tag and/or t-tag delivery systems described herein.
1) Place fasteners (e.g. 6-10) in or through gastric wall with suture tails extending out through the patient's mouth; the sutures should have a length that is about 100-140 cm longer than required to exit the mouth;
2) thread suture tails through the device to be parachuted into place, e.g. an implant mounting ring;
3) slide the device down the sutures until it is just outside of the patient's mouth, with 100-140 cm of suture extending beyond the device;
4) fold or collapse the device and secure it in the collapsed position, e.g. with a removable sack or tied with a suture;
5) slide the device through the esophagus or the scope overtube (the device is not slid down the sutures, but instead the sutures are allowed to move with the device into the esophagus with the ends of the sutures remaining outside the patient);
6) once the device is through the esophagus and inside the patient's stomach, the device is release from its collapse position, and any restraining device that was used is removed perorally;
7) while controlling the device (e.g. with a grasper), and preferably under direct vision, pull each suture through the device until all the slack is removed and the device is at or near its intended position in the stomach;
8) position and secure the device in its intended position in the stomach.

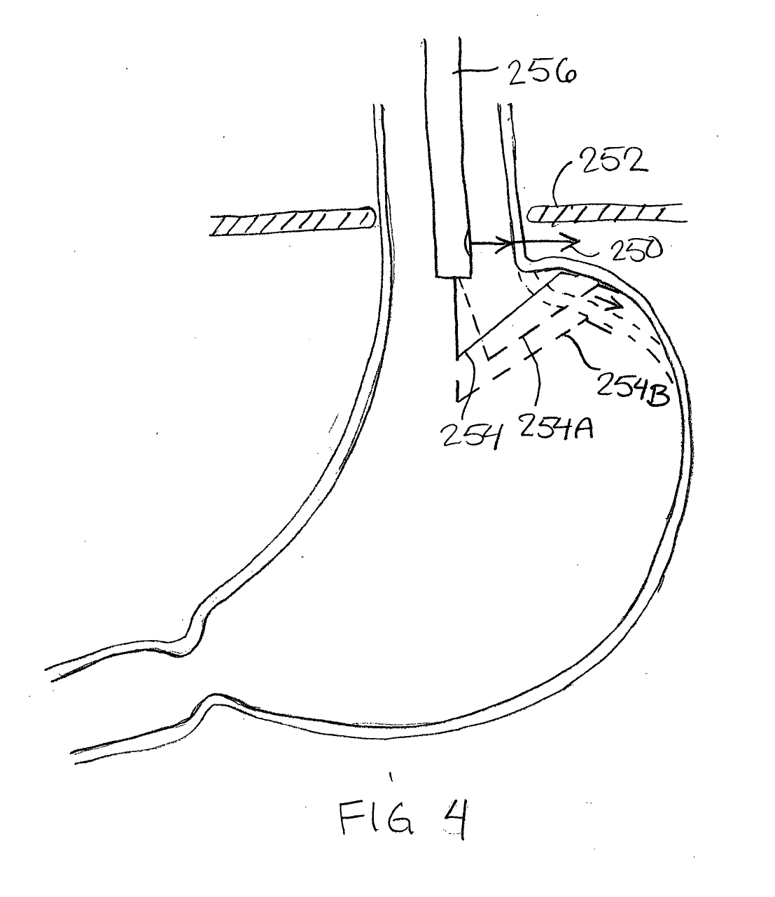

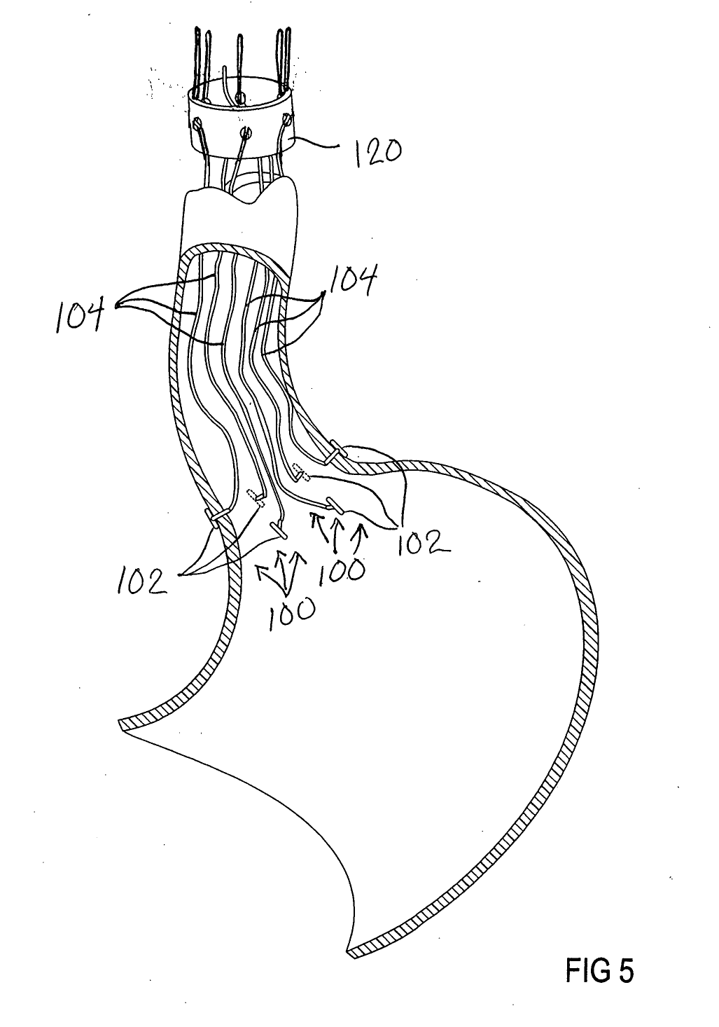

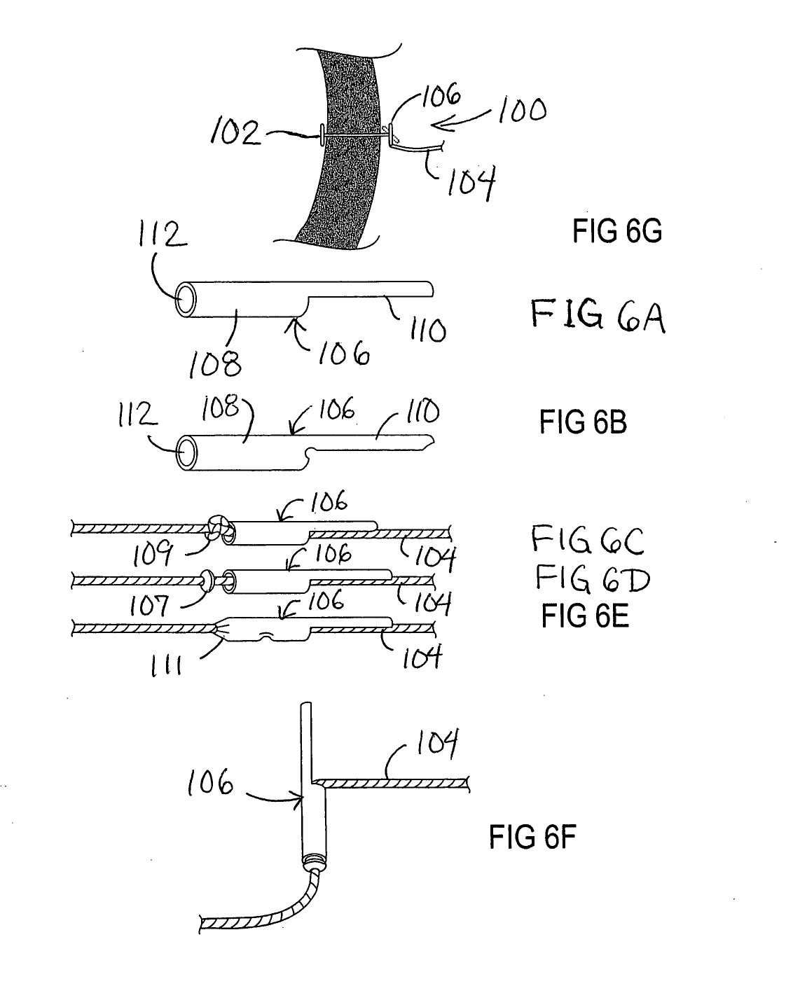

FIGS. 6A-6G illustrate a dual-headed T-tag fastener 100 that is especially adapted for attaching devices that are parachuted into place within a patient's digestive tract. Alternatively, the dual-headed T-tag fastener 100 can also be used to attach devices that are not parachuted into place as well as attaching tissue to tissue. The T-tag fastener 100, which is shown being deployed in FIG. 6G, has a primary T member 102 that is pivotally attached near its center to the end of an elongated suture 104. The primary T member 102 has an undeployed position wherein the primary T member 102 is approximately parallel to the body of the elongated suture 104 and a deployed position wherein the primary T member 102 is approximately perpendicular to the body of the elongated suture 104. A secondary T member 106 is pivotally attached near its center to the body of the elongated suture 104 at a position spaced apart from the primary T member 102. The secondary T member 106 has an undeployed position wherein the secondary T member 106 is approximately parallel to the body of the elongated suture 104 so that it presents a low profile so that a device can be slid in place along the elongated suture 104 and over the secondary T member 106 and a deployed position wherein the secondary T member 106 is approximately perpendicular to the body of the elongated suture 104. The fastening gap, that is distance between the primary T member 102 and the secondary T member 106, may be fixed or, optionally, the secondary T member 106 may be slidable along the body of the elongated suture 104 to adjust the fastening gap.

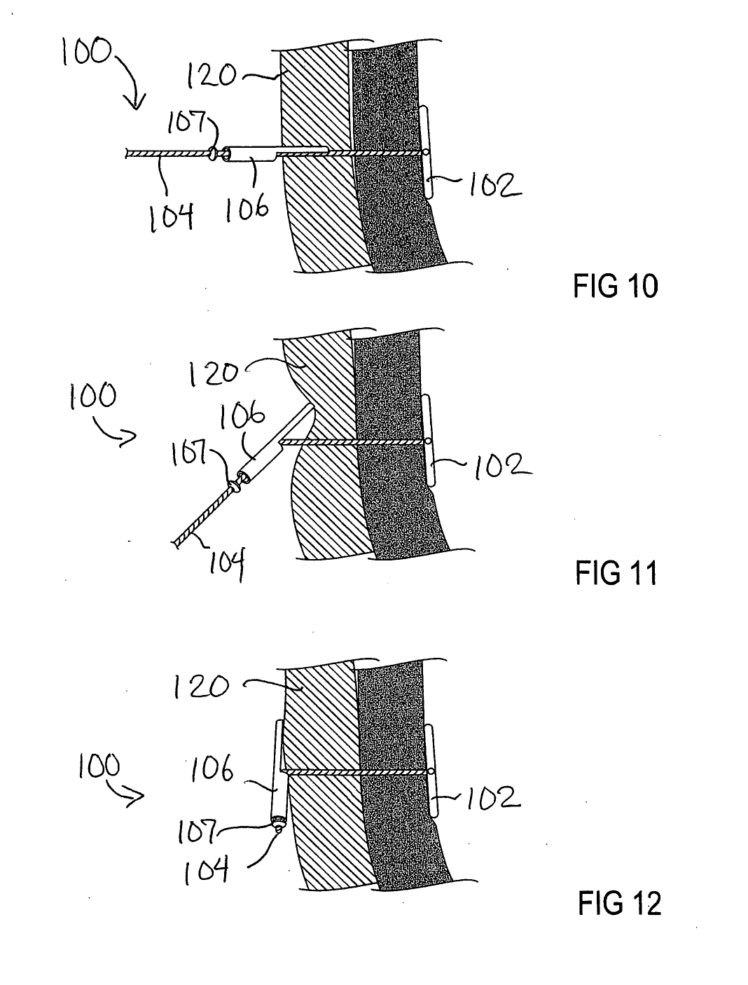

FIGS. 6A-6F are detail drawings of the secondary T member 106 of the T-tag fastener 100 of FIG. 6G. FIGS. 6A-6B show two variations of the secondary T member 106 alone. FIGS. 6C-6E show three variations of the secondary T member 106 in the undeployed position wherein the secondary T member 106 is approximately parallel to the body of the elongated suture 104 so that it presents a low profile so that a device can be slid in place along the elongated suture 104 and over the secondary T member 106. Various securing members, including a stopper member 107 (FIG. 6D), knot 109 (FIG. 6C), crimp 111 (FIG. 6E) are shown. FIG. 6F shows the secondary T member 106 in the deployed position wherein the secondary T member 106 is approximately perpendicular to the body of the elongated suture 104. In the embodiment shown, the secondary T member 106 is preferably constructed from a rigid tubular material, for example NiTi, Ti or stainless steel tubing. A first end 108 of the secondary T member 106 is tubular in configuration and the body of the elongated suture 104 passes through the lumen 112 of the tube. A second end 110 of the secondary T member 106 is cut away around approximately 60-180 degrees of its perimeter along one side to allow the secondary T member 106 to pivot or swivel relative to the body of the elongated suture 104 as shown in FIG. 6F. The secondary T member 106 is pivotally attached to the body of the elongated suture 104, for example by a stopper structure, a knot in the suture 104, crimping, adhesive or other attachment means. Attachment of the T member 106 to the suture will optionally prevent motion of the T member in either direction. Restriction of motion in the proximal direction will enable the T member to function to hold a structure in place, as shown in FIG. 12, while restriction of motion in the distal direction will facilitate passage of the T member 106 through a structure as shown in FIG. 10.

In some embodiments, a stopper member 107, or other securing means such as an adhesive, crimp or knot may be used alone or in combination to create a tapered or gradual proximal transition, which may facilitate passage of the secondary T member 106 through other structures as shown in FIG. 10. Optionally, the secondary T member 106 may be slidable along the body of the elongated suture 104 in order to adjust the fastening distance. In this case, a secondary securing means would be applied to the T member 106 once it is in place. This could be a knot, crimp, adhesive, stopper member 107, or other securing means known in the art. In some embodiments more than one stopper will be appropriate, e.g. one to prevent distal motion and one to prevent proximal motion.

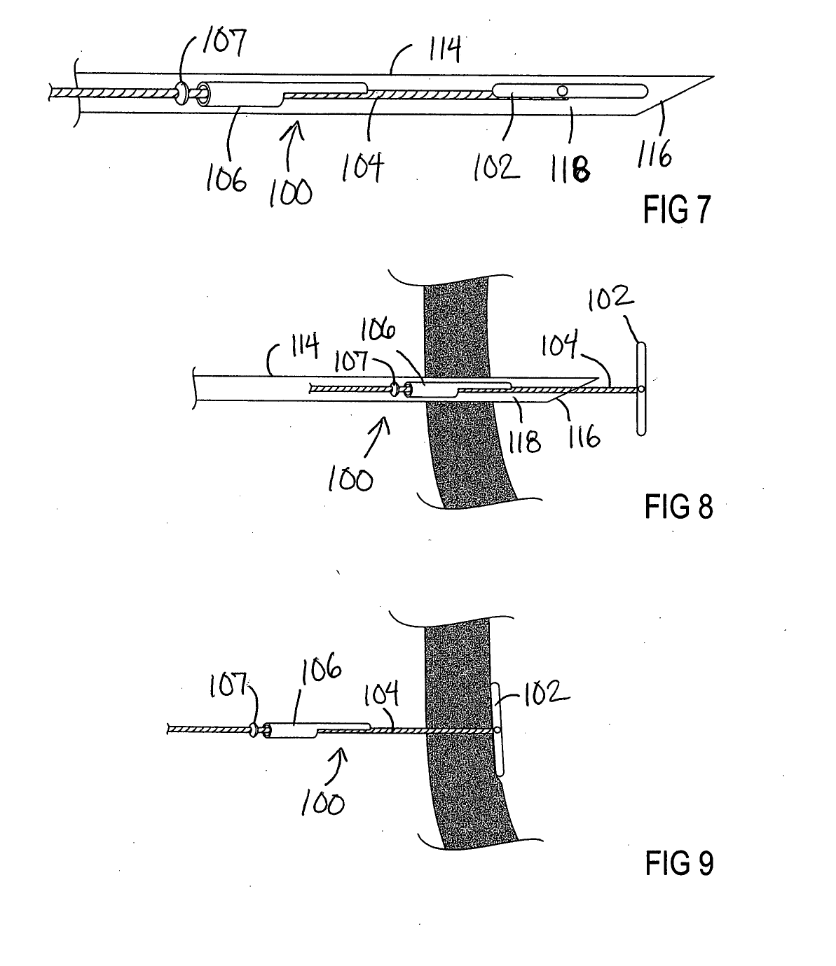

FIGS. 7-12 show the steps for deploying a dual-headed T-tag fastener 100 of the type shown in FIGS. 6A-6G. FIG. 7 shows the T-tag fastener 100 positioned within the lumen 118 of a delivery cannula 114 with the primary T member 102 and secondary T member 106 in the undeployed position. The elongated suture 104 extends through the lumen 118 and out the proximal end of the delivery cannula 114. The delivery cannula 114 can be part of a fastener delivery device, which is explained in more detail below. The delivery cannula 114 has a sharpened distal end 116 that is used to penetrate the tissue that is to be fastened, for example the gastric or esophageal wall in the vicinity of the GEJ. The primary T member 102 is ejected from the delivery cannula 114 and deployed behind the tissue. FIG. 8 shows the T-tag fastener 100 with the primary T member 102 deployed. The delivery cannula 114 is removed by withdrawing it with the enclosed secondary T member 106 through the tissue and then further withdrawing the cannula 114 from the proximal end of the suture 104. Then the primary T member 102 is snugged against the tissue with a little tension on the suture 104 in preparation for parachuting or otherwise attaching a device into place. Alternatively, the delivery cannula 114 can be passed through two or more layers of tissue, two or more devices or a combination of layers of device and tissue to achieve the configuration similar to that shown in FIG. 10. FIG. 9 shows the T-tag fastener 100 after the delivery cannula 114 has been removed. After a sufficient number of fasteners have been placed in the tissue, the device to be implanted 120 is parachuted into place by passing the sutures 104 through the device 120 and sliding the device 120 down the sutures 104 until it is in contact with the tissue. The device 120 is slid over the secondary T member 106, which is still in its undeployed position. FIG. 10 shows the T-tag fastener 100 after a device 120 has been parachuted into place. Tension is applied to the elongated suture 104 (and, optionally, a pushing force is applied to the device 120) to provide clearance for the secondary T member 106 to rotate or swivel to the deployed position. FIG. 11 shows the T-tag fastener 100 with the secondary T member 106 being deployed. The tension of the suture 104 is released to allow the secondary T member 106 to fully deploy and the suture 104 is trimmed proximal to the secondary T member 106. FIG. 12 shows the T-tag fastener 100 fully deployed. Optionally, a lateral force may be applied to the secondary T member 106 to assist deployment.

The fastening gap determines the tension on the suture 104 and hence the pressure on the tissue exerted by the primary T member 102 and the implanted device 120. This would ideally be sufficient to create a seal (optionally in conjunction with ingrowth) while applying minimal force to the tissue. Optionally, this gap can be variable and or changeable as described herein.

Other configurations of fasteners and fastener delivery devices known in the art can be used in conjunction with the present invention. For example, U.S. Pat. No. 4,235,238 describes various fasteners and endoscopic fastener delivery devices for use in the gastrointestinal system. Other attachment and /or parachuting approaches can be used with these dual t-tag fasteners to secure devices, for example, to plications.

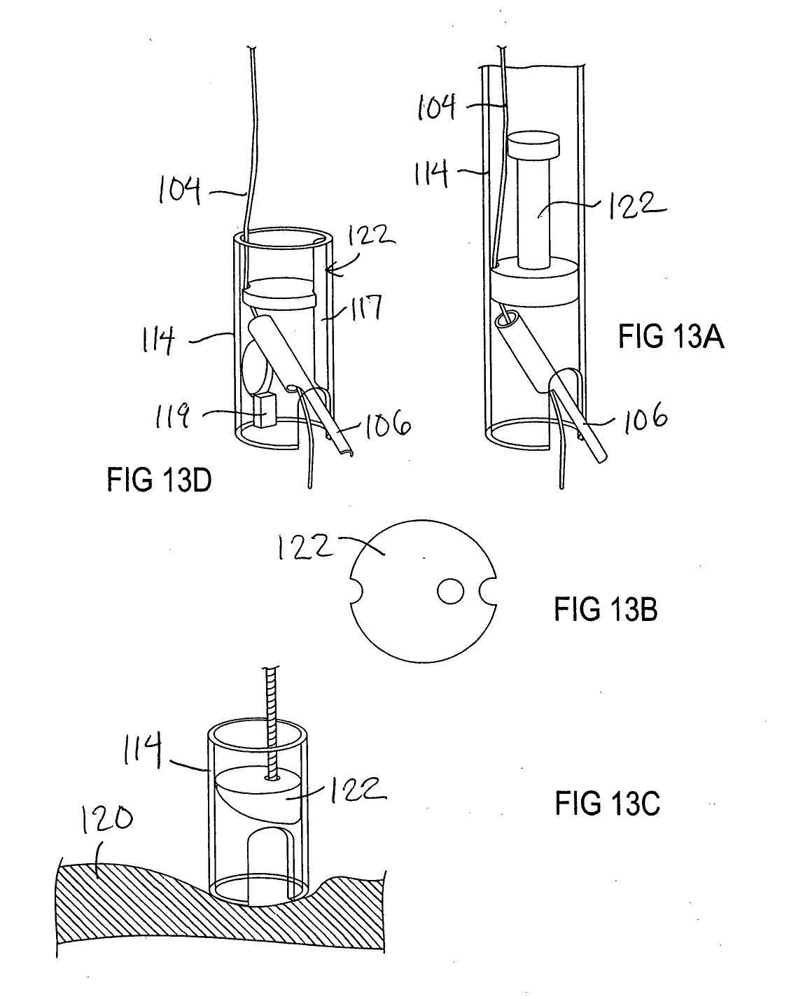

FIGS. 13A-13D are detail drawings showing variations of a delivery device for deploying a T-tag fastener 100 of the type described above after the primary T member 102 has been positioned. FIGS. 13A and 13D shows a pusher device 122 that is used for deploying the T-tag fastener 100 through the delivery cannula 114.

The delivery device can include means to apply force to the implanted device 120, which may facilitate and/or cause swiveling or rotation of the secondary T member 106. In some embodiments, this device can also cut the suture 104.

The pusher causes the secondary T member 106 to swivel as it advances distally. The pusher may include a window to allow the first end 108 of the secondary T member 106 to swivel. This window should be preferably open at the distal end. Optionally, the pusher may include another window to allow the second end 110 of the secondary T member 106 and the suture 104 to swivel. Optionally, the window can also be used to cut the suture as shown in FIG. 13D where the proximal end of the suture can pass out through the window where advancing the pusher past the window can cut the thread An optional anvil 119 to cut/trim the suture is shown in FIG. 13D where slots in the pusher locate the pusher and suture relative to the anvil so advancing the pusher past the anvil will cut the suture.

The distal end of delivery device may be used to apply force to the implanted device 120, as shown in FIG. 13C, to assist in deployment of the secondary T member 106. This can help create room for the secondary T member 106 to swivel. This can be particularly helpful when the secondary T member 106 is in a fixed position relative to the primary T member 102 and it is desired that the two T members apply tension to the device and tissue being attached.

One example of a pusher, as shown in FIG. 13C, would include a distal end that has a swivel-inducing angle. This would preferably include a delivery device body incorporating means to key the orientation of the pusher so it guides the swiveling of the secondary T member 106 to any existing window in the delivery device. Such a keying structure is 117 in FIG. 13D.

One method of deploying the secondary T-tag 106 utilizes the following steps:
1) Apply tension to suture 104.
2) Apply push to delivery device body 114 to create room for the T member 106 to swivel.
3) Apply push to delivery device pusher 122; Secondary T-tag 106 swivels partly.
4) Allow delivery device body 114 to retract slightly relative to suture 104 and release tension on suture 104; Secondary tag 106 rotates fully.
5) While maintaining the position of the delivery device pusher 122 relative to the implanted device 120, pull back on delivery device body. Alternatively, the pusher may be advanced toward or into the delivery device. The key is relative motion of the two structures and maintenance of the pusher in contact with the T member 106 and implanted device 120. This cuts suture 104, which has preferably been forced out the proximal end swivel window of the delivery device pusher and swiveling of the secondary T member 106. The delivery device body may incorporate means to laterally displace the delivery device pusher to facilitate positioning of the suture 104 for trimming/cutting.

Delivery cannula devices/systems (hereinafter delivery cannula) can be configured for the delivery of multiple T-tag or other fasteners. In particular these delivery cannulae allow placement of multiple T-tags (or in the case of a dual-headed T-tag fastener, primary T members) through a layer of tissue and/or a device while facilitating the management of multiple fastener suture tails.

A delivery cannula can include some or all of the following components, which will be described in more detail below in relation to specific embodiments of delivery cannulas:
penetrating cannula to penetrate the tissue and delivery the T member of the T-tag;
transit cannula to delivery the T-member of the T-tag to and into the proximal end of the penetrating cannula;
loading cannula to load or position the T member of the T-tag at and into the proximal end of the transit cannula;
garage or protective cannula to provide a shield into and out of which the penetrating cannula can be advanced and retracted when appropriate;
pusher to perform any or all of the functions related to advancing a T-tag through the loading cannula, through the transit cannula, through the penetrating cannula and expelling the T-tag out of the penetrating cannula.

In a basic delivery cannula embodiment, the first components are a single elongated hypodermic tube with a sharpened distal tip as the penetrating cannula and a wire or rod as the pusher. This type of device generally delivers a single fastener before being withdrawn to clear the suture tail from the tube. A more complicated delivery cannula is similar to the above, but incorporating a longitudinally slotted hypotube. This allows the tail/suture of the T-tag fastener to be external to the hypotube and allows a smaller diameter hypotube as well as other suture/tail handling advantages. In this case the above-mentioned penetrating, transit and loading cannulas are embodied in the single cannula.

In all the delivery cannulas, the penetrating cannula must be movable relative to the tissue through which it penetrates for T-tag fastener delivery. In a basic delivery cannula, the penetrating cannula will move in conjunction with the transit cannula and loading cannula. In more complex delivery cannulas, the penetrating cannula will move relative to the transit cannula and/or loading cannula.

The delivery cannula can be configured to be used:
1) within the biopsy channel of an endoscope;
2) attached to the exterior of an endoscope;
3) as a stand alone device with a separate means of aiming/visualization.

The delivery cannula should include means to keep the penetrating cannula point from inadvertently damaging tissue or the device through which it is delivered. For example, a Varess needle style obturator or other obturator can be used. The obturator must be removed to deliver the T-tag fastener through the lumen of the penetrating cannula. An external needle protector, or garage, may also be used, which has the advantage that it would not have to be removed for T-tag fastener delivery. A slotted garage could have additional advantages for T-tag fastener delivery. A penetrating cannula that is spring loaded within a garage where it only exits the garage under the impetus of a pusher in preparation to penetrating tissue would also have certain advantages.

Retracting the penetrating cannula into the biopsy or instrument channel of the endoscope will protect the tissue from inadvertent damage, but not the lining of the instrument channel. To protect the biopsy channel, the penetrating cannula could be retracted within the transit cannula or into a structure (garage) located at the juncture of the penetrating cannula and the transit cannula.

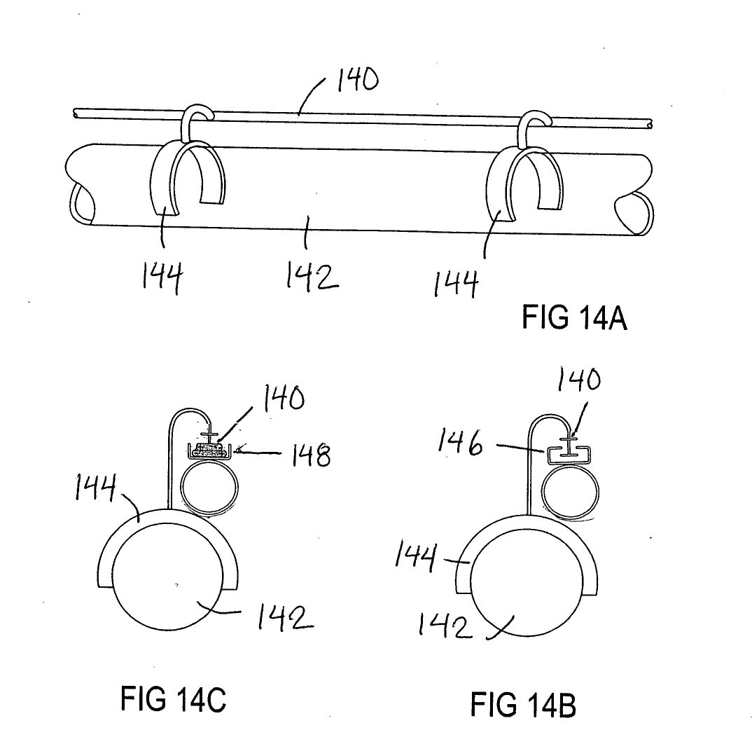

As an alternative to the slotted hypotube previously mentioned, a magnetic or mechanical rail system can be used in place of or in combination with the transit cannula. In this case, the pusher captures the primary T member 102 for delivery to the penetrating cannula. The pusher is magnetically or mechanically coupled to the transit cannula. FIGS. 14A-14C show possible configurations for a rail-mounted delivery device for deploying a T-tag fastener mounted on the exterior of a flexible endoscope. FIG. 14A shows a rail 140 mounted to the exterior of a flexible endoscope 142 using a plurality of mounting clamps 144. In one embodiment, the rail 140 may be configured as a slotted tube. Alternatively, the rail 140 of FIG. 14A could incorporate a mechanical coupling 146 as shown in FIG. 14B or magnetic coupling 148 as shown in FIG. 14C. A delivery cannula, such as described below, will be slidingly mounted to the rail 140 using one of the coupling mechanisms described.

Similarly, a smaller diameter, short length slotted hypotube transit cannula can be used with a monorail T capturing pusher as a means to transfer the T-tag fastener to the penetrating cannula. In this context “monorail” refers to a short distal coupling section such as those used to couple a monorail or rapid exchange catheter to a guidewire. In this case, the monorail transit cannula, such as shown in FIG. 22, could be coupled to a rail 140 as in FIGS. 14A-14C. With this configuration, the transit cannula, rather than extending the full length of the delivery cannula, would be a short length and would move from a position at the distal end of the loading cannula to a position at the proximal end of the penetrating cannula. This short length transit cannula length could be approximately half to two or three times the length of the T member. The term monorail T capturing pusher refers to a pusher alone or in combination with the transit cannula.

Having elongated suture tails extending out of a patient's mouth (or other orifice) with an associated need to pass devices over the suture tails can be cumbersome if standard “exchange length” techniques are applied. A monorail style device could be used through an internal or external endoscope lumen or independent of the endoscope. This type of design allows control of the T member in a short slotted cannula while the majority of the length of the suture tail would be external to an elongated transit cannula. This may also provide an easier path for the long suture tail as when an elongated slotted cannula might not maintain a slot free of obstruction when the endoscope was subjected to flexion.

If the monorail portion of the device extends out of the lumen of the endoscope, the monorail portion can optionally be of sufficient length to partially remain within the endoscope lumen to provide improved support and manipulation capability.

A two-channel endoscope can be used to deploy a series of T-tag fasteners. In one method of using such a 2-channel endoscope the T-tag fasteners are delivered through the first channel of the scope. The distal T members of the fasteners, individually or collectively, are placed outside the distal end of the scope. A delivery device is placed in the second channel and positioned near the distal tip of the scope. A capture/pusher device is passed through the first delivery cannula and a single distal T member is captured and drawn into the delivery cannula. The penetrating cannula of the delivery device is preferably slotted. The delivery device is used to deploy a series of T-tag fasteners into the tissue in the manner described above. The steps of T member capture by the pusher, drawing into the delivery cannula and deployment are repeated for each T fastener. As the endoscope is removed from the patient, the multiple suture tails of the T-tag fasteners are drawn out of the distal end of the first channel of the endoscope. The suture tails should be long enough to extend out of the patient's body, for example out through the patient's mouth. Labeling, color-coding or other means may be used to help organize the suture tails. A device can be threaded onto the proximal ends of the sutures and parachuted into place. Optionally, the ends of the suture tails may have needles attached to facilitate passing the sutures through preformed holes or a sewing ring on the device.

FIGS. 15A and 15B show another embodiment of a delivery device 150 for deploying a T-tag fastener 100 mounted on the exterior of a flexible endoscope 142. FIG. 15A shows a penetrating cannula 114, which may be made of hypotube, 17-20 gauge, regular, thin or extra thin wall, 304 stainless steel or NiTi. The penetrating cannula 114 has a sharpened distal tip 116, which may be a short or standard hypodermic needle bevel, and a longitudinal slot 115, which is preferably 0.008-0.020 inch wide and around 0.5-1.5 inches in length.

A tubular member that functions as a garage or protective shield 152 for the penetrating cannula 114 may be mounted externally on the endoscope 142, e.g. with one or more interference fit mounting clips 144. The garage 152 has an ID larger than the penetrating cannula OD to allow sliding of the penetrating cannula 114 relative to the garage 152 with clearance and/or a slot 154 for the suture tail. The penetrating cannula 114 may be spring mounted in the garage 152, so that the penetrating cannula 114 retracts into garage 152 automatically or upon withdrawal from tissue.

The distal end of the transit cannula 156 connects to the proximal end of the garage 152. The garage 152 and the transit cannula 156 may be constructed of separate pieces of tubing as shown or, alternatively, they may be constructed of one continuous piece of tubing. The transit cannula 156 has a diameter the same or slightly larger than the penetrating cannula 114, optionally slotted, mounted externally on endoscope 142, e.g. with one or more interference fit mounting clips 144. In conjunction with the pusher 122, it delivers the T-tag fastener 100 to the penetrating cannula 114 while it is positioned in the garage 152. Preferably, it is designed to prevent binding when the endoscope 142 is deflected, including retroflexed, e.g. with a bellows or other flexible structure 158 at major flex points. The transit cannula 156 may be ferrous/magnetic for magnetic coupling between the transit cannula 156 and pusher 122. Alternatively, it could be a mechanical coupling or alternatively could use a monorail configuration.

A loading cannula or other fastener loading mechanism attaches to the scope biopsy port or scope handle. The loading cannula may load the pusher with T-tag fasteners individually or may feed T-tag fasteners from a magazine to pusher for delivery of multiple tags.

FIG. 16 is a detail drawing of a pusher 122 for use with a T-tag fastener delivery device 150. The pusher 122 sequentially advances the penetrating cannula 114, then deploys the T-tag fastener 100. The pusher 122 is preferably a stainless steel or NiTi wire, 0.008-0.025 inch diameter. Interface knobs 123 of diameter slightly smaller than the ID of transit cannula 156 are placed at intervals along the pusher 122 to allow free movement when the transit cannula 156 is flexed. Optionally, they can be magnets to assist coupling with the transit cannula. This embodiment shows a distal socket 121 to capture the T member of the T-tag fastener 100. In some embodiments a loose fit for easy release may be desired, while in others a press fit for retention and a secondary means to assist in release may be indicated. The distal socket 121 may have an optional slot for the suture tail. A spring loaded interface 125 engages the proximal end of the penetrating cannula 114 when the distal end of T-tag fastener 100 reaches the proximal end of the penetrating cannula bevel 116. The pusher 122 then advances the penetrating cannula 114 until it is fully extended and reaches a stop, the spring 124 then compresses and distal T member 102 is advanced out of penetrating cannula 114, pusher 122 is retracted leaving the T-tag fastener 100 attached to the tissue. The pusher 122 is then retracted and the penetrating cannula 114 then retracts. The pusher 122 may wind up onto a reel or drum attached to the loading cannula.

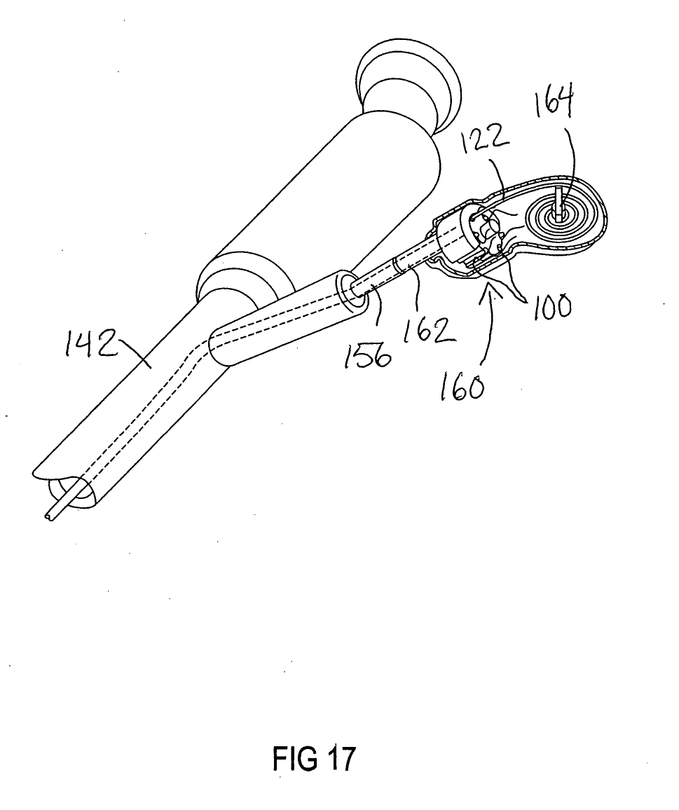

FIG. 17 shows a proximal end of a delivery device with a magazine 160 for sequentially delivering multiple T-tag fasteners 100. In this embodiment the transit cannula 156 passes through the biopsy channel of the endoscope 142. The proximal end of the transit cannula is near the biopsy port of the scope where a loading cannula 162 is attached with a rotating magazine 160 to feed T-tags 100 one at a time into the loading cannula 162. The loading cannula will have a slot to receive the T member and a coaxial narrower slot to allow passage of the suture tail of the T-tag. At the proximal end of the loading cannula 162 is shown a retractable pusher 122 configured to be coiled by a reel mechanism 164 to control pusher advancement and retraction. In this embodiment, the distal socket 121 on pusher 122 is retracted proximal to the magazine 160, the magazine is then rotated to position the next T-tag at the loading cannula 162 and then the pusher and attached socket are advanced.

The T-tag fastener delivery device could use any long tail T-tag fastener, including the dual headed T-tag fastener described herein and in the prior application.

Other aspects of T-tag fastener delivery devices 150 include:
1) ease of use related to exchange of devices;
2) management of the tails 104 of previously inserted T-tag fasteners 100.

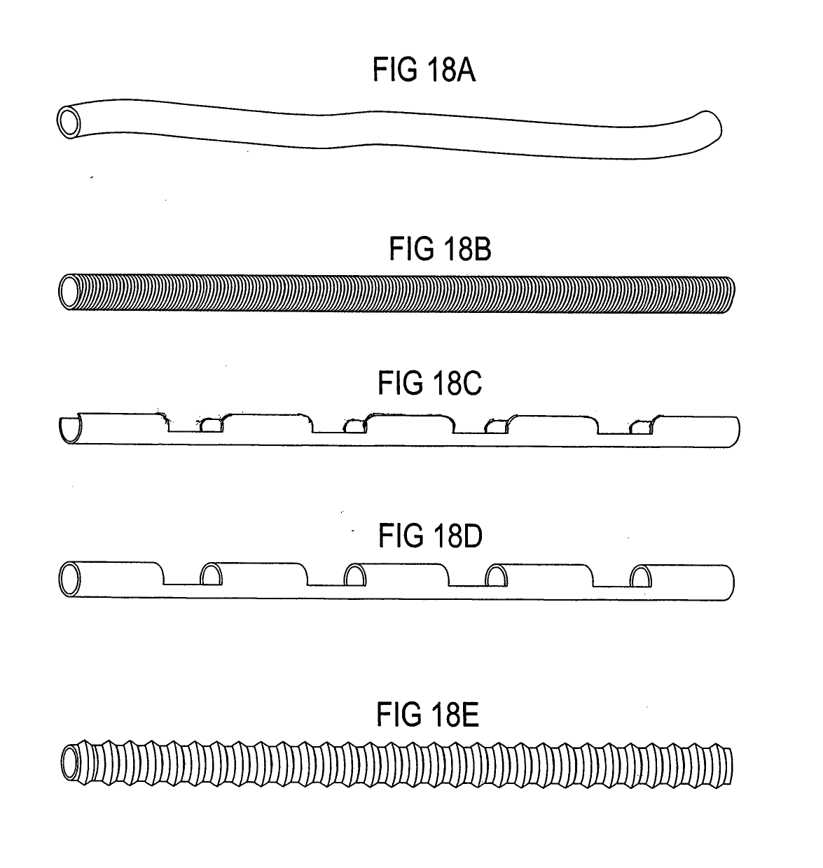

If used through an endoscope lumen or external to the endoscope, the delivery cannula or its components would be flexible to accommodate the flexing and articulations of the endoscope. Some examples of flexible constructions for the delivery cannula are shown in FIGS. 18A-18E. Flexibility can be provided, for example, by the following features singly or in combination, over the full length of the device/component or at selected locations:
1) flexible and/or elastic polymeric material (e.g. PU, PE, PEBAX) (FIG. 18A);
2) a superelastic metal material (e.g. NiTi);(FIG. 18A)
3) a coiled or braided material (e.g. 304 stainless steel with or without a polymer coating) (FIG. 18B);
4) a radially slotted material (e.g. 304 SS or NiTi) (FIGS. 18C and 18D);
5) a bellows (e.g. 304 SS or NiTi) (FIG. 18E).

Longitudinally slotted cannulas have advantages related to cannula sizing and also for delivering multiple T-tag fasteners. The suture tails of each T-tag fastener can exit the cannula through the slot after the fastener is deployed so that they will not damage or interfere with subsequently deployed fasteners. A parachute T-tag fastener or a snap T-tag fastener, described below, can have particular advantages in this regard. Any of the illustrated structures in FIGS. 18A-18E can be configured to include a longitudinal slot.

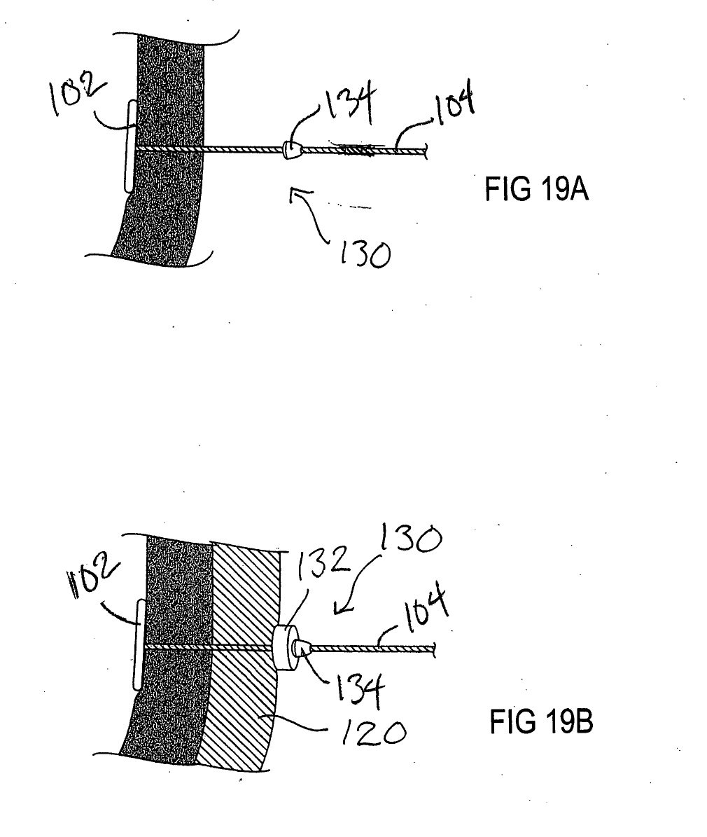

FIG. 19A shows a snap T-tag fastener 130 with a T member 102, a suture tail 104 attached to the T 102, and a snap member 134 attached to the suture tail 104. FIG. 19 B shows the snap T-tag fastener 130 of FIG. 19A with the cap 132 in place. Materials and dimensions of the snap 134 and cap 132 are optimized for ease of snapping (i.e. pushing cap 132 over snap 134) while maintaining sufficient retention force with the fastener 130. Snap force should be less than 1 kg and preferably less than 200 gm. Retention force should be greater than the snap force and preferably greater than 2.0 kg. The cap 132 is not threaded onto the suture tail 104 until after the suture tail 104 has been threaded through the device 120 to be parachuted. The features and properties of the snap T-tag fastener 130 can be combined with the features and properties of other T-tag fasteners described herein including, for example, use of an enlarged diameter stem as shown in FIGS. 2 and 3.

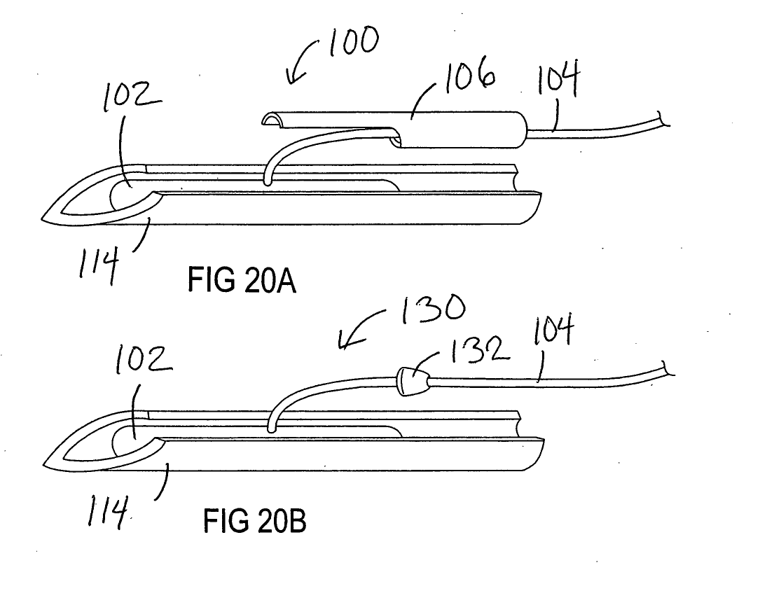

One aspect in common between the parachute T-tag fastener 100 and the snap T-tag fastener 130 is the presence of a relatively large element (i.e. the proximal T member 106 or the snap 132) on the suture tail 104 in proximity to the distal T member 102. With appropriate dimensioning, the proximal T member 106 of a parachute T-tag fastener 100 or the snap 132 of a snap T-tag fastener 130 remains outside the slotted penetrating cannula 114 and facilitates positioning the suture 104 outside of the cannula 114, as shown in FIGS. 20A-20B. Use of a delivery cannula system where all components (penetrating cannula, transit cannula, loading cannula) are similarly slotted allows multiple T-tag fasteners to be placed without interfering with the suture tails of previously placed fasteners. Also, a rail system as described herein can be combined with a slotted penetrating cannula to accomplish a similar result.

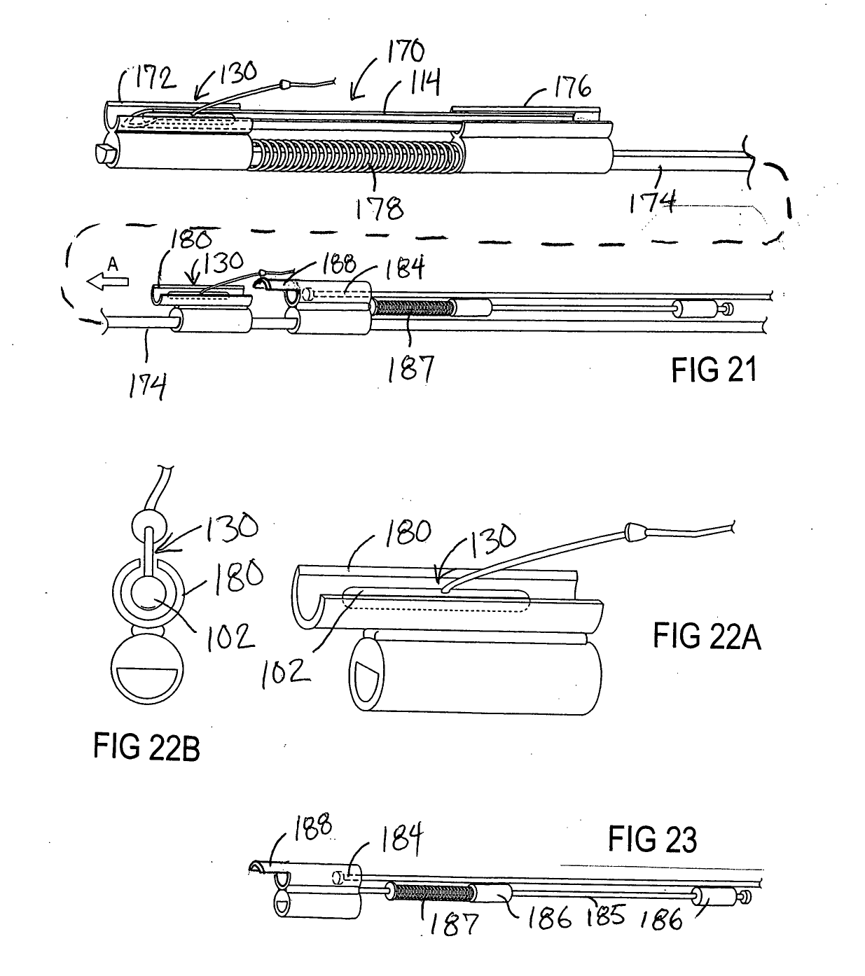

As a flexible endoscope bends in use, a precision longitudinal slot over the bending portion of the scope could be technically challenging. The fastener delivery apparatus 170 shown in FIGS. 21-23 could have advantages for T-tag fastener delivery compared to using an elongated longitudinally slotted cannula. FIG. 21 shows a slotted penetrating cannula 114 slidably received within a slotted garage element 172 mounted on a keyed wire 174. A snap T-tag fastener 130 is shown loaded into the penetrating cannula 114. The proximal end of the penetrating cannula 114 is attached to a slotted socket 176 that is slidably mounted on the keyed wire 174. A compression spring 178 is positioned between the garage element 172 and the socket 176. The penetrating cannula 114 and socket 176 move together, compressing the spring 178 to advance the penetrating cannula 114. The distance separating the garage 172 from the socket 176 is limited by the collapsing spring 178. The keyed wire 174 attached to the garage 172 extends the full length of the scope back to the loading cannula. The loading cannula can be in the form of a cartridge that can be loaded on a pusher assembly outside the scope.

FIG. 22A shows a slotted loading cannula or cartridge 180 with a second snap T-tag fastener 130 positioned for loading into the penetrating cannula 114. FIG. 22B shows a distal end view of the slotted loading cannula or cartridge 180. The distal end of the slotted loading (or transit) cannula 180 mates with proximal end of the slotted socket 176 attached to the penetrating cannula 114. The loading cannula 180 can be used or as preloaded cartridges to facilitate handling of T-tag fasteners 130. Multiple cartridges can be loaded on a magazine, which can feed them into the loading cannula 180, which is slidably mounted on the keyed wire 174 proximal of the penetrating cannula 114 and the garage 172. If the loading cannula assembly is used as a cartridge, they will be fed onto the proximal end of the keyed wire 174.

FIG. 23 shows the pusher assembly 182. The pusher assembly 182 includes a socket 188 that is slidably mounted on the keyed wire 174 and a pusher rod 184 that slidably mounted within the socket 188. A compression spring 187 is mounted between a boss 186 on the pusher rod 184 and the socket 188 so that it resists advancement of the pusher rod 184 with respect to the socket 188. The pusher assembly 182 is positioned on the keyed wire 174 just proximal to the loading cannula 180, as shown in FIG. 21.

When the pusher assembly 182 is advanced distally on the keyed wire 174, the socket 188 engages and then pushes the loading cannula 180 distally until it engages the slotted socket 176 and the penetrating cannula 114. The pusher rod 184 first advances the snap T-tag fastener 130 from the loading cannula 180 into the penetrating cannula 114. As the pusher rod 184 moves farther distally, the penetrating cannula 114 advances distally out of the garage 172 to pierce the tissue. Once the penetrating cannula 114 is fully extended, the pusher rod 184 deploys the T-tag fastener 130 by pushing it distally out of the penetrating cannula 114. Furthermore, the relative spring rates of the penetrating cannula spring 178 and pusher assembly spring 187 are such that the penetrating cannula 114 is fully deployed before the pusher assembly spring 187 begins to collapse. Use of a spring in this manner prevents inadvertent or premature T-tag fastener deployment. Pusher assembly spring travel is of sufficient length to fully expel the T member from the penetrating cannula 114. For example, if approximately 250 gm of penetration force is desired to be transmitted to the penetrating cannula, the penetrating cannula spring may be fully collapsed at 250 gm and the pusher assembly spring will begin to collapse at 250 gm and may be fully collapsed at 275 to 300 gm.

The interface between the proximal end of the keyed wire 174, the T-tag fastener cartridge magazine and the pusher assembly 182 can all be combined in a deployment handle assembly similar to the one shown in FIG. 17, which could optionally deploy both the keyed wire and the pusher wire assembly, or the pusher wire assembly alone, from one or more reels. The deployment handle could be secured to the proximal end of an endoscope lumen or external to the endoscope control handle if the garage assembly is secured externally to the scope on its distal end. If through the endoscope lumen, the keyed wire would then be extended through the scope (optionally, before being placed into the patient.) The magazine could then be attached to the handle and a single cartridge advanced into a loading area where the pusher wire would be engage, then the cartridge and pusher wire, mated together, could be advanced onto the proximal end of the keyed wire.

In this embodiment, once the loading cannula and cartridge are mated, further advance of the mated cartridge and pusher rod would result in:
1) transit of the T-tag fastener in its cartridge to the distal end of the endoscope;
2) penetrating cannula extension;
3) holding and/or advancing the penetrating cannula when tissue is pierced (could be simultaneous with step (1)
4) holding penetrating cannula extended when T-tag fastener is deployed.

Retraction of the pusher rod would:
1) allow retraction of the penetrating cannula through the tissue;
2) pull the cartridge in a proximal direction to the proximal end of the endoscope (an interface e.g. mechanical or magnetic would be required);
3) position the cartridge for being expelled into a storage chamber.

When placing T-tag fasteners it can be beneficial to orient the T members in a specific direction relative to the anatomy. Two approaches are discussed:
1) orientation by delivery cannula;
2) orientation by pusher.

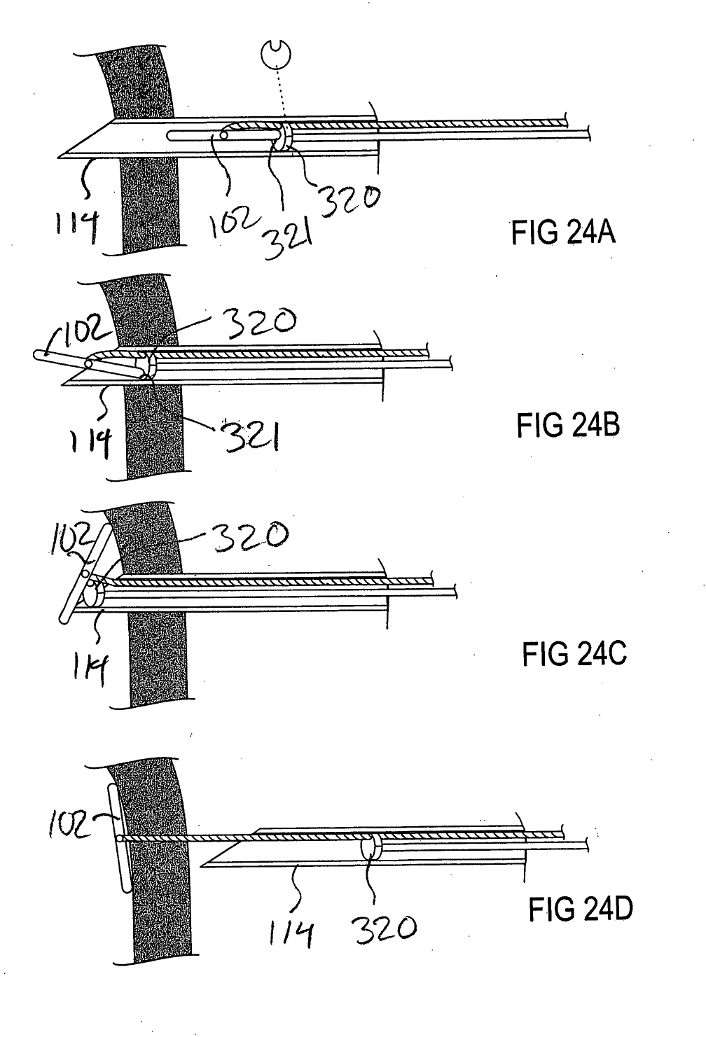

If a non-slotted penetrating cannula is used, then an oriented pusher or keyed cannula or keyed pusher and keyed T member can be used to control the orientation of the T member as it exits the delivery cannula. FIGS. 24A-24D show an example of a method of delivery orientation control that involves a keyed pusher 320 with a mating keyed portion 321 on the proximal end of the T member 102. In this case the pusher key 320 can also have a keyed delivery cannula 114 so the directional orientation is based upon the delivery cannula orientation. If the pusher 320 is not keyed to the delivery cannula 114, rotation of the pusher 320 may be sufficient to determine the directional orientation of the T member delivery. A lubricious coating on the pusher shaft may facilitate rotational control.

Use of a slotted delivery cannula with the suture tail of the T-tag fastener positioned through the slot can maintain orientation of the T member as it passes through the delivery cannula. This may be sufficient to orient the T member, but the suture tail will generally exit the slot before the T member is fully deployed. Keying the proximal portion of the T member to the slot can improved control of orientation during deployment.

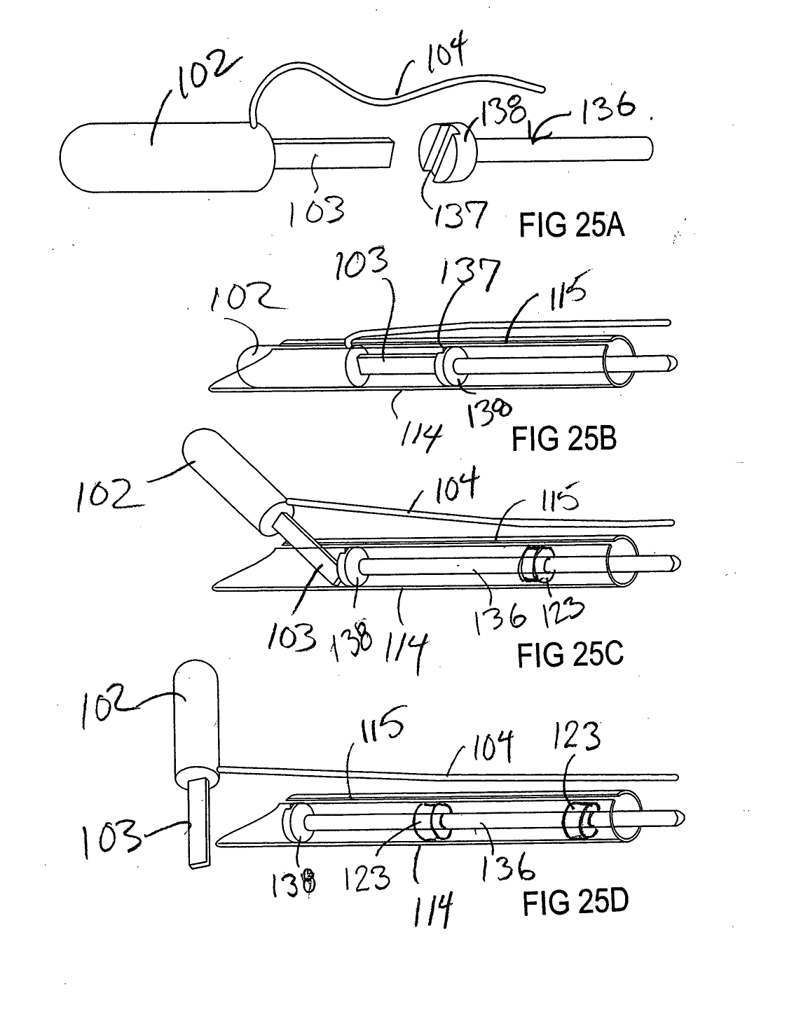

As shown in FIGS. 25A-25D, this can be accomplished by using a flattened portion 103 extending from the proximal end of the T member 102 that can serve as a key that engages the slot 115 and maintains the orientation of the T member 102 as it rotates during deployment. To deploy this T-tag fastener, it is helpful to apply tension to the suture tail 104 while slowly advancing the pusher 136 to cause the T member to rotate and lay against the tissue before the pusher 136 ejects the flat portion 103 from the delivery cannula. To help orient the T member 102, the pusher 136 can be configured with an interface knob head 138 having a slot 137 that engages the flat portion 103 of the T member 102 and holds it aligned with the slot 115 in the penetrating cannula 114.

Another aspect of the T-tag fastener delivery device shown in FIGS. 25A-25B is that is shows another means to force or maintain the suture tail 104 external to the slotted penetrating cannula 114. The pusher assembly is configured to exclude the suture tail 104 of the T-tag fastener from the slot 115 of the slotted penetrating cannula 114 by the sizing of interface knob head 138 and, optionally, one or more other interface knobs 123 positioned along the pusher rod 136.

The T-tag fastener delivery device may have a fixed orientation with respect to the endoscope. Preferably, a visual indication of the delivery device orientation is provided for the endoscope operator. Alternatively, the delivery device may be rotatable with respect to the endoscope. This will require a mechanism for controlled rotation of the delivery device, e.g. a rotating shaft with gears to transmit the rotation to the delivery cannula, and a visual indication that changes to indicate the rotational orientation of the delivery cannula. With a video endoscope, this can be done electronically and indicated on the viewing monitor.

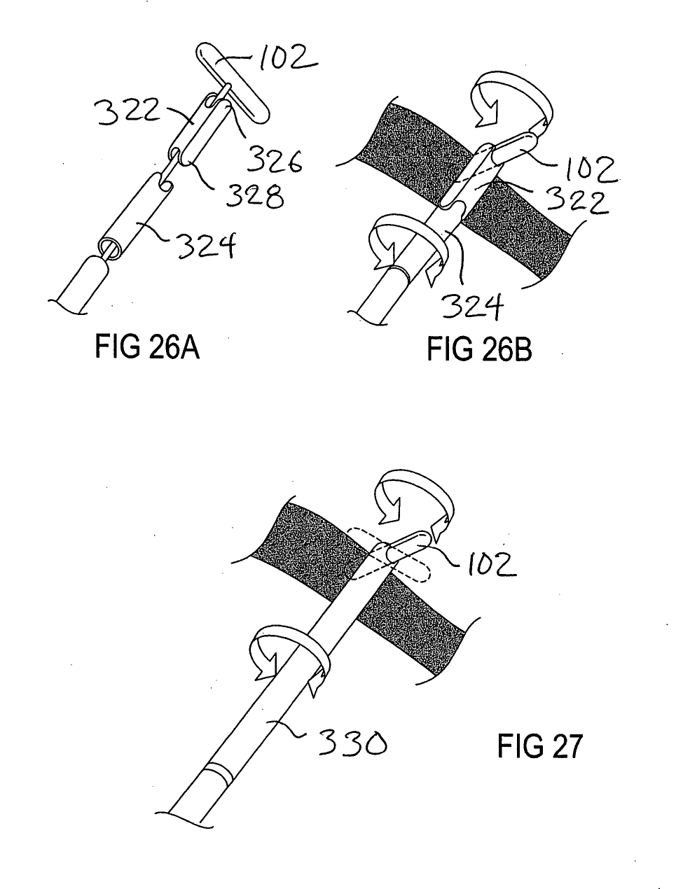

It is also possible to orient a T member 102 of a fastener after insertion. As shown in FIG. 26A, an attachment spacer or standoff 322 that is part of the fastener could be configured to transmit rotation from the delivery device or another tool 324 to the T member 102. Coupling structures 326, 328 can be incorporated on the distal and proximal ends of the standoff 322. The coupling structure 326 at the distal end of the standoff 322 would be configured to engage the deployed T member 102 and the coupling structure at the proximal end 328 of the standoff would be configured to mate with the delivery device 324, which will have a rotational drive mechanism. The standoff 322 would transmit rotation from the drive mechanism to the T member 102 as shown in FIG. 26B and after the delivery device 324 is removed the standoff 322 will serve as a spacer between the T member 102 and the attached device.

Similarly, the T member can be oriented directly by a rotational drive tool 330 that is passed over the suture tails and through the tissue, where it engages the side of the T member 102 to transmit rotation to the deployed T member 102 as shown in FIG. 27. The tool 330 is removed once the T member 102 has been rotated to the desired orientation.

Rollers, a low friction coating or other material or structure on the T member of the fastener would allow the fastener to slide on the serosa and thereby direct itself to a desired orientation. A roller or low friction material on the T member would also facilitate reorienting the T member to a desired orientation using a rotational drive tool as described above.

One other aspect of T-tag fastener delivery is the potential for a T member to pass proximally through the track formed by the penetrating cannula rather than rotating to be parallel to the tissue surface as desired. In many cases the connection of the T member and the stem, for example as shown in FIG. 2, can induce rotation of the T member. If the T member is attached to a suture tail, for example as shown in FIGS. 3 and 20A, other means may be desirable to induce T member rotation. The structures shown in FIGS. 24A-24D and 25A-25D can be used, with or without orientation control, to induce T member rotation and avoid inadvertent retraction of the T member through the tissue track.

One of the challenges when performing endoscopic suturing, stapling or other types of attachment, e.g. with T-tag fasteners, is that the tissue tends to move away from the attachment device. A separate grasper can be inserted through an instrument lumen in the endoscope for holding the tissue, but this approach has its drawbacks because it is very difficult to achieve a good cooperation between the two instruments. To facilitate endoscopic attachment methods, a better cooperation can be achieved when an attachment device is combined with or otherwise mechanically linked to a grasper.

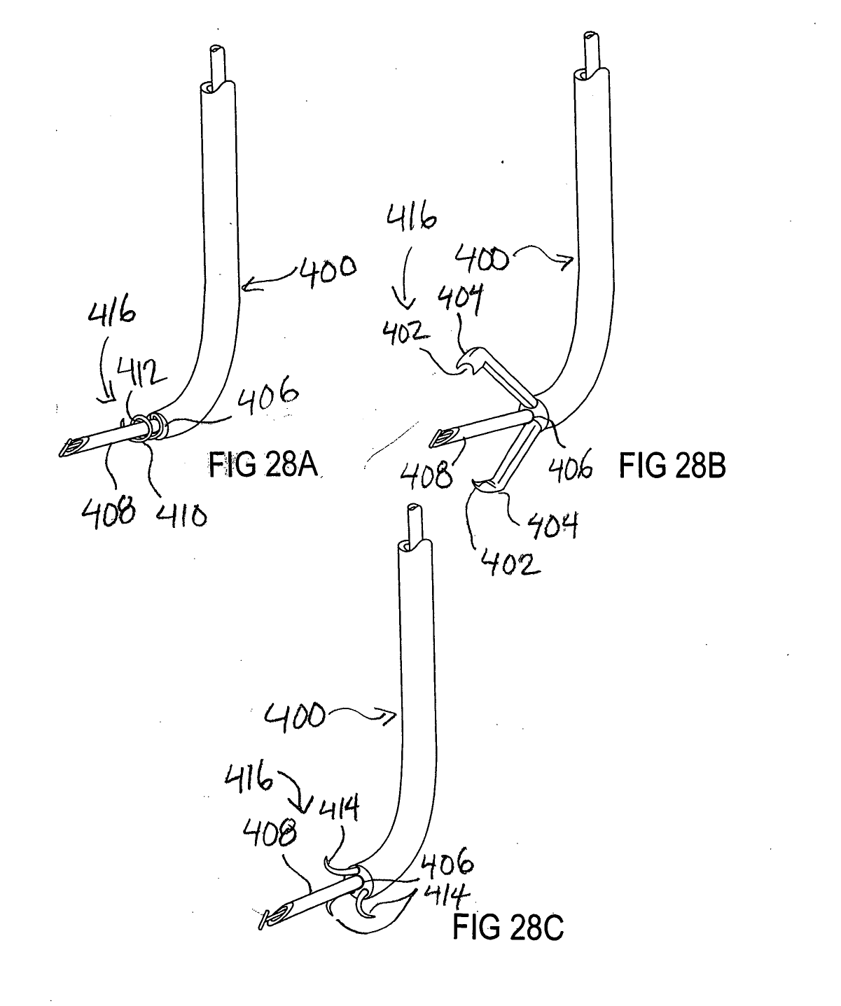

In one embodiment shown in FIG. 28A, the combined instrument 400 includes a flexible grasping forceps 416, for example a rat-tooth grasper, with an opening 402 through the jaws 404 of the grasper. Coaxial with the jaws 404 of the grasper 416 is a lumen 406 for passing an attachment device 408 (shown extended) through the opening 402 in the jaws 404. The grasper operating mechanism must be modified to accommodate the coaxial instrument lumen 406. The attachment device 408 may be an endoscopic suturing device, a stapler, a T-tag fastener delivery device or other known endoscopic attachment device. Alternatively, the lumen 406 for passing the attachment device may exit the shaft beside the jaws 404 of the grasper, potentially simplifying the construction of the grasper operating mechanism and obviating the need for an opening in the jaws. In another alternative configuration, the attachment device may be integrated into the combined instrument.

In another embodiment shown in FIG. 28B, the combined instrument 400 includes a corkscrew-type grasper 410 with a lumen 406 for passing an attachment device 408 (shown extended) coaxial with the opening 412 through the center of the corkscrew grasper 410.

In another embodiment shown in FIG. 28C, the combined instrument 400 includes a grasper 416 with multiple curved needles 414 that penetrate the tissue in opposing directions to grasp the tissue. Coaxial with the grasper 416 is a lumen 406 for passing an attachment device 408 (shown extended) between the curved grasping needles 414.

The combined instrument 400 is introduced endoscopically and the distal end is maneuvered into contact with the tissue to be attached. The grasper 416 is actuated to hold the tissue and the attachment device 408 is passed through the lumen to deliver a suture needle or fastener into or through the tissue. Because the grasper 416 and the attachment device 408 are so closely linked, the tissue cannot move out of the way of the attachment device 408, allowing the suture or fastener to be delivered through the tissue reliably and efficiently.

Alternatively or in addition, a vacuum coupling cuff on the distal tip of the endoscope can be used to allow vacuum holding of the tissue during attachment.

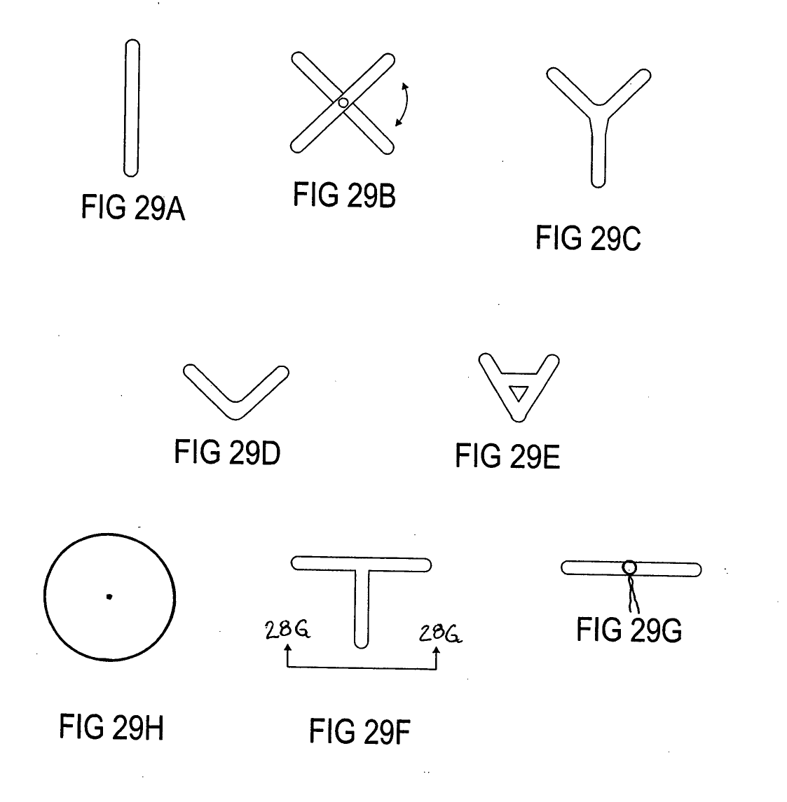

In some applications of the T-tag fastener, it will be advantageous to provide different configurations for the T member(s). FIGS. 29A-29H show top views of some possible T member configurations. FIG. 29A shows a straight bar-shaped T member. FIG. 29B shows an X-shaped T member. FIG. 29C shows a Y-shaped T member. FIG. 29D shows a V-shaped T member. FIG. 29E shows an A-shaped T member. FIG. 29F shows a T-shaped T member. FIG. 29H shows a circular or disc-shaped T member. The X, Y, V, A and T-shaped members may optionally be elastically deformable or pivotable at the center to provide a low insertion profile. FIG. 29B shows an example of such a pivot. These configurations of T member will provide greater anchoring force and/or reduced pressure on the tissues where they are attached. Although these T members have different configurations in the top view, they will still have an approximately T-shaped configuration in the side view, as shown in FIG. 29G, which is a side view of the T-shaped T member in FIG. 29F.

Expandable or swellable T members also have advantages for greater anchoring force and/or reduced pressure on the tissues where they are attached. A T member could be configured of a material that is initially small and/or soft for insertion of the T members. After insertion, the T member would expand or swell and then harden in the expanded configuration. This could result from a chemical reaction that is initiated by absorption of water or another reactant. A reagent in the material of the T member or added to it after insertion could initiate or catalyze the reaction of a hardenable material, e.g. a cyanoacrylate adhesive. Various materials and configurations for this function are described in the parent application, Ser. No. 10/698,148.

Other enhancements can be applied to the T-tag fasteners described herein and those described in the parent application, Ser. No. 10/698,148 and provisional 60/613,917. For example, the T member of the T-tag fastener can be configured to minimize pressure concentrations on the tissue. When applied by direct insertion, the T member of the T-tag fastener can be configured to distribute forces over a larger surface area. For example, the T member can be configured as a disk, square, rectangle or other shape with a large surface area. For blind insertion, the T member of the T-tag fastener can be configured to expand after insertion through the tissue to distribute forces over a larger area. For example, the T member can expand to form a disk, square, rectangle, I, X, Y or other configuration with a large surface area, as described herein (e.g. FIGS. 29A-29H) and in the prior application. Alternatively or in addition, to reduce the potential for erosion at the end of the T in some clinical situations it could be beneficial for the ends of the T to have increased dimensions or configurations (for example a round ball shape) to reduce pressure at the end of the T and/or increased flexibility which will result in a reduction of the angle between the gastric wall and the ends of the T. This would reduce the forces between the T and the gastric wall and therefore reduce the potential for erosion at the ends. Structures that could accomplish this could include tapered thickness or cross section to reduce the bending moment. Alternatively or in addition, changes in material properties such as hardness, bending modulus and/or elongation can accomplish the same result. For example the T near the stem could be of a material of a durometer such as Shore 65D or higher the material may change as one moves out along the arms of the T transitioning through 55D/100A to 90A durometer or lower. Rounding, smoothing and structures that otherwise distribute forces over a larger area will also serve to reduce erosion at the ends of the T. A circular shaped T may be particularly desirable to reduce erosion.

All or a portion of the fastener can be coated and/or made with a material that will encourage tissue ingrowth to create a seal and to promote a strong and durable attachment. All or a portion of the fastener can be coated and/or made with a swellable material to create a seal and/or to spread out the force of attachment over a greater surface area, thereby reducing the pressure on the tissue. All or a portion of the fastener can be coated and/or made with a material that is biodegradable or bioresorbable. Examples of such coatings materials are described in the parent application, Ser. No. 10/698,148.

In some of the examples herein and in the prior application, the T-tag fasteners are placed transmurally or through a full thickness plication. In an alternate method, an intramural T-tag can be placed submucosally, preferably in the muscularis, where the T member would anchor the suture. The T would have a structure that is all or partly biodegradable. In this way, after the initial weakening of the tissue that occurs soon after the initial suturing, all or part of the T would degrade leaving the suture (with or without some supporting structure) securely anchored in the tissue by fibrotic tissue that would form around the degrading T.

Following are descriptions of attachment devices and other means for securing an implantable device within the gastrointestinal system. The implantable devices and/or attachment means can be configured to avoid causing excessive pressure within the tissue by having compliance that is compatible with the gastrointestinal tissues where it is attached. Device compliance can also be important for providing a leak free seal between an implanted device and the tissue at the attachment point. Compliance can be provided in the radial or circumferential direction and/or in the vertical, axial or longitudinal direction. The device may have different compliance in different regions to be compatible with the tissue at the attachment point and at other portions of the gastrointestinal tract through which it runs. The device may have different compliance in different directions to be compatible with the tissue at the attachment point while simultaneously achieving other goals of the device. Compliance can be provided in a number of different ways. One way is by elastic or plastic deformation of the device and/or the attachment means. Another way is by a mechanical decoupling that allows relative movement between the device and the attachment points, and/or between the attachment points themselves, without transmitting excessive force or pressure to the tissue.

In some clinical situations, it will be desirable to match compliance between the device and the tissue to which it is attached. In other situations, based upon the clinical situations, it will be desirable to provide a device with higher or lower compliance than the tissue to achieve certain objectives. For example, maintaining the position of the proximal end of an attached sleeve device will require a device that is relatively noncompliant in at least the axial direction.

The implantable devices and/or attachment means described herein can utilize one or more of the following features to modify the compliance:
1) Highly-elastic materials (large amounts of stretch with low forces)
   a) Composite structures with elastic or super-elastic portions
2) Pleated materials (minimal force until pleats straighten)
   a) Similarly, other types of loose (gathered) or hanging (e.g. dangling sutures) connections
3) Fenestrated structures (e.g. cuts or slits)
   a) Can optionally use slidable overlapping elements to reduce/eliminate leaks at the slits
4) Stretchable weaves or knits
   a) Cylindrical and/or flat (e.g. an expandable or self-expanding stent or fabric)
5) Elastic, hinged and/or slotted structures that allow relative motion of components
   a) Can also use overlapping for leak control
6) Isolated or independent attachments
   a) Attachments to the GI tissue that are not connected
      i) They can initially be isolated and later connected
      ii) They could be interfaced with another device in a manner that does not restrict their relative motion (e.g. long hanging tethers)
7) Combinations of the above

Other features may be incorporated in such structures such as:
1) Reinforcement at attachment points
   a) e.g. incorporation of fabric in a molded elastic structure
2) Clips, hangers or other means for sleeve interface at individual points
   a) For both isolated coupled and decoupled interfaces
3) Materials that encourage ingrowth and/or overgrowth
4) Separation of the functions of attachment and sealing
   a) To allow greater compliance at the attachment without increasing leakage
5) Means to maintain a substantially constant restricted volume within and between the device and stomach.
6) Through control of the degree and direction of device compliance.

Certain methods for the use of such structures include:
1) Treating the tissue at the GEJ to eliminate or minimize distention
   a) Creation of fibrotic or scar tissue
      i) Chemical, RF or other energy
      ii) Permanent or temporary to allow healing/seasoning of the attachment
   b) Thereby reducing the requirement for a compliant cuff or other attachment
2) Treating the tissue at the GEJ to increase tissue strength
   a) Creation of fibrotic or scar tissue
3) Allowing time for a primary attachment to heal before implantation and attachment of one or more secondary devices e.g. a sleeve.

Another strategy for avoiding excessive pressure on the gastric and esophageal walls and the complications of ischemia and pressure necrosis is to spread out (over a substantial surface area) and/or otherwise reduce the attachment force. FIGS. 30A-30B illustrate an attachment ring 260 for a gastrointestinal sleeve device 258 that uses these principles. The attachment ring 260 has a generally flat annular ring 262 with a central opening 264 sized for attachment of a gastrointestinal sleeve device 258. The ring 262 may be molded of a biocompatible polymer or it may be constructed of Dacron or another fabric coated or impregnated with a biocompatible polymer, such as silicone or polyurethane. The attachment ring 260 has a relatively large diameter Dacron cuff 266 around the outer periphery to spread out the attachment force over a substantial surface area of the tissue. The stomach wall is folded into a double plication around the Dacron cuff 266 and fastened with sutures, rivets, T-tag fasteners 268, or the like. The thickness of the Dacron cuff 266 also avoids sharp bends at the tissue plication, which will help to reduce pressure on the tissue. Preformed attachment holes 270 may be provided in the attachment ring 260 to facilitate deployment of the fasteners 268 and to provide some strain relief at the attachment points. Ingrowth of tissue into the Dacron cuff 266 over time will help to increase the strength of the attachment to the gastric wall.

Preferably, the attachment ring 260 is compliant in the radial direction so that expansion and contraction of the stomach and esophagus due to contents and/or muscular action will not place additional, or actually reduce, stress on the attachment points. An elastomeric material, such as silicone or polyurethane that provides approximately 150% or more stretch in the radial direction is preferred. At the same time, the attachment ring can have enough lateral rigidity to act as a mounting platform for the gastrointestinal sleeve device and to resist downward movement due to the weight of the gastrointestinal sleeve device and its contents and peristaltic traction on the sleeve. The lateral rigidity of the attachment ring can be enhanced with radially oriented bending reinforcements, such as ribs or embedded reinforcement members 272. Alternatively, the attachment ring can be flexible and compliant and other means such as hooks, sutures staples, etc., can be used for sleeve attachment.

Another strategy for avoiding excessive pressure on the gastric wall at the attachment points is to reduce the weight that the device attachment must support. This can be accomplished with spiral or longitudinal reinforcement members and/or inflatable balloons for structural support, particularly in the gastric portion of the gastrointestinal sleeve device, as described in the prior application. These features will help to transfer some of the weight to other structures of the stomach such as the antrum or the pylorus and will reduce the tension on the attachment at the GEJ. Likewise, additional attachments points at other points in the stomach will help to reduce the tension on the attachment at the GEJ. Attachment at the pylorus or other points in the stomach will also provide an added measure of safety. If the primary attachment at the proximal end of a sleeve device ever came unfastened, these additional attachment points would prevent the sleeve device from passing through the pylorus and becoming lodged in the intestine.

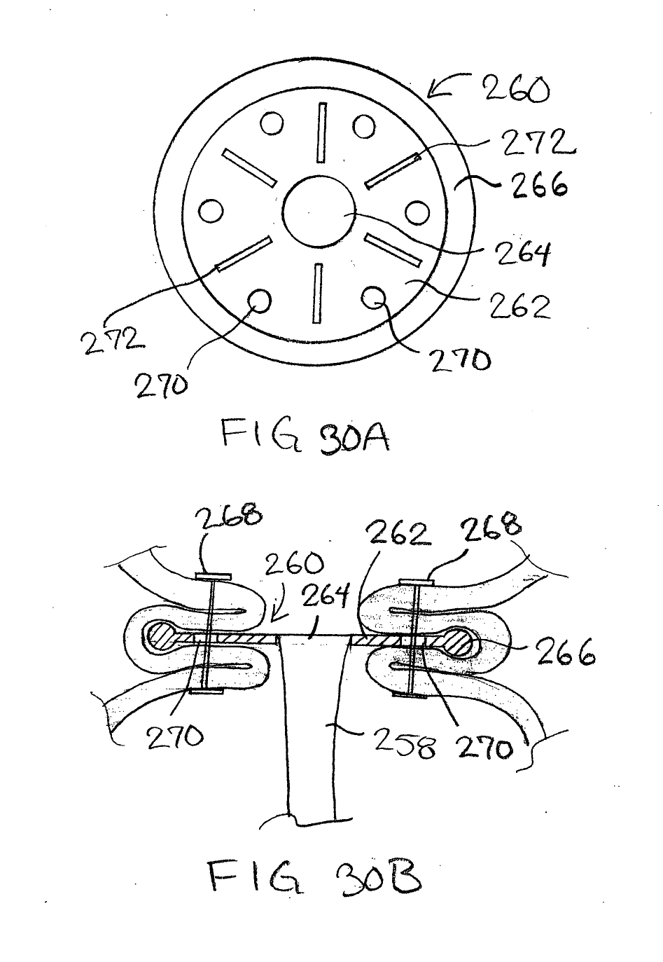

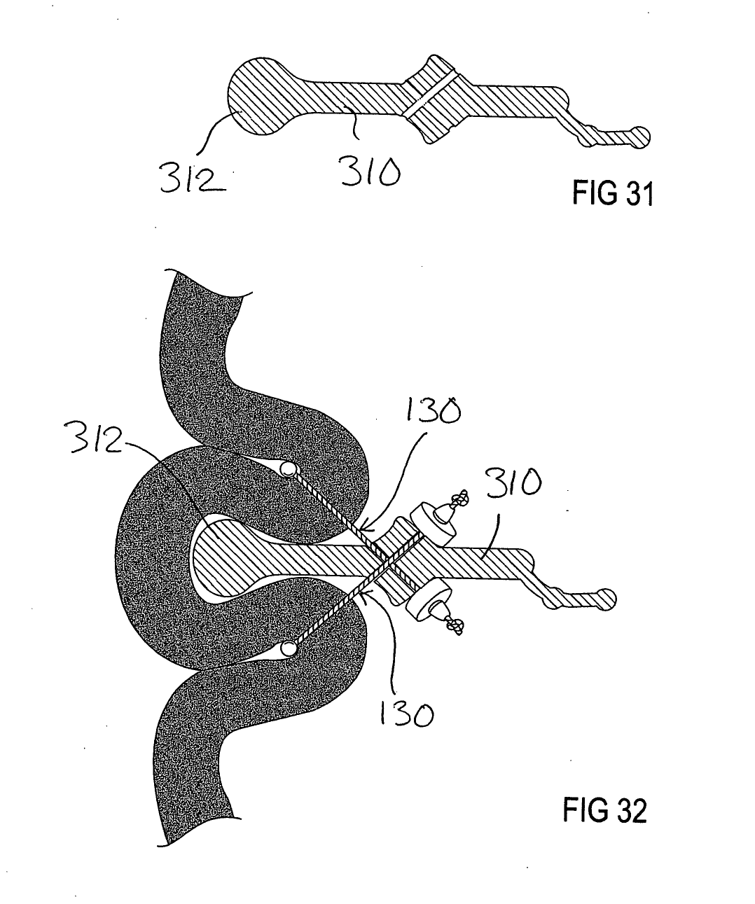

FIGS. 31 and 32 show a cross section of one half of an attachment ring device 310 that uses a double plication wrapped around a large ring 312, which prevents lateral motion of the stomach wall. The large ring 312 distributes the force on the gastric wall. Two rows of T-tag fasteners 130 or other types of fasteners attach the device to the double plication. Various means can be used to secure the T-tag fastener tails. Tissue contact portions of the attachment ring 310 can include ingrowth enhancing means as described herein. Ingrowth and attachment of the T-tag fasteners 130 through the attachment ring prevents any cheese-cutter effect of the T-tag fastener tails. This use of T-Tag attachment can be adapted to the compliant attachment ring shown in FIGS. 30A and 30B.

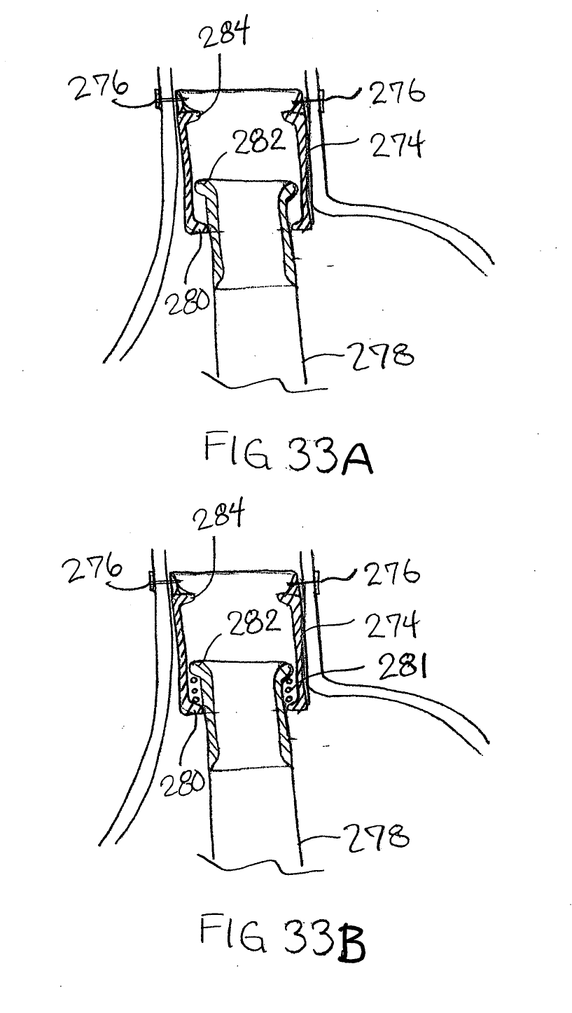

Another strategy for avoiding excessive pressure on the gastric wall at the attachment points is to provide an axially “floating” attachment for the gastrointestinal sleeve device so that stress transferred to the esophageal or gastric walls can be minimized or controlled. For example, FIG. 33A illustrates another means for attaching a gastrointestinal sleeve device that uses a compliant expandable or self-expanding stent 274 within the patient's esophagus. The gentle expansion of the stent 274 anchors it in the esophagus without placing undue pressure on the tissues. One or more T-tag fasteners 276 or other fasteners may be used as an additional attachment means. Rather than being solidly attached to the stent 274, the proximal end of the gastrointestinal sleeve device 278 has a floating attachment so that that expansion and contraction of the stomach and esophagus due to contents and/or muscular action will not place additional stress on the attachment points. One example of a floating attachment is to have an annular ridge 280 on the inside of the stent 274 and a ring 282 on the proximal end of the gastrointestinal sleeve device 278. The ring 282 is sized so that it merely rests on top of the annular ridge 280, but cannot be pulled through it. The annular ridge 280 supports the gastrointestinal sleeve device, but it does not transfer any radial force from the gastrointestinal sleeve device to the esophageal or gastric walls. Optionally, a metal coil or other type of spring 281 can be used to couple the ring 282 and annular ridge 280, as shown in FIG. 33B. If desired, the floating attachment can also be combined with other features to allow the gastrointestinal sleeve attachment to float or expand and contract in the radial direction, as well as in the longitudinal direction. Optionally, a proximal annular ridge 284 may be formed near the proximal end of the expandable stent 274 to limit longitudinal motion of the floating attachment in the proximal direction. Other means, such as the fasteners described herein and those described in the prior patent application can be used in place of the stent to fasten a floating attachment of this type at the GEJ.

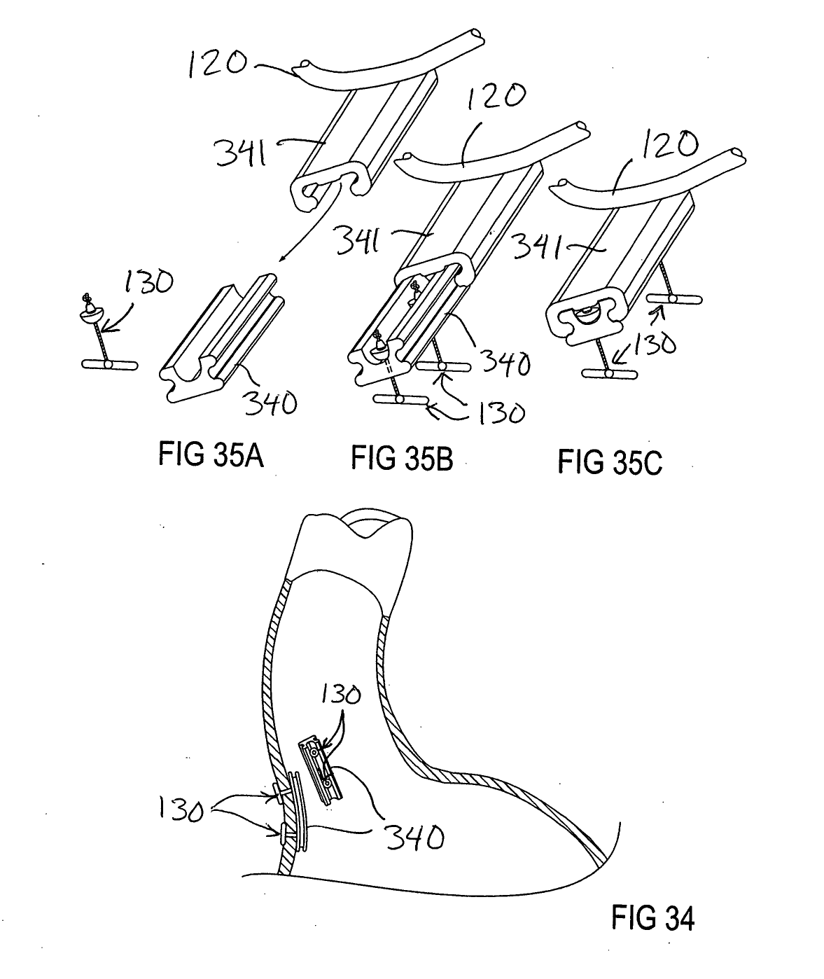

An alternate means of implementing an axial floating attachment uses vertically mounted isolated sliding attachment members can be used as an attachment structure for an implanted device. FIGS. 34 and 35A-35C show an example of vertically mounted isolated sliding attachment members 340 in a patient's stomach. This allows a maximum of relative motion between attachments with a minimum of force resisting that motion. The sliding vertical attachment allows vertical motion similar to that achieved with the floating attachment shown in FIGS. 33A-33B. Each of the isolated attachment members 340 is attached to the wall with one or more T-tag fasteners 130 or other types of fasteners. Typically, 4-16 attachment members will be fastened around the periphery of the esophagus or stomach in the vicinity of the GEJ. The length of the attachment members helps to distribute the attachment force or pressure exerted on the tissue. The attachment members could be completely separate or they may linked to one another by high compliance members or a membrane that would help to position and orient the members for attachment, but that would allow the members to float like separate attachment points. The linking members or membrane may be configured to encourage ingrowth/overgrowth for attachment and sealing. Alternatively, the device that is mounted on the attachment members may provide a seal against the gastric mucosa.

Once the attachment members 340 have been fastened to the stomach wall, the implantable device 120 is connected to them using a like number of sliding connectors 341 attached to the implantable device 120. The sliding connectors 341 are configured allow vertical movement of the implantable device 120 with respect to the attachment members 340 and the stomach wall. Stops or detents may be included to limit the vertical movement of the implantable device 120. In the example shown, the sliding connectors 341 are configured as channels that are slidably connected to rail-shaped attachment members 340. Other configurations of attachment members 340 and sliding connectors 341 are also possible.

The use of isolated attachments to attach a sleeve within the GI tract has been previously disclosed herein and in parent application, Ser. No. 10/698,148. Isolated attachment allows a maximum of relative motion between attachments with a minimum of force resisting that motion. As has been discussed, the attachments can be left in place for a time to heal and become secure prior to the attachment of a sleeve. This concept can be extended to the use of a cuff, which interfaces a replaceable sleeve with GI tissue. Isolated attachments can be placed in the GI tissue, a period of time can allow healing of these attachment points and then the reusable cuff can be fastened to the GI tissue using the previously placed isolated attachments.

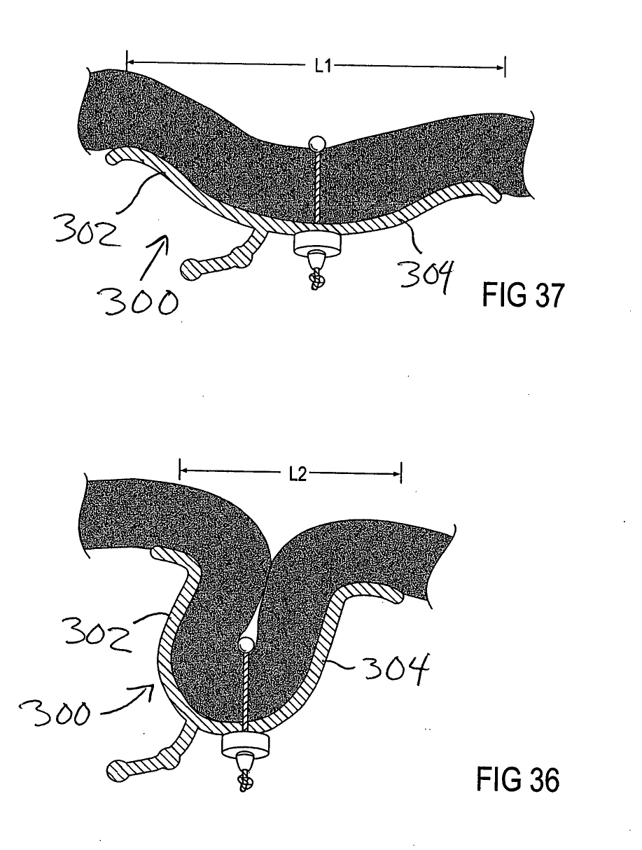

FIGS. 36 and 37 show one manner by which larger gastric wall motion can be accommodated by forming the device/gastric wall interface in a compliant manner. This can be a compliant device or a means by which the device can move to minimize motion relative to the tissue. Specifically, as tissue moves, the device would move with the tissue rather than resist the motion, which could lead to mucosa/device separation. However, device motion could be limited to the ability of the mucosa to maintain its integrity.

FIG. 36 shows a compliant attachment ring device 300 for use with T-tag fasteners or other types of fasteners. The attachment ring device 300 creates a plication (fold) and then controls the force to maintain the plication against the forces in the plicated tissue that would tend to straighten the fold. In FIG. 36 the attachment ring device 300 is shown in a normal resting position, flexible or compliant upper 302 and lower 304 flanges on the ring help maintain the shape of the plication. FIG. 37 shows the compliant attachment ring device 300 with tension exerted on the tissue at the tissue/device interface. The flexible or compliant upper 302 and lower 304 flanges on the ring open up to compensate for the tension, which reduces the force seen at the attachment points. When the tension is reduced, the compliant attachment ring device 300 will return to its normal resting position.

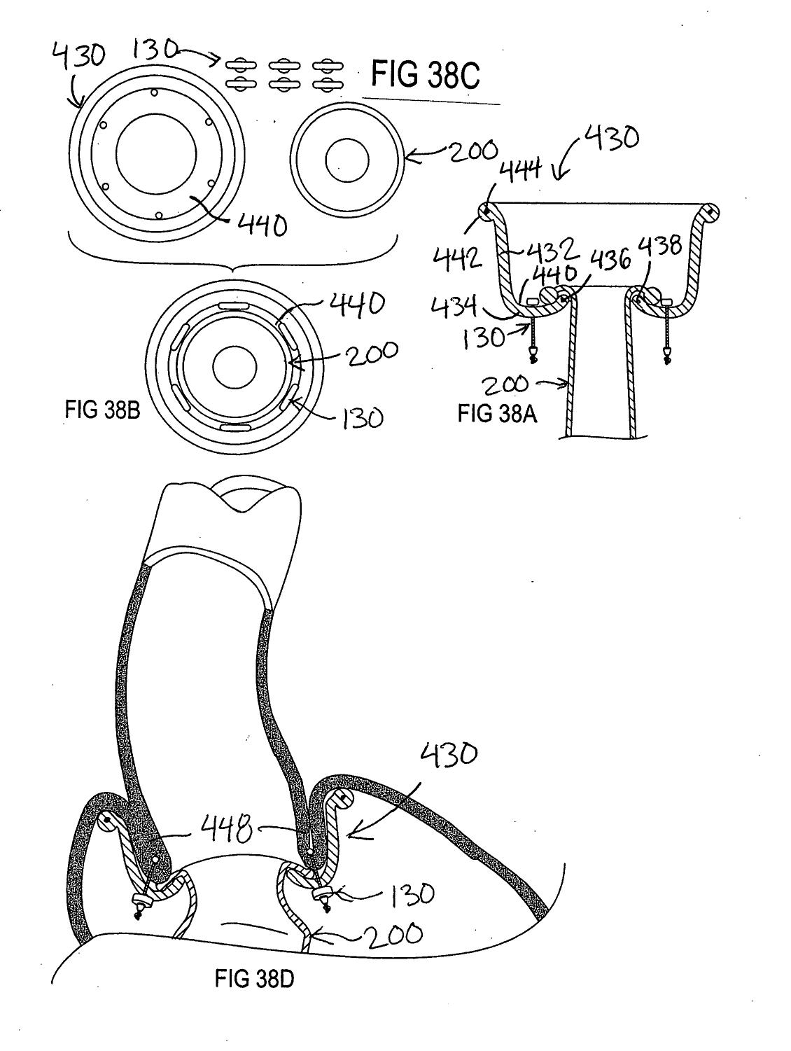

FIGS. 38A-38D show an embodiment of a flexible attachment device 430 for implantable morbid obesity treatment devices. FIG. 38A is a cross section, FIG. 38B is top view and 38C is an exploded top view of the attachment device 430. FIG. 38D shows the attachment device 430 implanted in a patient's stomach near the GEJ. The flexibility or compliance of the attachment device 430 will help to reduce pressure on the tissues in order to avoid complications from tissue necrosis and the like.

The flexible attachment device 430 can be configured as a ring with an L-shaped cross section, with the upper leg of the L forming a cylindrical upper wall 432 and the lower leg of the L forming an annulus or inward-facing lower flange 434. The upper wall 432 is constructed to control, resist and/or recover from collapse due to forces in the stomach wall. In this embodiment the device can be constructed, all or in part, of an ingrowth encouraging material such as Dacron or Teflon (e.g. ePTFE). The material can be woven, knit, felted, expanded or otherwise prepared by means known in the vascular graft and surgical implant art. The material is optionally coated as described herein for encouraging ingrowth and/or resisting the attack of gastric secretions. The inner edge of the flange 434 is shaped to form a sleeve retention ring 436, which is preferably reinforced with a resilient wire 438 of e.g. stainless steel or NiTi. The flange 434 can be constructed of the same materials as the cylinder upper wall 432 or could be constructed from other materials including other biocompatible polymers such as silicone or polyurethane. The outer edge of the flange 434 where it meets the cylindrical upper wall is shaped to form a suture ring 440, also optionally reinforced with a resilient wire. Optionally, the reinforced suture area 440 can be displaced from this meeting point upward along the cylinder wall. In some embodiments, displacement of the suture attachment point can provide space for the sleeve interface. In these embodiments the tissue forming the attachment can provide a resilient sleeve retention, which may be particularly advantageous in preventing upward displacement of the sleeve interface.

Optionally, the upper edge of the cylindrical upper wall 432 is shaped to form a ring 442, optionally reinforced with a resilient wire 444 to help maintain the geometry of the attachment device and to help form a seal against the gastric wall. The inward-facing surfaces 446 of the cylindrical upper wall 432 and the flange 434 are configured for contacting a plication 448 formed in the gastric wall. Optionally, the cylindrical upper wall may have slits or cutouts to increase the flexibility or compliance of the attachment device. Optionally, all or a portion of the tissue contacting surfaces 446 may be made from or covered with a material that encourages tissue ingrowth. Optionally the tissue contacting surfaces 446 may include holes, cavities or opening that encourage tissue ingrowth. These holes, cavities and/or openings may optionally extend completely through the wall of the device.

Preferably, the entire structure of the attachment device 430 is collapsible and expandable so that it can be easily passed through the esophagus in a folded, compressed or collapsed state and re-expanded once it is in the patient's stomach. The attachment device 430 is attached to a plication 448 formed in the gastric wall using endoscopic methods with T-tag fasteners 130, sutures or other fastening means. The sutures or suture tails of the T-tag fasteners 130 may be tied around the suture ring 440, or fastened with snap cap or other suture lock.

In an alternate embodiment, the L-shaped cross section of the attachment device can be reversed so that the lower leg of the L forms an outward-facing lower flange with the sleeve retention ring located at the outer edge of the flange. This alternate geometry of the attachment device will be particularly useful for situations where the implanted device has a larger diameter than the attachment point, as discussed above. Other sleeve interface methods as described herein, the referenced provisionals, and the parent application can also be used as alternatives to an L-shaped flange.

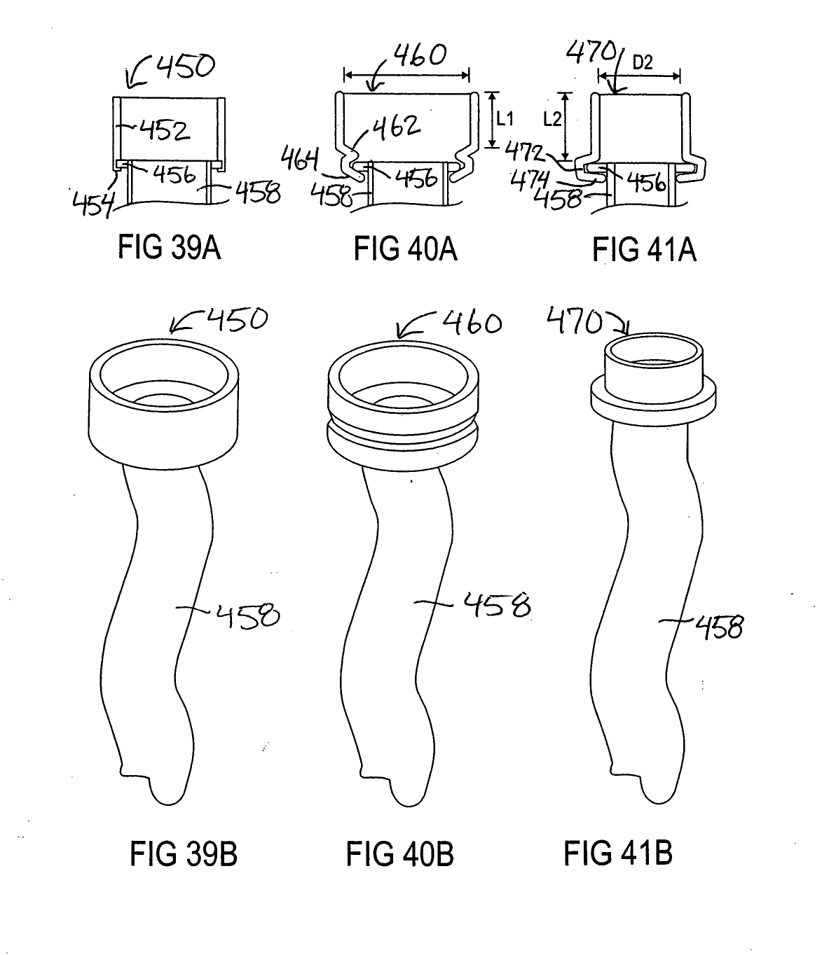

FIGS. 39A-39B show another embodiment of a flexible attachment device 450. The attachment device 450 has a generally cylindrical outer wall 452 with an inward-facing lower flange or ridge 454, and the upstream end of the gastrointestinal sleeve device 458 has a corresponding outward-facing upper flange or ridge 456. This embodiment of the attachment device is preferably formed of a flexible biocompatible polymer such as silicone or polyurethane, which may optionally be reinforced with Dacron or other fabric. The material is optionally coated as described herein for encouraging ingrowth and/or resisting the attack of gastric secretions. All or a portion of the tissue contacting surfaces may be made from or covered with a material that encourages tissue ingrowth. The attachment device 450 is passed through the esophagus and attached to the stomach wall, preferably near the GEJ. The attachment device may include a sewing ring or other features, such as those described herein, to facilitate attachment to a plication or directly to the unplicated stomach wall. The gastrointestinal sleeve device 458 is then passed through the esophagus and into the stomach and intestine. The upper flange 456 on the sleeve device 458 is held by the lower flange 454 of the attachment device 450. Optionally, the upper flange 456 on the sleeve device 458 may have a sliding fit with the cylindrical outer wall 452 to allow for relative motion between the sleeve device 458 and the attachment device 450, with the lower flange 454 on the attachment device 450 serving to limit the downward motion of the sleeve device 458. The sliding fit will help to reduce the tension transferred to the gastric wall from the sleeve device 458 and the weight of its contents.

Alternatively, the attachment device may include a means for capturing the upper flange of the sleeve device to reduce or eliminate relative motion between the sleeve device and the attachment device. FIGS. 40A-40B show an embodiment of the attachment device 460 with an inward-facing lip 462 above the lower flange 464 to capture the upper flange 456 of the sleeve device 458. Preferably, the inward-facing lip 462 has a sloped or tapered upper surface to create a smooth transition between the diameter of the attachment device 460 and the sleeve device 458 and to avoid creating an inner shelf that could catch food before it enters the sleeve device 458. The attachment device 460 may include an attachment ring or other features, such as those described herein, the referenced provisionals, and the parent application, to facilitate attachment to a plication or directly to the unplicated stomach wall.

FIGS. 41A-41B show another embodiment of the attachment device 470 with an annular groove 472 above the lower flange 474 for capturing the upper flange 456 of the sleeve device 458. This geometry allows the inner diameter of the attachment device 470 to be matched to the inner diameter of the sleeve device 458, with no internal steps. The attachment device 470 may include a attachment ring or other features, such as those described herein, the referenced provisionals, and the parent application to facilitate attachment to a plication or directly to the unplicated stomach wall.

The attachment devices of FIGS. 38A-38B, 40A-40B, 41A-41B may include an attached or separate attachment ring at the upper end of the cylindrical outer wall for attachment to a plication formed in the stomach wall. The depth of the attachment ring controls the effective diameter and therefore the resistance provided by the stoma that is created when the stomach wall is plicated inside the ring, as shown in the previously described example of FIG. 38D. A deeper attachment ring holds more tissue in the folds of the plication thereby creating a narrower stoma, whereas a shallower attachment ring holds less tissue in the folds of the plication thereby creating a wider stoma and less resistance to flow.

In other variations of these embodiments, the cylindrical walls can taper inward for attaching an implant device with a smaller diameter than the attachment device or they can taper outward for attaching an implant device with a larger diameter than the attachment device.

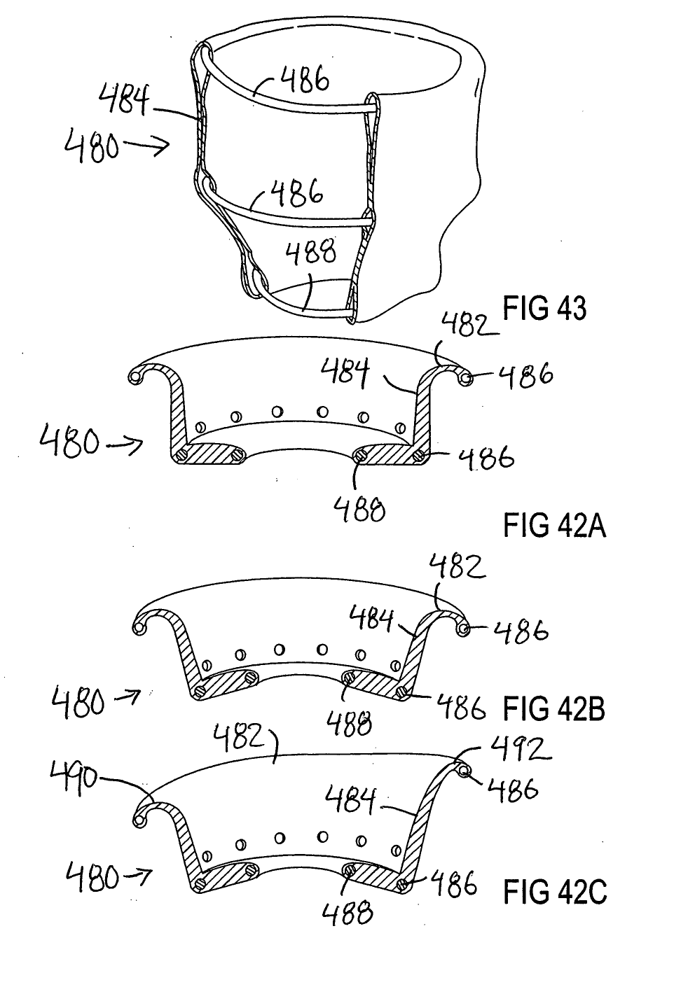

FIGS. 42A-42C show embodiments of an attachment device 480 that are configured with a flaring or outward curve 482 at the upper end of the cylindrical wall 484 to minimize pressure concentrations where it contacts the stomach wall. This flaring can be combined with a thinning of the wall or otherwise increased radial flexibility. The attachment device 480 can be constructed to maintain axial rigidity to control, prevent and/or reverse collapse of the wall in the axial direction.

The cylindrical attachment device 480 of FIGS. 42A-42C may be molded of a flexible biocompatible polymer. Optionally, the cylindrical wall 484 may be supported with a resilient wire-reinforced ring 486 at the upper and/or lower ends. The upper edge 482 of the cylindrical wall 484 flares outward in a 90-180 degree curve to minimize contact pressure on the gastric wall. The flared upper edge 482 of the cylindrical wall 484 may thin toward the edge to increase the flexibility of the wall to further minimize contact pressure on the gastric wall. A third wire-reinforced ring 488 may be provided at the lower end for attachment of a gastrointestinal sleeve device or other implanted device. Alternatively, a lower flange or other features can be molded into the lower end for attaching a gastrointestinal sleeve device in the manner described above.

The flexible cylindrical attachment device 480 of FIG. 39C also illustrates the use of an asymmetric geometry to further minimize contact pressure on the gastric wall. The upper edge 482 of the cylindrical wall 484 flares outward in an approximately 180 degree curve 490 on the side that is implanted toward the lesser curvature of the stomach and an approximately 90 degree curve 492 on the side that is implanted toward the greater curvature of the stomach. Optionally, the cylindrical wall 484 may be supported with a resilient wire-reinforced ring 486 at the upper and/or lower ends and a third wire-reinforced ring 488 or flange may be provided at the lower end for attachment of a gastrointestinal sleeve device. When the attachment device 480 is implanted within a patient's stomach, the flared upper edges 490, 492 conform to the asymmetrical curves of the gastric wall adjacent to the plication to minimize the contact pressure against the wall.

Similar results may be accomplished by using a symmetric geometry and an asymmetric flexibility where a more flexible lesser curve side could maintain a similar pressure with 180 degree deflection as seen on the greater curve side with 90 degree deflection.

The flexible cylindrical attachment device 480 can be constructed from a number of materials and methods. By way of example, FIG. 43 is a cutaway drawing showing the internal construction of a cylindrical attachment device 480 made from woven or knitted material. The cylindrical wall 484 can be woven or knitted in the round or it can be sewn into a cylindrical configuration from one or more pieces of material. The cylindrical wall 484 is preferably supported with a resilient wire-reinforced or elastic filamentous ring 486 at the upper and/or lower ends. The upper edge 482 of the cylindrical wall 484 is optionally rounded or flared outward to minimize contact pressure on the gastric wall as described in the examples above above. A third wire-reinforced ring 488 may be provided at the lower end for attachment of a gastrointestinal sleeve device or other implanted device. Alternatively, a molded upper and/or lower flange or other features can be attached to the lower support ring for attaching a gastrointestinal sleeve device in the manner described above.

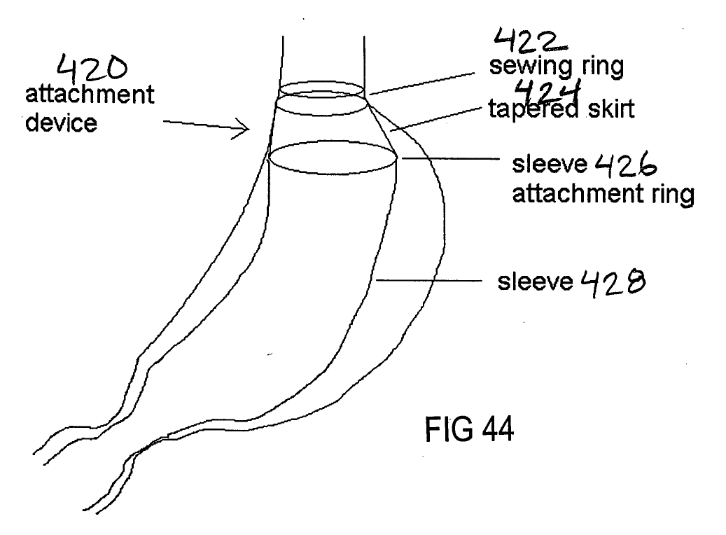

In some clinical circumstances, it may be desirable to implant a treatment device that is larger than the attachment means. FIG. 44 shows an attachment device 420 fastened in the vicinity of the GEJ. At the upstream end of the attachment device 420 is an attachment ring 422 that fastens to the gastric and/or esophageal wall, for example using the apparatus and methods described herein. Suspended below the attachment ring 422 is a gastrointestinal sleeve device 428 for treatment of obesity. The entry of the gastrointestinal sleeve device 428 has a larger diameter than the attachment at the GEJ. To accommodate this, the attachment device 420 has an outward-tapering skirt 424 extending downward from the attachment ring 422. At the bottom edge of the outward-tapering skirt 424 is a device attachment ring 426 or other attachment means for fastening the upstream end of the gastrointestinal sleeve device 428 to the attachment device 420. Preferably, the outward-tapering skirt 424 is made of an impermeable material so that the attachment device provides a fluid-tight seal between the attachment point to the patient and the gastrointestinal sleeve device 428.

Alternatively, the attachment device can provide only a mechanical attachment and the gastrointestinal sleeve device can provide a seal against the gastric wall or a separate sealing device may be provided. In this case, instead of an impermeable skirt, the attachment device may have a suspension frame that provides a mechanical attachment between the attachment ring and the gastrointestinal sleeve device. The suspension frame may be made, for example, from wires or mesh or filaments that provide the necessary mechanical strength, but do not provide a seal. The skirt portion of the attachment device may also be constructed with an impermeable membrane over a suspension frame of this type. Optionally, the suspension frame may include adjustable length tethers for adjusting the distance between the attachment ring and the gastrointestinal sleeve device.

Preferably, the entire structure of the attachment device 420 is collapsible and expandable so that it can be easily passed through the esophagus in a folded, compressed or collapsed state and re-expanded once it is in the patient's stomach. Optionally, the final expanded diameter may be adjustable. Optionally the device 420 may be highly compliant and stretchable where it is attached to the gastric wall.

Although this example shows the implanted device mounted downstream or below the attachment device, in some clinical situations it may be desirable to mount an implant device upstream or above the attachment device. For example, an attachment device in the vicinity of the pylorus may be used to anchor an implant device in the stomach.

### External Sleeve Interface

With (1) a device system where a primary attachment such as a cuff is placed in a tubular duct, e.g. at the GEJ, and a secondary device such as a gastrointestinal sleeve is removably attached to the primary attachment device and (2) placement of the system with a coaxial procedure, e.g. an endoscope passed down the esophagus, placement of the secondary device within the lumen of the primary device can be a simpler approach. However, certain advantages could be obtained if the secondary device were mounted on the exterior of the primary device. Various sleeve geometries with the sleeve portion of the interface being smaller diameter and internally coaxial to the cuff have been previously described.

All of the configurations can be inverted such that the sleeve is of larger diameter and external to the cuff. Similarly, other interface designs such as hooks and eyes disclosed herein can be configured with the sleeve of a larger diameter than the primary mounting cuff. FIG. 45 illustrates an attachment cuff 550 with an external sleeve attachment interface. One of the potential advantages of mounting the sleeve 552 external to the cuff 550 is that such a configuration could be designed to be robust in resisting leaks.

If the proximal portion of the sleeve 552 were less compliant than the distal portion of the cuff 550, internal pressure would press the wall of the cuff 550 into sealing contact with the sleeve 552. In this situation, the seal will be maintained so long as the sleeve 552 stretches less than the cuff 550 as internal pressure increases. Also the distance the cuff 550 and the sleeve 552 overlap can be adjusted to improve interface performance related to leak resistance and/or retention strength. This system can also allow holes or perforations in either the cuff or sleeve in the region of overlap of an unperforated surface without allowing leaks. These holes or perforations may be used to attach the components.

### Separation of Attachment and Sealing

The functions of attachment and sealing can be separated, for example highly compliant attachment can be placed at the GEJ and a sealing connection can be placed upstream in the esophagus. The compliant attachment can be accomplished with gathered or pleated stretchable material. The sealing connection can be configured similar to a covered expandable or self-expanding stent. These separate structures can be improved by structure (e.g. one or more vertical bellows-like pleats) that would allow relative vertical displacement of the sealing and attachment zones.

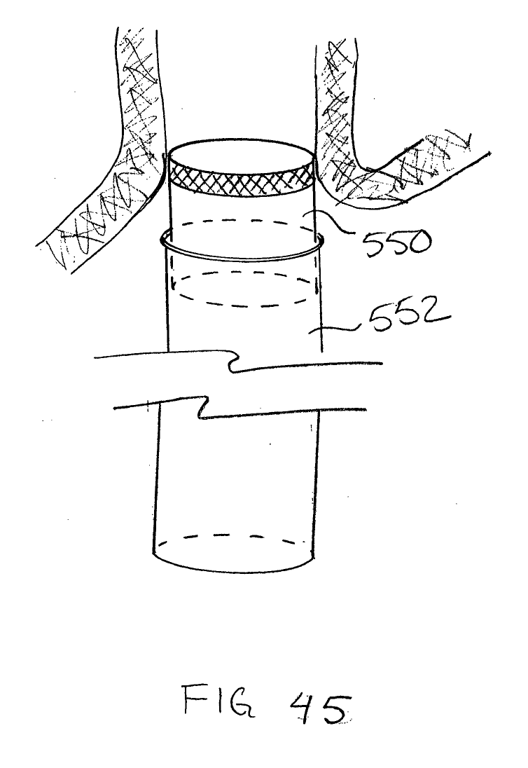

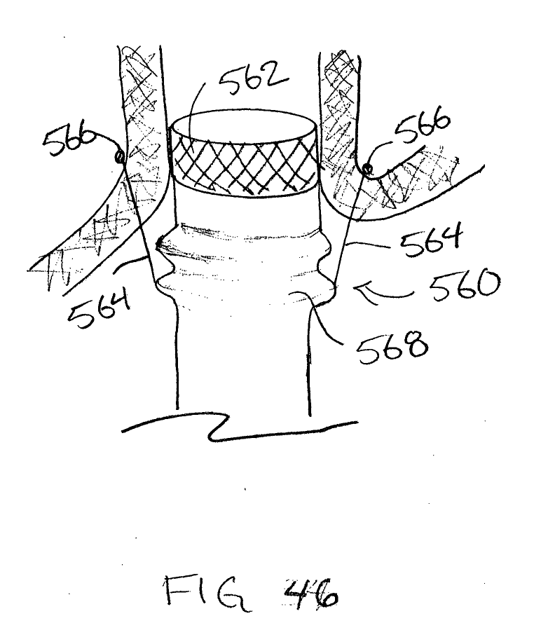

FIG. 46 illustrates an attachment cuff or gastrointestinal sleeve 560 with separation of the attachment and sealing functions. A sealing zone is created, for example using an expandable or self-expanding stent 562 or a compliant material attached with T-tag fasteners. The attachment cuff or gastrointestinal sleeve 560 is attached to the gastric wall downstream of the sealing zone, for example using elongated tethers 564 with transmural T-tags 566. Preferably, the attachment cuff or gastrointestinal sleeve 560 is configured to allow some longitudinal compliance between the sealing zone and the attachment zone. In the example shown, one or more accordion folds 568 create a zone of longitudinal compliance between the sealing zone and the attachment zone.

### Distal Compliance

Similarly, the restrictive component of a morbid obesity treatment system has usually been depicted as being at or distal to the attachment point of the device. Since restrictions are most effective when coupled with a constant or restricted volume proximal to the restriction this suggests that little or no compliance would be preferred at the attachment. Alternatively, the compliance of the attachment can be factored into the definition of the restricted volume. In some clinical situations where high compliance is desirable it could be preferable for the restricted outlet of the restricted volume be placed proximal to a compliant attachment e.g. attachment 562 in FIG. 46. This can allow significant displacement of the compliant attachment without changing (increasing) the restricted volume proximal to the restricted outlet.

Compliant attachment means can be used at or near the GEJ or cardia of the stomach. These attachments can be connected to a restrictive component, which is maintained in a sealing connection with the walls of the GI tract proximal to the attachment. The means used to connect the restrictive component (a device that has a restrictive opening and seals with the walls of the GI tract) do not need to be impervious to masticated food. Sealing means can be passive (for example, an oversized device in a relatively smaller tubular duct) or active (for example, suture, anchor, staple, etc.) However, this sealing is not the primary attachment. It is merely to maintain a seal while the primary compliant attachment resists other forces.

### Compliance Volume Change

Compliance can be problematic if applied to a restrictive GI sleeve system if the volume of the system proximal to a restriction is desired to be limited to a clinically relevant volume. To this end it can be desirable that increases in volume of this compliant attachment area are minimized.

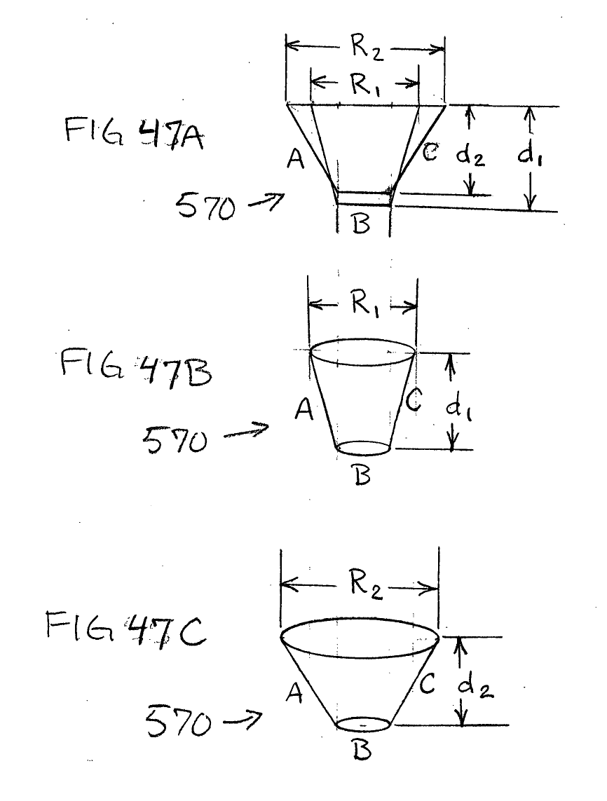

FIGS. 47A-47C illustrate an embodiment of an attachment cuff 570 with controlled compliance volume change. FIG. 47B shows the attachment cuff 570 in an unexpanded state and FIG. 47C shows the attachment cuff 570 in a radially expanded state. FIG. 47A shows cross sections of the attachment cuff 570 in the unexpanded state and the expanded state superimposed to show how the attachment cuff 570 foreshortens as it expands radially to limit the change in volume. As compliant attachment allows R1 to increase to R2, d1 decreases to d2 to limit changes in volume. This can be accomplished with an attachment cuff 570 that is reinforced to allow radial compliance and to resist longitudinal compliance for example by the use of relatively rigid longitudinal ribs. Such ribs can be metal, plastic or filamentous as described herein, the referenced provisionals, and the parent application.

Another structure that can combine compliance with limits in volume increase is a woven mesh. FIGS. 48A-48C illustrate another embodiment of an attachment cuff 572 with controlled compliance volume change. “Shortening” is a known property of self-expanding woven wire mesh stents. A compliant cylinder can be created that shortens in length in response to an expansion of the diameter, as illustrated in FIG. 48B. This can also be used to allow a compliant cone with a highly compliant area for GI attachment and a less compliant area for sleeve attachment, as illustrated in FIG. 48C.

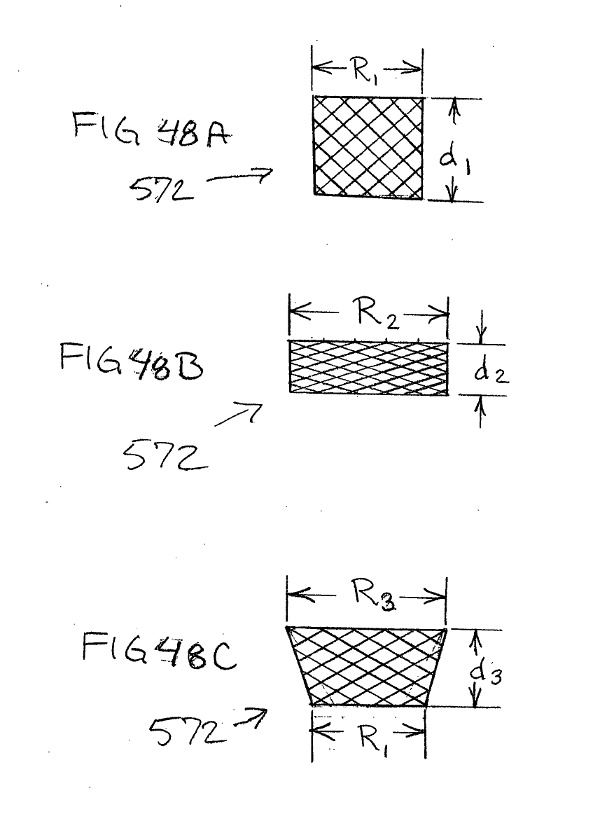

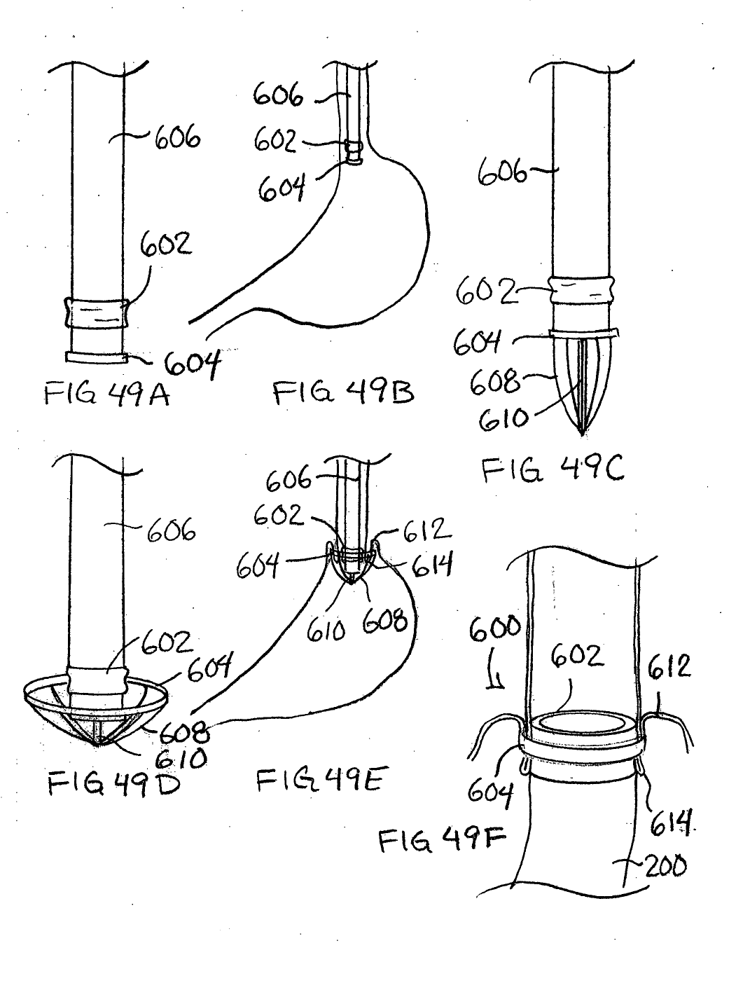

FIGS. 49A-49F illustrate another method and apparatus for attaching a gastrointestinal sleeve device. The apparatus includes a mounting ring 602 that is attachable to the proximal end of a gastrointestinal sleeve device 200 and an elastic band 604 for attaching the ring 602 to a plication 614 of the gastric wall 612 at the GEJ as shown in FIG. 49F. Preferably, the mounting ring 602 is configured to be somewhat flexible, but more rigid than the elastic band 604 and the tissue in the region of the GEJ. The mounting ring 602 is configured to retain the elastic band 604 and prevent detachment. The mounting ring 602 can be made with a slight hourglass shape to it and/or with an annular ridge on the proximal and distal ends to retain the elastic band 604 on the ring 602. The mounting ring 602 can be made with or without a restrictive stoma opening or with any of the adjustable or replaceable stoma devices described in the prior application. The apparatus further includes an endoscopic device 610 for deploying and attaching the mounting ring 602. The mounting ring 602 is positioned around an endoscope or endoscopic delivery tube 606 as shown in FIG. 49A with the endoscopic deployment device 606 carrying the elastic band 604 placed inside a lumen of the endoscope or endoscopic delivery tube 606. The endoscope or endoscopic delivery tube 606 is inserted down the patient's esophagus until the mounting ring 602 is positioned at the GEJ as shown in FIG. 49B. The endoscopic deployment device 610 has struts 608 for expanding and deploying the elastic band 604. The struts 608 may be precurved metal struts (e.g. NiTi or SSt) that expand outwards as they emerge distally from the tube 606 or the struts 608 may be configured to open like the struts of an umbrella. The struts 608 carrying the elastic band 604 emerge from the delivery tube 606 as shown in FIG. 49C, and then expand radially with the elastic band 604 as shown in FIG. 49D. The struts 608 curve proximally to plicate the gastric wall 612 around the outside of the mounting ring 602 as shown in FIG. 49E and release the elastic band 604 around the outside of the plicated tissue 614. The endoscopic deployment device 606 is withdrawn and a gastrointestinal sleeve device 200 is attached to the mounting ring 602 as shown in FIG. 49F using any of the attachment methods described herein or in the prior application.

Another strategy for avoiding excessive pressure on the gastric or esophageal walls and the complications of ischemia and pressure necrosis is to create an extragastric structure for fastening a gastrointestinal sleeve device, mounting ring or other device to. The extragastric structure supports the device and spreads out the force of attachment without any pressure pinching on the tissue to cause ischemia. FIGS. 50A-50B illustrate a method and apparatus using an extragastric structure 650 for attaching a gastrointestinal sleeve device 200. FIG. 50A is a perspective view of an extragastric structure 650 in the form of a support ring made up of multiple segments 652 implanted near the gastroesophageal junction. FIG. 50B is a top view of the extragastric support ring 650 with the wall of the esophagus shown in cross section. In the example shown, the extragastric support ring 650 is configured with 8 segments, but more or fewer segments can be used. Optionally, the diameter of the extragastric support ring 650 can be adjusted to the external diameter of the organ by adding or subtracting segments. In one method, each of the segments is inserted through one or more needle punctures from the inside of the esophagus. Suture tails 664 attached to the segments may be used to attach a gastrointestinal sleeve device 200, mounting ring or other device. Optionally spacers 662 like those described above may be used to further guard against excessive pressure on the gastric or esophageal walls.

The segments 652 may be free floating, which has the advantage of allowing for expansion and contraction of the stomach and esophagus. Alternatively, a means may be provided for linking the segments 652 end-to-end to form a more solid ring, which has the advantage of distributing the forces on the tissues more effectively. One way to do this is by making the segments 652 with interlocking ends so that the segments can be joined together into a ring after they are inserted through the wall in the region of the GEJ.

In an alternate method, the segments can be inserted laparoscopically and assembled into a ring around the exterior of the GEJ. Once the extragastric structure is in place, sutures or other fasteners can be put through the gastric or esophageal wall to capture the ring and attach a gastrointestinal sleeve device, mounting ring or other device to it.

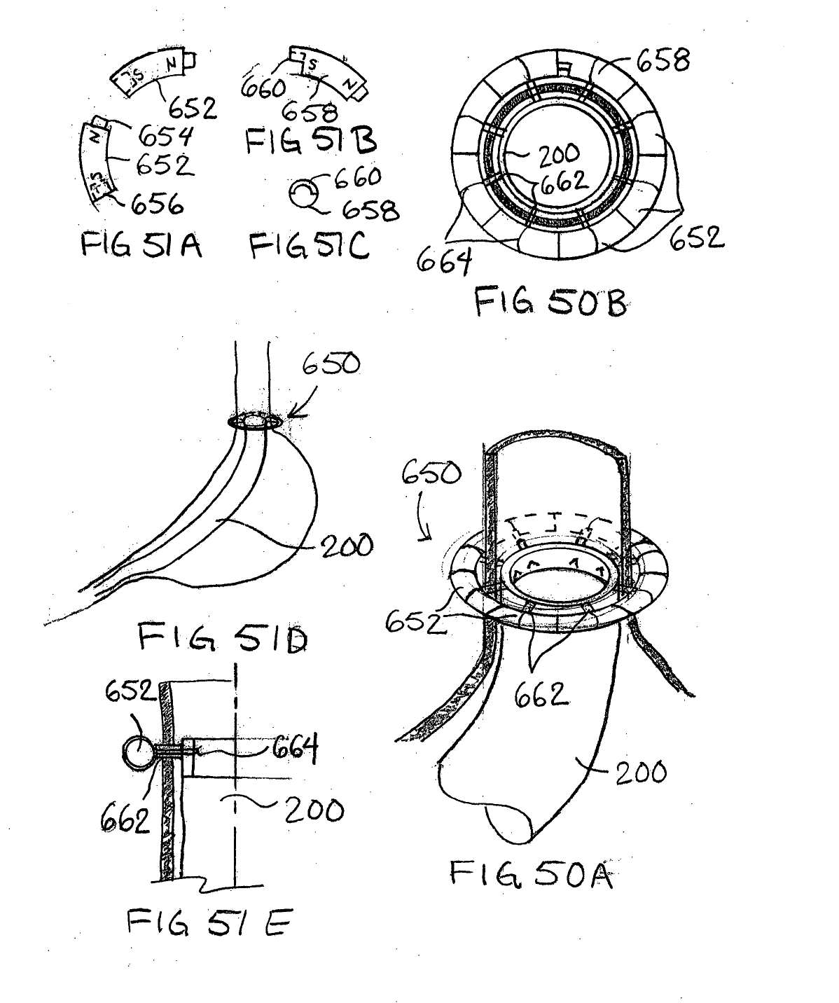

FIGS. 51A-51E show details of one embodiment of an extragastric structure 650 for attaching a gastrointestinal sleeve device 200. In this example, the segments 652 of the ring are made with permanent magnets, such as rare earth magnets, that are arranged with North and South poles on opposite ends of the segments 652. The magnetic poles will automatically align the interlocking ends of the segments after they are inserted through the wall in the region of the GEJ. In the example shown, the segments are made with a male cylindrical member 654 on one end and a female cylindrical member or socket 656 on the other end. When properly aligned, the male 654 and female members 656 will interlock in a ring configuration. The male and/or female members may include detents, barbs or other structures to secure the ring together. FIGS. 51B and 51C show an optional configuration for the final segment 658 of the ring. It is optionally configured so that it only partially interlocks with the adjacent segments so that it is easier to insert. In the example shown, the female cylindrical member or socket 660 only covers approximately 180 degrees of the adjacent male cylindrical member to simplify alignment and insertion of the final segment.

In an example of a method using an extragastric structure, a 4 cm diameter extragastric support ring 650 can be joined to a 3 cm diameter inner attachment ring 200 with 0.5 cm spacers 662 between the inner and outer rings. The gastric and/or esophageal wall, which is typically about 3-5 mm thick at the GEJ, will be captured between the inner 200 and outer 650 rings with very little pressure at the attachment. Furthermore, any force or tension transferred to the rings from a gastrointestinal sleeve or other device attached to them will be distributed around the periphery of the organ by the inner and outer rings, reducing the tension and attachment pressure on the tissue.

Other methods may be used for forming and/or placing extragastric structures via a peroral route. In one example, a guidewire with a preset curve is inserted through a needle puncture in the wall in the region of the GEJ. The guidewire can be configured as a small diameter wire, constructed for example of SSt or NiTi, with a ball or knob on the distal end to avoid inadvertently piercing the tissues with the wire. Optionally, the guidewire may be coated with a low friction coating. The guidewire is curved so that it will curl around the outside of the GEJ to form a 360 degree loop. Optionally, a wire snare or other tool inserted through the same needle puncture or an adjacent needle puncture may be used to capture the distal end of the guidewire as it comes around full circle. A tubular extragastric member is then inserted over the guidewire so that it makes a complete circle around the GEJ. Optionally, the leading and trailing ends of the extragastric member may be joined together to form a continuous loop. Sutures, T-tags or other fasteners can be inserted through the wall at selected points around the inside of the GEJ to attach to the extragastric member. Once the extragastric member is securely in place, the guidewire can be withdrawn. Alternatively, the extragastric member itself may be made with a present curve so that it will curl around the outside of the GEJ to form a 360 degree loop without the aid of a guidewire.

In an alternative method, suture loops or other fasteners with loops on them could be inserted through the wall of the GEJ prior to insertion of the guidewire and/or extragastric member. The guidewire and/or extragastric member will be passed through the loops on the exterior surface of the GEJ; then the loops can be tightened to fasten the extragastric member in place. The sutures or fasteners could be used to directly or indirectly fasten another device to the extragastric structure as described above.

In a variation on this method, inflatable balloons surrounded by suture loops or the like can be inserted through the wall at the GEJ and inflated on the extragastric surface. The balloons would serve to move any adjacent tissues out of the way and to facilitate passage of the guidewire through the suture loops. In one embodiment, the wire would puncture through the balloons as it passes through the loops. In another embodiment, the balloons could be deflated, leaving the loops open for the wire to pass through. In yet another embodiment, the balloons could be configured so that they would hold the loops open for the wire to pass through without occluding the loops. For example, small toroidal balloons or U-shaped balloons could be used to hold the loops open. Likewise, a toroidal or U-shaped mechanical support can be used in place of the balloons.

In another variation on this method, a wire or filament with a magnet or magnetic material at the tip can be inserted through a needle puncture at the GEJ. A magnetic or electromagnetic manipulator inside the esophagus can be used to guide the magnetic tip around the exterior of the GEJ to form a loop. The wire or filament may have a preset curve as in the previous examples to facilitate this step. Once the extragastic structure is in place, the magnetic tip of the wire can be withdrawn in small increments and used to guide the placement of sutures or other fasteners into or around the extragastric structure using a magnetic detector.

### Tissue Prestrengthening

In some clinical situations the gastroesophageal junction, or GEJ, is a preferred attachment point for a gastroesophageal sleeve device or attachment device. Attachment at the GEJ excludes all gastric secretions from the interior of the gastrointestinal sleeve device to separate ingested food and liquids in the sleeve device from all digestive secretions. The gastroesophageal junction is one of the preferred attachment sites because the tissue wall is relatively thick at this location and it is relatively easy to access via a per oral route. More specifically, the non-glandular tissue at the squamo-columnar junction (a zone of tissue that is considered to be at the transition of the esophagus to the stomach that is near the GEJ) is the strongest tissue in this region and is currently thought to be the best place to attach a device, for example using T-tags, sutures or other fasteners.

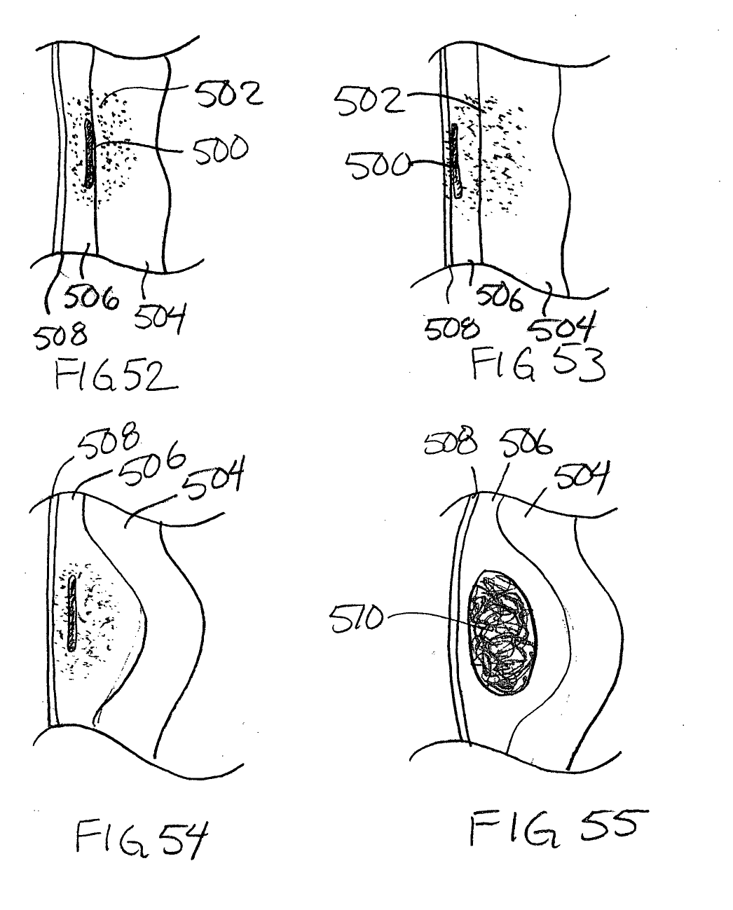

In some clinical situations it may be beneficial to prestrengthen the tissue prior to implantation of a device such as a gastrointestinal sleeve device. For example, energy can be delivered to by RF, ultrasound or other known method to induce an inflammatory, coagulative or necrotic tissue strengthening reaction. Alternatively, placement of material in the muscularis of the stomach wall could generate a foreign body reaction that would progress from inflammation, to granulation of tissue and then to fibrosis. The tissue may initially weaken due to the inflammatory response, but the resulting fibrotic growth will strengthen the tissue. This effect could be enhanced by the choice of material an/or coatings, e.g. an acidic material or coating. The materials could be delivered endoscopically with a needle device through the biopsy channel of an endoscope. The needle delivery device could optionally also deliver an ink, dye or other marking means to facilitate location of the prestrengthened areas. Tissue reaction could take place in days, with 7-14 days being an approximate delay between prestrengthening and attachment procedures. FIGS. 52-53 show examples of tissue prestrengthening in the gastrointestinal system. In FIG. 52, the material 500 has been delivered into the gastric wall just under the mucosa 504. The granulation and fibrosis 502 start at this point and progress through the muscularis 506 to strengthen the tissue and prepare it for attachment of an implantable device. In FIG. 53, the material 500 has been delivered deeper into the gastric wall close to the serosa 508. The granulation and fibrosis 502 start at this point and progress through the muscularis 506 to strengthen the tissue and prepare it for attachment of an implantable device. Optionally, tissue prestrengthening can be accomplished by inserting a fibrosis inducing agent with a suture tail attached to it to allow it to be retrieved or used to guide a subsequent attachment to the prestrengthened location after it has had the desired affect on the tissue wall.

Material injectable to prestrengthen tissue could be:
1) liquid where natural processes would remove/break down or otherwise dispose of the liquid when it has completed its function;
2) biodegradable or dissolvable where natural processes would remove the material when it has completed its function; or
3) permanent where the material might be incorporated into the tissue to provide increased strength.

All of the prestrengthening strategies described could be used also be applied at the time of the attachment procedure to enhance strength of the attachment.

The methods and apparatus described for tissue strengthening would be expected to result in some degree of tissue thickening as new collagen and fibrotic material will be deposited and/or generated at the location of the foreign body reaction. This tissue thickening would be expected to continue as long as viable tissue receives additional stimulus. This can be controlled by use of timed release chemical stimulants and stimulants with known and potentially controllable half lives. Tissue thickening and tissue strength may be related and may facilitate durable attachment, however tissue thickening may be an inherently desirable result in some clinical situations. FIG. 54 shows an example of tissue thickening in the gastrointestinal system as a result of a material 500 injected or delivered into the gastric wall. Optionally, a suture or other filament 512 may be connected to the material to allow it to be retrieved or used to guide a subsequent attachment to the prestrengthened location after it has had the desired affect on the tissue wall and to halt additional tissue thickening.

Currently, tissue bulking agents are injected at or near the GEJ to treat GERD. Injection of non-bulking materials that initiate tissue thickening could accomplish the same end result. If the thickened tissue was, by itself or in conjunction with a supporting structure, to form a restrictive stoma, there could be specific advantages relative to a mechanical stoma. FIG. 55 shows an example of tissue thickening in the gastrointestinal system using a bulking material 510 injected or delivered into the gastric wall.

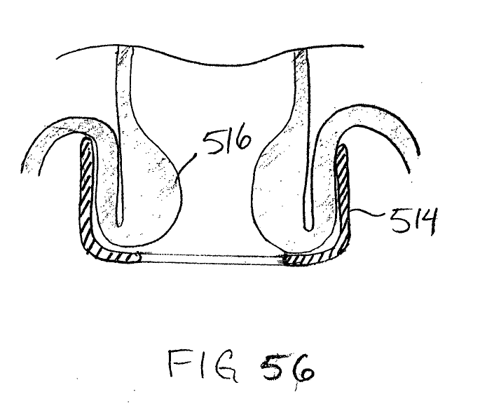

In the case of a flexible ring mounting cuff, for example those shown in FIGS. 38-42, the cuff secures gastric tissue within the lumen of the ring. The tissue within the ring could be thickened to create a restrictive stoma. The restrictive stoma could be adjusted using known techniques, such as balloon dilation. Time control and/or reversible tissue thickening could also control the stoma opening. FIG. 56 shows an example of tissue thickening 516 used to create a restrictive stoma within a mounting cuff 514 attached at the GEJ.

Tissue prestrengthening and/or thickening can be accomplished by inserting a fibrosis inducing agent with a suture tail attached to it. The fibrosis inducing agent will preferably also act as a scaffold for strength. The tail will allow easy identification of the location that has been strengthened for retrieval or guidance of follow on attachment procedures. Once tissue has strengthened, cuff and sleeve can be placed in a single combined procedure as the prestrengthened tissue will not require additional healing time to hold.

Other approaches to induce tissue prestrengthening and/or thickening include:

Circumferential ablation (RF, microwave, ultrasound, etc)

Over-dilation

Circumferential abrasion

Circumferential exposure to agent

An advantage of a circumferential area of tissue strengthening is that it only needs to be located along a vertical axis for subsequent attachment procedures.

Alternately or in addition to the above pre-strengthening of tissue, tissue can be treated to reduce its ability to move or stretch. This can be advantageous in that tissue that has limited stretch or motion may have less impediments to attachment. Tissue that has limited stretch or motion may impose fewer forces on an attached device and therefore impose less pressure that may lead to attachment failure. Furthermore, tissue that has limited stretch or motion may allow attachment of less compliant devices which can provide for advantages foe example simplified sealing.

Means described above to strengthen tissue can also help to limited GI tissue stretch and motion. Other methods that could be applied to reducing stretch and motion, and also for pre-strengthening, include the application of energy for example, by RF, ultrasound or laser. Means that include time release elements as well known in the art of drug eluting vascular stents and birth control devices can be used to provide and/or maintain a long lasting effect (reducing motion and stretch). Such time release means can optionally be combined with fasteners, permanent or replaceable attachment cuffs or proximal sleeve interfaces. Such time release means can optionally be combined with permanently implanted pre-strengthening materials where the material might be incorporated into the tissue to provide increased strength.

### Suture Lengthening

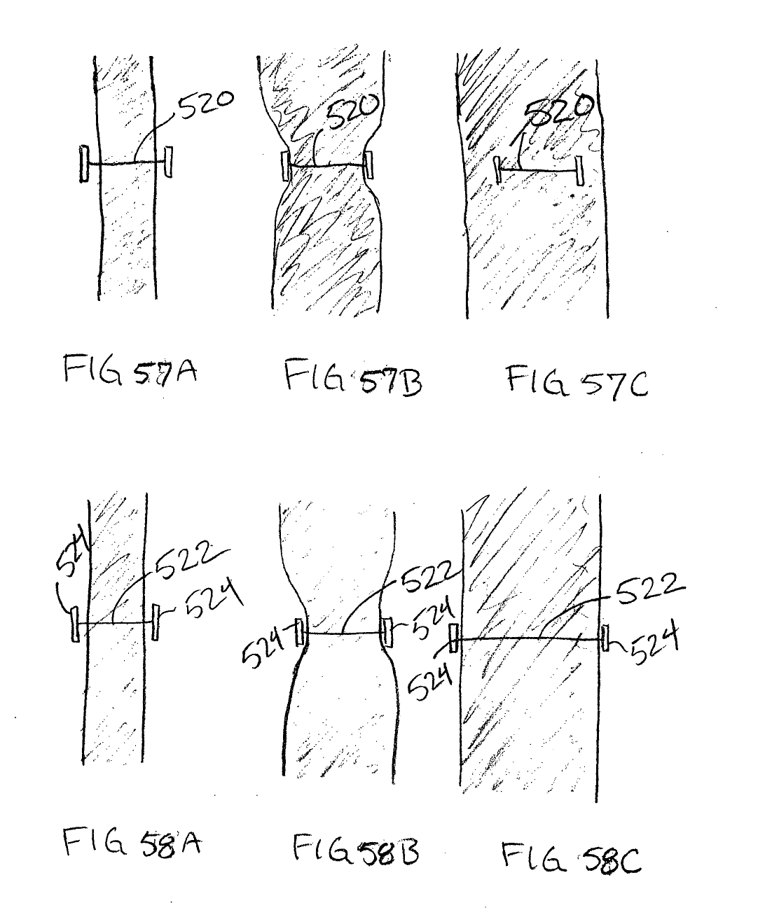

In some clinical situations when using a transmural attachment, the wall of tissue may thicken after placement of the attaching device. FIGS. 57A-57C show the effect of tissue thickening on a fixed length suture or fastener 520. In some cases this can progress to encapsulation. This thickening can result in increased tissue strength due to collagen deposition and/or fibrosis.

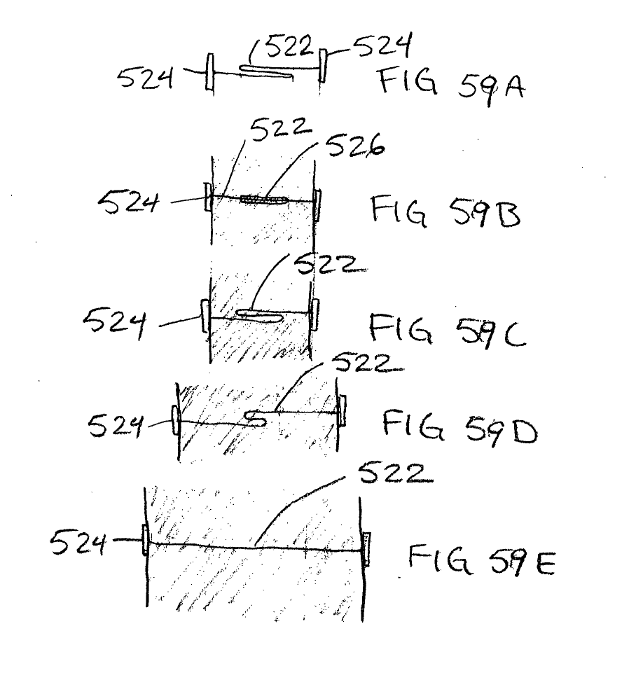

In some clinical situations it can be advantageous to maintain the attachment on the surface of the tissue to take advantage of the added strength of the thickened wall. FIGS. 58A-58C show controlled suture lengthening to compensate for tissue thickening. One manner in which this could be accomplished would be by using a suture 522 connecting the attachments 524 on either side of the tissue wall that would stretch as the tissue thickens.

One configuration of material that could have advantageous performance would:
1) not stretch for the initial period, for example 24-48 hours;
2) stretch at a relatively low force for the next period, for example 7-14 days; then
3) not stretch after the second period.

This performance would be based upon a clinical situation where tissue proliferation (wall thickening) occurs between days 2 and 14.

Alternatively, the material could:
1) not stretch for the initial period, for example 24-48 hours;
2) allow lengthening to 2× length at any time after the initial period, for example 48 hours; then
3) not stretch beyond 2× length.

FIGS. 59A-59E illustrate an embodiment of a fastener with controlled suture lengthening to compensate for tissue thickening. In this example, the fastener is configured to allow approximately 2× lengthening over a controlled time period and then to resist further lengthening. This can be accomplished as follows:

A portion of the suture 522 is folded 3× as shown in FIG. 59A. A dissolvable or resorbable coating or adhesive 526 is added to the overlapping portion of the suture 522 to initially resist lengthening after implantation as shown in FIG. 59B. The coating 526 will dissolve or resorb after a predetermined period of time, releasing the folded portion of the suture 522 as shown in FIG. 59C. After dissolution of the coating 526, the suture 522 will lengthen without significant resistance as shown in FIG. 59D. FIG. 59E shows the suture 522 fully extended to its final length.

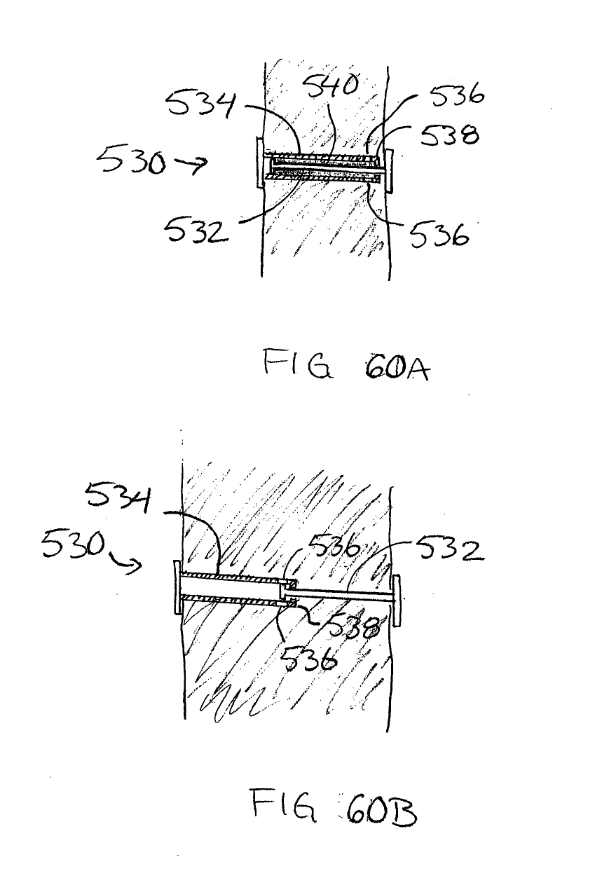

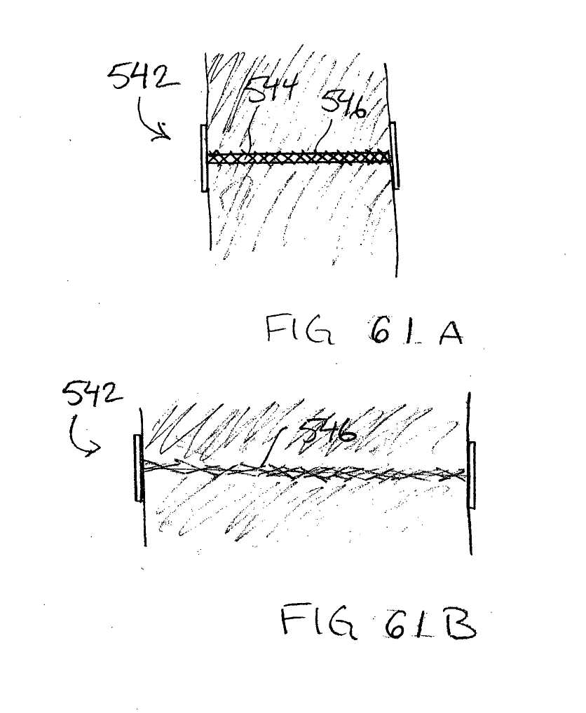

Lengthening could also be accomplished with a coaxial system. FIGS. 60-60B illustrate another embodiment of a fastener 530 with a coaxial system for controlled suture lengthening to compensate for tissue thickening. The fastener 530 has a linear inner member 532 and a tubular outer member 534 in a telescoping coaxial arrangement. Ports 536 in the tubular outer 534 member allow fluid entry. A stop or detent 538 at the ends of the inner member 532 and outer member 534 prevent separation of the fastener 500. Similar to the system previously described, a dissolvable or degradable material 540 can be used to control lengthening. By restricting or controlling exposure of the biodegradable material to body fluids, the rate of lengthening can be optionally controlled.

The principle of shortening/lengthening of a braided mesh can be applied to an automatically lengthening suture. FIGS. 61A-61B illustrate another embodiment of a fastener 542 using this principle for controlled suture lengthening to compensate for tissue thickening. A dissolvable inner core 544 holds the braided mesh suture 546 open, keeping the fastener 542 in a shortened configuration as shown in FIG. 61A. After the inner core 544 dissolves, the braided mesh suture 546 can lengthen as shown in FIG. 61B. Lengthening is accomplished by a change in angle of the braided fibers as the inner core dissolves, allowing the diameter to shrink.

An optimized yielding suture system for GI attachment could have differing responses to short impulse and long continuous loads. Viscoelastic polymer systems such as Tempurfoam resist short term impulse loads and yield to slow steady long term loads. Resistance to short term loads and yielding to long term loads could be advantageous in clinical situations where there can be temporary short term loading due to overeating while yielding to long term loads could relieve overpressurization of tissue to enable tissue perfusion to avoid tissue necrosis due to pressure and/or ischemia.

In other clinical situations yielding to short term transient loads may facilitate secure attachment when coughing, retching, swallowing etc. occur. Resistance and/or recovery—in the face of lower and slower forces can return the suture to a normal length, facilitating leak free securing of an attachment.

Yield points and/or recovery points should be selected so attachment forces do not exceed the acute pull out force of a suture and/or T-tag. Acute pull out forces can be in the range of 3-9 pounds depending on the suture/T-tag configuration. This is of particular concern for short term impulse loads. Loads applied over longer periods should be selected to avoid ischemia and/or pressure necrosis. These forces can be very low and can be less than 1 pound.

In an ideal situation, a suture system will recover to a set length which results in no force applied to the tissue over extended periods of time. Attachment can resist forces between necrosis force/pressure if they are transient and allow tissue time for healing and/or recovery. A system whereby recovery after yielding occurs in a non-continuous manner thereby allowing tissue healing and/or recovery could be desirable in many clinical situations. This may be accomplished by materials that respond to outside stimulus (e.g. electrical, chemical, magnetic, etc.) that can be applied intermittently.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. A gastrointestinal implant, comprising:
an elongate, tubular body, having a proximal end, a distal end, a length adapted to extend distally from gastroesophageal junction, and
a diameter less than the diameter of the gastrointestinal lumen;
an attachment ring about the proximal end of the elongate, tubular body; and
a plurality of tissue fasteners extending from the attachment ring, wherein each tissue fastener comprises:
an elongate member adapted to enter a tissue wall adjacent to the attachment ring; and
a transverse retention element joined to the elongate member for resisting movement of the elongate member.

2. The gastrointestinal implant of claim 1, wherein the transverse retention element has a circular configuration.

3. The gastrointestinal implant of claim 1, wherein the transverse retention element has a linear configuration.

4. A gastrointestinal implant, comprising:
an elongate, tubular body, having a proximal end, a distal end, a length of at least about 60 cm and a diameter less than about 4 cm;
an attachment ring about the proximal end of the elongate, tubular body; and
a plurality of tissue fasteners extending from the attachment ring, wherein each tissue fastener comprises:
an elongate member adapted to interface with a tissue wall adjacent to the attachment ring; and
a transverse retention element joined to the elongate member for resisting movement of the elongate member.

5. The gastrointestinal implant of claim 4, wherein the transverse retention element has a circular configuration.

6. The gastrointestinal implant of claim 4, wherein the transverse retention element has a linear configuration.
